(19) United States
(12) Patent Application Publication
(10) Pub. No.: US 2015/0045410 A1
(43) Pub. Date: Feb. 12, 2015

(54) METHOD OF REGULATING CFTR EXPRESSION AND PROCESSING

(71) Applicant: University of Iowa Research Foundation, Iowa City, IA (US)

(72) Inventors: Paul McCray, Iowa City, IA (US); Shyam Ramachandran, Iowa City, IA (US); Yi Xing, Iowa City, IA (US); Michael Welsh, Iowa City, IA (US); Mark Behlke, Coralville, IA (US)

(21) Appl. No.: 14/376,810

(22) PCT Filed: Feb. 6, 2013
(86) PCT No.: PCT/US13/24985

§ 371 Date: Aug. 5, 2014

Related U.S. Application Data
(60) Provisional application No. 61/595,493, filed on Feb. 6, 2012.

(51) Int. Cl. C12N 15/113; A61K 31/7088; A61K 45/06

(52) U.S. Cl. CPC C12N 15/113; A61K 45/06; A61K 31/7088; C12N 2310/141; C12N 2310/11; C12N 2310/14; C12N 2320/31
USPC 514/44 A; 435/375

(57) ABSTRACT

The present invention relates to therapeutic agents comprising miR-138, a miR-138 mimic, a SIN3A RNAi molecule, or a an anti-SIN3A RNAi molecule, and/or an anti-SIN3A antisense oligonucleotide (ASO) or other agent that suppresses SIN3A expression, a small molecule drug that interferes with SIN3A activity or whose actions mimic the biological effects of SIN3A suppression and methods of use of these therapeutic agents to treat cystic fibrosis.

### RELATED APPLICATION

This application claims priority under 35 U.S.C. 119(e) to provisional application U.S. Ser. No. 61/595,493 filed Feb. 6, 2012, which application is incorporated hereby by reference.

### STATEMENT REGARDING FEDERALLY SPONSORED RESEARCH

This invention was made with government support under grant R21 HL104337 awarded by the National Institutes of Health. The government has certain rights in the invention.

### SEQUENCE LISTING

The instant application contains a Sequence Listing which has been submitted in ASCII format via EFS-Web and is hereby incorporated by reference in its entirety. Said ASCII copy, created on Jan. 30, 2013, is named 17254W01.txt and is 27,267 bytes in size.

### BACKGROUND OF THE INVENTION

Cystic fibrosis (also known as CF or mucoviscidosis) is a common recessive genetic disease which affects the entire body, causing progressive disability and often early death. The name cystic fibrosis refers to the characteristic scarring (fibrosis) and cyst formation within the pancreas, first recognized in the 1930s. Difficulty breathing is the most serious symptom and results from frequent lung infections that are treated with, though not cured by, antibiotics and other medications. A multitude of other symptoms, including sinus infections, poor growth, diarrhea, and infertility result from the effects of CF on other parts of the body.

CF is caused by a mutation in the gene that encodes the cystic fibrosis transmembrane conductance regulator (CFTR) protein. This gene is required to regulate the components of sweat, digestive juices, and mucus. The CFTR protein, when positioned properly in the cell membrane, opens channels in the cell membrane. When the channels open, anions, including chloride and bicarbonate are released from the cells. Water follows by means of osmosis. Although most people without CF have two functional copies (alleles) of the CFTR gene, only one is needed to prevent cystic fibrosis (i.e., CF is an autosomal recessive disease). CF develops when neither allele can produce a functional CFTR protein. The most common mutation, ΔF508, is a deletion (Δ) of three nucleotides that results in a loss of the amino acid phenylalanine (F) at the 508th (508) position on the protein. The ΔF508 mutation can prevent the CFTR from moving into its proper position in the cell membrane. This mutation causes an abnormal biogenesis and premature degradation of CFTR protein by the cells quality control system and, as a result, there is a paucity/absence of CFTR in the apical membrane of CF epithelial cells. This results in a decreased anion permeability across CF epithelia.

CF is most common among Caucasians; one in 25 people of European descent carry one allele for CF. Approximately 30,000 Americans have CF, making it one of the most common life-shortening inherited diseases in the United States. Individuals with cystic fibrosis can be diagnosed before birth by genetic testing, or by a sweat test in early childhood. Ultimately, lung transplantation is often necessary as CF worsens. The ΔF508 mutation accounts for two-thirds (66-70%) of CF cases worldwide and 90 percent of cases in the United States; however, there are over 1,500 other mutations that can produce CF.

Currently, there are no cures for cystic fibrosis, although there are several treatment methods. The management of cystic fibrosis has improved significantly over the years. While infants born with cystic fibrosis 70 years ago would have been unlikely to live beyond their first year, infants today are likely to live well into adulthood. The cornerstones of management are proactive treatment of airway infection and inflammation, and encouragement of good nutrition and an active lifestyle. Management of cystic fibrosis is aimed at maximizing organ function, and therefore quality of life. At best, current treatments delay the decline in organ function. Targets for therapy are the lungs, gastrointestinal tract (including pancreatic enzyme supplements), the reproductive organs (including assisted reproductive technology (ART)) and psychological support.

The most consistent aspect of therapy in cystic fibrosis is limiting and treating the lung damage caused by thick mucus and infection, with the goal of maintaining quality of life. Intravenous, inhaled, and oral antibiotics are used to treat chronic and acute infections. Mechanical devices and inhalation medications are used to alter and clear the thickened mucus. These therapies, while effective, can be extremely time-consuming for the patient. One of the most important battles that CF patients face is finding the time to comply with prescribed treatments while balancing a normal life.

In addition, therapies such as transplantation and gene therapy aim to cure some of the effects of cystic fibrosis. Gene therapy aims to introduce normal CFTR to airway epithelial cells. There are two types of CFTR gene therapies under development, the first uses viral vectors (adenovirus, adeno-associated virus or retrovirus) and the second uses plasmid DNA in formulations such as liposomes. However there are problems associated with both of these methods involving efficiency (liposomes insufficient plasmid DNA) and delivery (virus vectors provoke an immune responses).

Accordingly, a more effective, simple-to-administer, and efficient treatment for CF is needed.

### SUMMARY OF THE INVENTION

In certain embodiments, the present invention provides a method of increasing the amount of functional CFTR on the cell membrane by reducing the level of SIN3A in a CF cell. In one embodiment the method comprises contacting the cell with a therapeutic agent, wherein the agent comprises miR-138, a miR-138 mimic. In another embodiment, the method comprises contacting the cell with a therapeutic agent, wherein the agent comprises an anti-SIN3A RNAi molecule, an anti-SIN3A antisense oligonucleotide (ASO), or other agent that suppresses SIN3A expression, which methods are well-known to those with skill in the art. In yet another embodiment, the method comprises contacting the cell with a therapeutic agent, wherein the agent comprises a small molecule drug that interferes with SIN3A activity or whose actions mimics the biological effects of SIN3A suppression. In certain embodiments, SIN3A expression is inhibited by at least about 10%, 20%, 30%, 40%, 50%, 60%, 70%, 80%, 90% 95%, or 99%. In certain embodiments, small molecule drugs that inhibit SIN3A activity are used to inhibit SIN3A, such as by inhibiting translation of SIN3A or by directly interfering with function of the SIN3A protein. In yet another embodiment the therapeutic agent does not alter SIN3A levels or activity but instead affects activity of a downstream SIN3A target gene or protein that is involved in CFTR processing.

In certain embodiments, the present invention provides a method of increasing ΔF508 CFTR expression in a cell comprising contacting the cell with a therapeutic agent, wherein the agent comprises miR-138, a miR-138 mimic, an anti-SIN3A RNAi molecule, and/or an anti-SIN3A antisense oligonucleotide (ASO) or other agent that suppresses SIN3A expression, a small molecule drug that interferes with SIN3A activity or whose actions mimic the biological effects of SIN3A suppression. As used herein an “RNAi molecule” is an RNA molecule that functions in RNA interference (e.g., siRNA, shRNA or DsiRNA).

In certain embodiments, the present invention provides a method of generating a CFTR anion channel in a cell comprising contacting the cell with a therapeutic agent, wherein the agent comprises miR-138, a miR-138 mimic, an anti-SIN3A RNAi molecule, and/or an anti-SIN3A antisense oligonucleotide (ASO) or other agent that suppresses SIN3A expression, a small molecule drug that interferes with SIN3A activity or whose actions mimic the biological effects of SIN3A suppression.

In certain embodiments, the present invention provides a method for enhancing anion transport in epithelial cells, comprising contacting epithelial cells with a therapeutic agent to alleviate the symptoms of CF, wherein the agent comprises miR-138, a miR-138 mimic, an anti-SIN3A RNAi molecule, and/or an anti-SIN3A antisense oligonucleotide (ASO) or other agent that suppresses SIN3A expression, a small molecule drug that interferes with SIN3A activity or whose actions mimic the biological effects of SIN3A suppression. In certain embodiments, the anion is chloride.

In certain embodiments the present invention provides a method of enhancing CFTR protein processing in a cell comprising contacting the cell with a therapeutic agent, wherein the agent comprises miR-138, a miR-138 mimic, an anti-SIN3A RNAi molecule, and/or an anti-SIN3A antisense oligonucleotide (ASO) or other agent that suppresses SIN3A expression, a small molecule drug that interferes with SIN3A activity or whose actions mimic the biological effects of SIN3A suppression. This refers to all steps after initial protein translation from mRNA that allow for the production of a mature membrane channel. This includes the core and terminal glycosylation steps in the endoplasmic reticulum, with subsequent passage through the Golgi apparatus, and vesicular trafficking to the cell membrane. Terminal glycosylation of CFTR (termed “band C”) is evidence of successful processing. In certain embodiments, the cell is a CF epithelial cell, such as an airway epithelial cell (e.g., a lung cell, a nasal cell, a tracheal cell, a bronchial cell, a bronchiolar or alveolar epithelial cell). In certain embodiments, the airway epithelial cells are present in a mammal. In certain embodiments, the cell produces a CFTR protein having a deletion at position 508.

In certain embodiments the present invention provides a method of treating a subject having CF comprising administering to the subject an effective amount of a therapeutic agent to alleviate the symptoms of CF, wherein the agent comprises miR-138, a miR-138 mimic, an anti-SIN3A RNAi molecule, and/or an anti-SIN3A antisense oligonucleotide (ASO) or other agent that suppresses SIN3A expression, a small molecule drug that interferes with SIN3A activity or whose actions mimic the biological effects of SIN3A suppression.

In certain embodiments, the present invention provides a method of treating a subject having CF comprising administering to the subject an effective amount of a therapeutic agent to alleviate the symptoms of CF, wherein the agent comprises miR-138, a miR-138 mimic, an anti-SIN3A RNAi molecule, and/or an anti-SIN3A antisense oligonucleotide (ASO) or other agent that suppresses SIN3A expression, a small molecule drug that interferes with SIN3A activity or whose actions mimic the biological effects of SIN3A suppression.

In certain embodiments, the present invention provides a method for increasing chloride ion conductance in airway epithelial cells of a subject afflicted with cystic fibrosis, wherein the subject's CFTR protein has a loss of phenylalanine at position 508, the method comprising administering to the subject a therapeutic agent, wherein the agent comprises miR-138, a miR-138 mimic, an anti-SIN3A RNAi molecule, and/or an anti-SIN3A antisense oligonucleotide (ASO) or other agent that suppresses SIN3A expression, a small molecule drug that interferes with SIN3A activity or whose actions mimic the biological effects of SIN3A suppression. In certain embodiments, the present invention provides a pharmaceutical composition for treatment of cystic fibrosis, comprising miR-138, a miR-138 mimic, an anti-SIN3A RNAi molecule, and/or an anti-SIN3A antisense oligonucleotide (ASO) or other agent that suppresses SIN3A expression, a small molecule drug that interferes with SIN3A activity or whose actions mimic the biological effects of SIN3A suppression in combination with a pharmaceutically acceptable carrier, where the composition does not comprise genistein as an active ingredient, and wherein the composition further comprises a standard cystic fibrosis pharmaceutical, such as an antibiotic.

In certain embodiments, the agent is administered orally or by inhalation. In certain embodiments, the administration is via aerosol, dry powder, bronchoscopic instillation, intra-airway (tracheal or bronchial) aerosol or orally. In certain embodiments, the epithelial cells are intestinal cells, and may be present in a mammal. In certain embodiments, the agent is administered orally.

In certain embodiments, the present invention provides a therapeutic agent comprising miR-138, a miR-138 mimic, an anti-SIN3A RNAi molecule, and/or an anti-SIN3A antisense oligonucleotide (ASO) or other agent that suppresses SIN3A expression, a small molecule drug that interferes with SIN3A activity or whose actions mimic the biological effects of SIN3A suppression for use in treating CF and restoring function to the ΔF508 protein. As used herein the term “restoring function” means that at least 5%-100% of the protein is active. Restored function indicates that the misfolded mutant ΔF508 protein has been rescued from degradation in the proteosome, and successfully trafficked to the cell membrane where it forms a partially functional anion channel. Here it is able to conduct anions such as chloride and bicarbonate. In certain embodiments, the invention provides a pharmaceutical composition for treatment of cystic fibrosis, comprising miR-138, a miR-138 mimic, an anti-SIN3A RNAi molecule, and/or an anti-SIN3A antisense oligonucleotide (ASO) or other agent that suppresses SIN3A expression, a small molecule drug that interferes with SIN3A activity or whose actions mimic the biological effects of SIN3A suppression in combination with a pharmaceutically acceptable carrier, where the composition does not comprise genistein as an active ingredient, and wherein the composition further comprises a CF therapeutic agent.

In certain embodiments, the present invention provides a use of a therapeutic agent comprising miR-138, a miR-138 mimic, an anti-SIN3A RNAi molecule, and/or an anti-SIN3A antisense oligonucleotide (ASO) or other agent that suppresses SIN3A expression, a small molecule drug that interferes with SIN3A activity or whose actions mimic the biological effects of SIN3A suppression to prepare a medicament useful for treating CF in an animal.

In certain embodiments of the methods, pharmaceutical compositions and uses discussed above, the CFTR therapeutic agent is aminoglutethimide, biperiden, diphenhydramine, rottlerin, midodrine, thioridazine, sulfadimethoxine, neostigmine bromide, pyridostigmine, pizotifen, tyrophostin (AG-1478), valproic acid, scriptaid or neomycin.

The present invention further provides a method of substantially restoring CFTR anion channel function in order to provide a therapeutic effect. As used herein the term “substantially restoring” or “substantially restored” refers to increasing the expression of the target gene or target allele by at least about 5%, 10%, 15%, 20%, 25%, 30%, 35%, 40%, 45%, 50%, 55%, 60%, 65%, 70%, 75%, 80%, 85% to 100%. As used herein “increased expression” means that the amount of mRNA is increased, the amount of protein is increased and/or the activity of the protein is increased as compared to CFTRΔF508. As used herein the term “therapeutic effect” refers to a change in the associated abnormalities of the disease state, including pathological and behavioral deficits; a change in the time to progression of the disease state; a reduction, lessening, or alteration of a symptom of the disease; or an improvement in the quality of life of the person afflicted with the disease. Therapeutic effects can be measured quantitatively by a physician or qualitatively by a patient afflicted with the disease state targeted by the therapeutic agent.

### BRIEF DESCRIPTION OF DRAWINGS

FIG. 1: miR-138 and SIN3A regulate CFTR expression in airway epithelia. a, SIN3A mRNA abundance in human primary airway epithelia 24 hrs after indicated interventions (n=6). Scr (negative control), SIN3A DsiRNA (positive control), UnT (Un-transfected cells). b, SIN3A protein abundance in primary airway epithelia 72 hrs post-transfection (representative immunoblot). c, CFTR mRNA abundance in Calu-3 cells 24 hrs after indicated transfections. CFTR DsiRNA (positive control). d, CFTR protein abundance in Calu-3 cells 72 hrs post-transfection (R-769 antibody). Changes in (e) conductance (Gₜ) and (f) transepithelial current (Iₜ) with indicated treatments. All panels: Error bars indicate mean±SE, *P<0.01 relative to Scr, ⁺P<0.01 and ⁺⁺P<0.01 relative to ΔGₜ or ΔIₜ in Scr transfected samples upon forskolin and IBMX (F&I) or CFTR inhibitor GlyH-101 treatment, respectively.

FIG. 2: miR-138 and SIN3A regulate CFTR expression in primary cultures of human airway epithelia and cells with no CFTR expression. a, CFTR mRNA abundance in primary airway epithelia 24 hrs after interventions (n=6). b, CFTR protein abundance from primary airway epithelia 72 hrs post-transfection (R-769 antibody, representative immunoblot). Changes in (c) conductance (Gₜ) and (d) transepithelial current (Iₜ) with indicated treatments. c, d, Each bar represents 6 primary airway epithelial cell cultures each from 3 donors, pre-transfected with reagents noted. e, CFTR protein abundance in HeLa cells (R-769 antibody). f, Schematic representing miR-138 and SIN3A mediated regulation of CFTR expression. g, Fold enrichment of SIN3A, assessed by Q-PCR after chromatin immunoprecipitation. Data normalized to CFTR intron 17a DHS. Inset: CTCF immunoblot of lysates from 3 airway epithelia donors. All panels: Error bars indicate mean±SE; *P<0.01 relative to Scr; ^{**}P<0.01 relative to Int 17a, ⁺P<0.01 and ⁺⁺P<0.01 relative to ΔGₜ or ΔIₜ in Scr transfected samples upon F&I or GlyH-101 treatment, respectively.

FIG. 3: miR-138 regulates CFTR processing. a, Surface display, as detected by ELISA, of epitope tagged CFTR in CFTR-3HA HeLa cells transfected with indicated reagents. b, CFTR protein abundance in CFTR-3HA HeLa cells 24 hrs post-transfection (anti-HA antibody-upper panel, R769 antibody-lower panel). c, Schematic revealing regions of intersection of SIN3A DsiRNA, miRNA-mimic and CFTR-associated genes data sets, P<0.05 (See Tables 2-4). d, Surface display of epitope tagged CFTR in CFTR-ΔF508-3HA HeLa cells transfected with indicated reagents. e, CFTR protein abundance in CFTR-ΔF508-3HA HeLa cells 24 hrs post-transfection (anti-HA antibody-upper panel, R769 antibody-lower panel). All panels: Error bars indicate mean±SE; *P<0.01 relative to Scr.

FIG. 4: SIN3A inhibition yields partial rescue of cl⁻ transport in CF epithelia. a, Upper panel: CFTR protein abundance from airway epithelia (CFTR Q493X/S912X, 24-1 antibody) following indicated treatments. Lower panel: Iₜ following F&I stimulation and GyH-101 inhibition. n=1 donor, 3 replicates. b, Representative CFTR immunoblot from primary epithelia (CFTR ΔF508/ΔF508) 72 hrs post-transfection (R-769 antibody, Donor #1 in FIG. 4c). c, Responses of CFTR ΔF508/ΔF508 epithelia to indicated interventions (Donor #1 in FIG. 4c). Upper panel: Iₜ tracings of responses to F&I, followed by GlyH-101 treatment (epithelia pretreated with amiloride and DIDS). Lower panel: Summary of change in Iₜ in response to F&I, followed by GlyH-101 treatment. n=1 donor, 8 replicates. (a & c) Error bars indicate mean±SE, *P<0.01 and **P<0.01 relative to ΔIₜ in Scr transfected samples following F&I or GlyH-101 treatments, respectively, ⁺P<0.01 relative to Scr. d, Change in Iₜ following F&I treatment of 6 primary CF airway epithelia cultures transfected with indicated reagents. 6 untreated or Scr treated CF samples provide negative controls. 8 non-CF samples provide wild-type controls. ΔF/* denotes ΔF508/3659delC, ΔF/** denotes ΔF508/R1162X. Horizontal bars indicate mean. e, Working model of steps in CFTR transcription and protein biosynthetic pathway where miR-138-regulated gene products influence wild-type and CFTR-ΔF508 (See FIG. 3c, Tables 2-4).

FIG. 5: miR-138 regulates SIN3A in a dose-dependent and site-specific manner. HEK293T cells were co-transfected with the psiCHECK-2 vector (containing the SIN3A 3′UTR) and increasing concentrations of Scr or miR-138 mimic (Scr: non-targeting control oligonucleotide). To test site-specificity, the two predicted binding sites of miR-138 on SIN3A 3′UTR cloned in the psiCHECK-2 vector were mutated and the experiment repeated. Error bars indicate mean±SE; (n=4, 3 replicates each); *P<0.01, relative to Scr.

FIG. 6: miR-138 regulates endogenous SIN3A protein expression. Densitometry and relative fold change of SIN3A protein abundance in 6 human donors of primary airway epithelial cultures (8 replicates each). Immunoblots were performed 72 hrs post-transfection. SIN3A DsiRNA (positive control), UnT (Un-transfected cells). Error bars indicate mean±SE, *P<0.01, relative to Scr.

FIG. 7: miR-138 regulates endogenous CFTR protein expression in Calu-3 cells. a, Representative CFTR immunoblot in Calu-3 cells 72 hrs post-transfection. PVDF membrane was first probed with R769 antibody (shown in FIG. 2b), stripped and re-probed with the M3A7+MM13-4 antibody cocktail. b, Densitometry and relative fold change of CFTR protein abundance (R769 antibody) from (n=4, 3 replicates each). Error bars indicate mean±SE, ^{#}P<0.01, relative to Scr CFTR band B; ^{##}P<0.01, relative to Scr CFTR band C.

FIG. 8: miR-138 regulates endogenous CFTR protein expression in primary human airway epithelia. a, CFTR immunoblot from one human donor of primary airway epithelial 72 hrs post-transfection. PVDF membrane was first probed with R769 antibody (shown in FIG. 1d), stripped and re-probed with the M3A7+MM13-4 antibody cocktail. b, Densitometry and relative fold change of CFTR protein abundance (R769 antibody) in primary, airway epithelia from 6 different human donors (8 replicates each). Error bars indicate mean±SE, ^{#}P<0.01, relative to Scr CFTR Band B; ^{##}P<0.01, relative to Scr CFTR Band C.

FIG. 9: miR-138 regulates CFTR expression in HeLa cells. a, Relative CFTR and SIN3A mRNA abundance in HeLa cells 24 hrs post-transfection (n=4, 8 replicates each). b, Representative CFTR immunoblot (n=4, 3 replicates each) performed 72 hrs post-transfection. PVDF membrane was first probed with R769 antibody (shown in FIG. 2e), stripped and re-probed with the M3A7+MM13-4 antibody cocktail. Densitometry not shown as no CFTR protein detected in HeLa cells. Error bars indicate mean±SE, *P<0.01, relative to Scr (for CFTR); **P<0.01, relative to Scr (for SIN3A).

FIG. 10: miR-138 regulates CFTR expression in HEK293T cells. a, Relative CFTR and SIN3A mRNA abundance in HEK293T cells 24 hrs post-transfection (n=4, 8 replicates each). b, Representative CFTR immunoblots (done in triplicate from 4 separate experiments) performed 72 hrs post-transfection. PVDF membrane was first probed with R769 antibody (top panel), stripped and re-probed with the M3A7+MM13-4 antibody cocktail (bottom panel). Densitometry not shown as no CFTR protein detected in HEK293T cells. Error bars indicate mean±SE, *P<0.01, relative to Scr (CFTR); **P<0.01, relative to Scr (SIN3A).

FIG. 11: HeLa cells exhibit CFTR channel activity. a, b, Iodide efflux assay performed in HeLa cells 48 hrs post-transfection with the miR-138 mimic and SIN3A DsiRNA (8 independent transfections per condition). HeLa cells stably expressing the wild-type CFTR (CFTR-3HA-HeLa) were used as controls. Each data point represents 8 transfections. ⁺P<0.01. F&I denotes addition of forskolin and IBMX as described in Methods.

FIG. 12: miR-138 improves CFTR processing. a, Cell surface ELISA to detect CFTR with an anti-HA antibody in HeLa-CFTR cells 6, 12, and 24 hrs post-transfection with noted reagents (n=3, 6 replicates each). b, Relative CFTR mRNA abundance in Hela-CFTR cells 24 hrs post-transfection. Primers were designed to distinguish between endogenous CFTR mRNA and the CFTR-HA transgene (n=3, 6 replicates each). c, d, Densitometry and relative fold change of CFTR protein abundance (n=4, 8 replicates each) in HeLa cells stably expressing the wild type CFTR-3HA. c, Anti-HA antibody (Covance). d, Anti-CFTR antibody (R769 antibody). Based on results in HeLa cells (FIG. 2e, FIG. 9) and the increase in endogenous CFTR mRNA (FIG. 12b) in response to miR-138 mimic or SIN3A DsiRNA, the increased abundance of CFTR band C represents the sum of both CFTR-3HA biogenesis and endogenous CFTR protein expression. Error bars indicate mean±SE; *P<0.01 relative to Scr; ^{#}P<0.01, relative to Scr CFTR band B; ^{##}P<0.01, relative to Scr CFTR band C.

FIG. 13: miR-138 improves CFTR-ΔF508 processing. a, Cell surface ELISA to detect CFTR-ΔF508 with an anti-HA antibody in HeLa-CFTR-ΔF508 cells 6, 12 and 24 hrs post-transfection with noted reagents (n=3, 6 replicates each). b, Relative CFTR mRNA abundance in Hela-CFTR cells 24 hrs post-transfection. Primers were designed to distinguish between endogenous CFTR mRNA and the CFTR-HA transgene (n=3, 6 replicates each). c, d, Densitometry and relative fold change of CFTR-ΔF508 protein abundance (n=4, 8 replicates each) in HeLa cells stably expressing HA-tagged CFTR-ΔF508. Fold change of band C not shown, as no band C detected in Scr and UnT samples. c, Anti-HA antibody (Covance). d, Anti-CFTR antibody (R769 antibody). Based on results in HeLa cells (FIG. 2e, FIG. 9) and the increase in endogenous CFTR mRNA (FIG. 12b) in response to miR-138 mimic or SIN3A DsiRNA, the increased abundance of CFTR band C represents the sum of both the increased abundance of HA-tagged CFTR-ΔF508 processing as well as endogenous CFTR protein expression. Error bars indicate mean±SE; *P<0.01 relative to Scr; ^{#}P<0.01, relative to Scr CFTR band B.

FIG. 14: SIN3A inhibition yields partial rescue of Cl⁻ transport in CF epithelia. a, Representative tracings of transepithelial current (Iₜ) responses after sequential apical application of noted reagents in primary CFTR null human airway epithelial (CFTR Q493X/S912X). b, Average transepithelial current (Iₜ) responses after sequential apical application of noted reagents in primary airway epithelia (CFTR Q493X/S912X). Aml=Amiloride. Each data point represented by 3 cultures. Error bars indicate mean±SE, *P<0.01, relative to Scr (SIN3A); **P<0.01, relative to Scr after F&I stimulation.

FIG. 15: miR-138 regulates endogenous CFTR and SIN3A expression in CF primary airway epithelia. Relative CFTR-ΔF508 and SIN3A mRNA abundance in 4 human donors of CF (ΔF508/ΔF508) primary airway epithelia 24 hrs post-transfection (8 replicates per donor). Error bars indicate mean±SE, *P<0.01, relative to Scr (for CFTR); **P<0.01, relative to Scr (for SIN3A).

FIG. 16: SIN3A inhibition yields partial rescue of Cl⁻ transport in CF epithelia. a, CFTR-ΔF508 immunoblot in a human donor of primary CF (ΔF508/ΔF508) primary airway epithelia 72 hrs post-transfection (8 replicates, Donor #1 on FIG. 4d). PVDF membrane was first probed with R769 antibody (shown in FIG. 4b), stripped and re-probed with the M3A7+MM13-4 antibody cocktail. b, Representative tracings of transepithelial current (Iₜ) response after sequential apical application of noted reagents in primary airway epithelia (CFTR ΔF508/ΔF508). c, Average transepithelial current (Iₜ) responses after sequential apical application of noted reagents. Each data point represented by 8 cultures. Error bars indicate mean±SE, *P<0.01 relative to Scr after F&I stimulation.

FIG. 17: miR-138 regulates endogenous CFTR and SIN3A expression in CFBE cells. a, Relative CFTR-ΔF508 and SIN3A mRNA abundance in CFBE cells (CFTR ΔF508/ΔF508) 24 hrs post-transfection (n=4, 8 replicates). b, Representative CFTR immunoblot in CFBE cells performed 72 hrs post-transfection (n=4, 8 replicates). PVDF membrane was first probed with R769 antibody (top panel), stripped and re-probed with the M3A7+MM13-4 antibody cocktail (bottom panel). c, Representative tracings of transepithelial current (Iₜ) response after sequential apical application of noted reagents in CFBE cells (CFTR ΔF508/ΔF508). d, Average transepithelial current (Iₜ) responses after sequential apical application of noted reagents. Each data point represented by 8 CFBE ALI cultures. e, Change in transepithelial current (ΔIₜ) after stimulation with Forskolin+IBMX (F&I) and GlyH. Each data point represented by 8 CFBE ALI cultures. All panels, Error bars indicate mean±SE. *P<0.01, relative to Scr (CFTR); **P<0.01, relative to Scr (SIN3A); ^{#}P<0.01 relative to Iₜ in Scr transfected samples upon F&I addition; ⁺P<0.01 and ⁺⁺P<0.01 relative to ΔIₜ in Scr transfected samples upon F&I or GlyH-101 stimulation respectively.

FIG. 18: Specificity of oligonucleotide transfections. Relative expression by RT-qPCR of GAPDH and HPRT (normalized to SFRS9), and miRs-21, -24, -26a, -200c, -146a, -146b, -27a*, -134 (normalized to RNU48). Experiment performed 24 hrs post-transfection in a, Primary airway epithelia from human non-CF donor #1 (6 replicates), b, Primary airway epithelia from human non-CF donor #2 (6 replicates), c, Primary airway epithelia from human non-CF donor #3 (6 replicates), d, Calu-3 cells (n=4, 6 replicates each), e, HEK293T cells (n=4, 6 replicates each), f, HeLa cells (n=4, 6 replicates each), and g, CFBE (CFTR ΔF508/ΔF508) cells (n=4, 6 replicates each). All panels, Error bars indicate mean±SE. UnD=Undetected by RT-qPCR.

FIG. 19: Persistence of oligonucleotide effects 2 weeks post-transfection. a, Representative SIN3A immunoblot in Calu-3 air-liquid interface (ALI) cultures 14 days post-transfection (6 replicates). b, Relative SIN3A mRNA abundance in Calu-3 ALI cultures 14 days post transfection (6 replicates). c, Representative CFTR immunoblot in Calu-3 ALI cultures 14 days post-transfection (8 replicates). PVDF membrane was first probed with R769 antibody (top panel), stripped and re-probed with the M3A7+MM13-4 antibody cocktail (bottom panel). d, Relative CFTR mRNA abundance in Calu-3 ALI cultures 14 days post transfection (6 replicates). e, Representative CFTR immunoblot in CFBE (CFTR ΔF508/ΔF508) ALI cultures 14 days post-transfection (6 replicates). PVDF membrane was first probed with R769 antibody (top panel), stripped and re-probed with the M3A7+MM13-4 antibody cocktail (bottom panel). f, Relative CFTR mRNA abundance in CFBE ALI cultures 14 days post-transfection (6 replicates). All panels, Error bars indicate mean±SE. *P<0.01, relative to Scr.

FIG. 20: Effects of drugs identified from CMAP screen on DF508 trafficking to the cell membrane. HeLa cells stably expressing DeltaF508 with an HA tag were treated with the indicated compounds for 96 hr. Following treatment, cells were processed for cell surface ELISA using an anti-HA antibody. Results show that several compounds increase DF508 processing and surface display. C4 indicates Corrector 4, a small molecule known to enhance DeltaF508 processing. Drug concentrations used (micro-moles/liter); Valproic Acid 50, Thioridazine 0.1, Tyrophostine AG-1478 3.2, Rottlerin 1, Pizotifen 9, Neomycin 4, Neostigmine bromide 13, MidodrineHCl 14, Diphenhydramine 14, Sulfadimethoxine 13, Scriptaid 10, Biperiden 11, H7 1, Aminoglutethimide 17, Pyridostigmine 15. DMSO at 1:1000 dilution. * indicates P<0.05. N=4 replicates/condition.

FIG. 21. RNA interference screen identifies candidate genes involved in the rescue of ΔF508-CFTR trafficking. Relative surface display of ΔF508-CFTR measured by live cell-surface ELISA using an anti-HA antibody performed 72 hr post-transfection. HeLa-ΔF508-CFTR-HA cells were transfected with 100 nM of DsiRNAs against each gene. Black bars: genes whose knockdown rescued ΔF508-CFTR trafficking efficiently with both DsiRNAs; Grey bars: genes whose knockdown rescued ΔF508-CFTR trafficking with at least one DsiRNAs. Each bar represents fold increase relative to the Scrambled (Scr) transfection; 24 transfections per DsiRNA from 4 separate experiments; 2 separate DsiRNAs per gene. Gene IDs are provided in Table 6. Statistical significance calculated by Student's t-test, *P value<0.05, **P value<0.01, ***P value<0.001.

FIGS. 22A and 22B. RNA interference screen identifies candidate genes involved in the rescue of ΔF508-CFTR maturation. (A) Representative blot depicting ΔF508-CFTR expression in CFBE 41o⁻ cells (homozygous for ΔF508-CFTR). Each lane represents protein harvested from 2 separate transfections; DsiRNAs against each gene were transfected at a final concentration of 100 nM. Protein was harvested 72 hr post-transfection. (B) Densitometry representing fold increase of ΔF508-CFTR band C and ΔF508-CFTR band B in CFBE 41o⁻ cells transfected with two separate DsiRNAs against each genes at a final concentration of 100 nM. Data generated from three experiments. Gene IDs are provided in Table 6. Statistical significance calculated by Student's t-test, *P value<0.05, **P value<0.01, ***P value<0.001.

FIG. 23. RNA interference screen identifies 4 candidate genes involved in the rescue of ΔF508-CFTR trafficking. Relative surface display of ΔF508-CFTR measured by live cell-surface ELISA using an anti-HA antibody performed 72 hr post-transfection. HeLa-ΔF508-CFTR-HA cells were transfected with 100 nM of DsiRNAs against selected genes from FIG. 1. Black bars: genes whose knockdown rescued ΔF508-CFTR trafficking efficiently with both DsiRNAs. Each bar represents fold increase relative to the Scrambled (Scr) transfection; 18 transfections per DsiRNA from 3 separate experiments; 2 separate DsiRNAs per gene. Gene IDs are provided in Table 6. Statistical significance calculated by Student's t-test, *P value<0.05, **P value<0.01, ***P value<0.001.

FIG. 24. Individual and combinatorial repression of candidate genes by RNA interference. Relative surface display of ΔF508-CFTR measured by live cell-surface ELISA using an anti-HA antibody performed 72 hr post-transfection. HeLa-ΔF508-CFTR-HA cells were transfected with DsiRNAs at a final concentration of 100 nM, targeting either 1 or more candidate genes. Each bar represents fold change relative to the Scrambled (Scr) transfection; 30 transfections per gene/gene combination from 5 separate experiments. Gene IDs: 6-NHERF1, 7-CAPNS1, 11-HSP90B1, 15-SYVN1, 17-RCN2. Statistical significance calculated by Student's t-test, *P value<0.05, **P value<0.01, ***P value<0.001.

FIGS. 25A and 25B. SYVN1 knockdown significantly rescues ΔF508-CFTR maturation in CFBE cells. (A) Representative blot depicting ΔF508-CFTR expression in CFBE 41o⁻ cells (homozygous for ΔF508-CFTR). Each lane represents protein from 2 separate transfections, DsiRNAs were transfected at a final concentration of 100 nM. Protein was harvested 72 hr post-transfection. (B) Densitometry representing fold increase of ΔF508-CFTR band C and ΔF508-CFTR band B in CFBE 41o⁻ cells transfected with DsiRNAs against each gene/gene combination at a final concentration of 100 nM. Data generated from 6 experiments. Gene IDs: 6-NHERF1, 7-CAPNS1, 11-HSP90B1, 15-SYVN1, 17-RCN2. Statistical significance calculated by Student's t-test, *P value<0.05, **P value<0.01, ***P value<0.001.

FIGS. 26A and 26B. SYVN1 knockdown significantly rescues ΔF508-CFTR mediated Cl⁻ transport in CFBE cells. (A) Change in Iₜ following F&I treatment of CFBE 41o⁻ cells with indicated reagents. 22 Scr and 6 NoT (no treatment) cultures provide negative controls. C18 (corrector compound) and SIN3A knockdown provide positive controls. Horizontal bars indicate mean. Statistical significance calculated by Student's t-test, **P value<0.01, ***P value<0.001. (B) Representative tracings of transepithelial current (Iₜ) responses after sequential apical application of indicated reagents in CFBE 41o⁻ cells. Time of addition of reagents is indicated by arrows.

FIG. 27. SYVN1 knockdown significantly rescues ΔF508-CFTR mediated Cl⁻ transport in primary CF airway epithelia. Change in Iₜ following F&I+PG-01 treatment of primary CF airway epithelial cells (homozygous for ΔF508-CFTR) with indicated reagents. N=1 donor. PG-01 is a potentiator used along with F&I to increase Cl⁻ transport.

FIG. 28. 5 drugs consistently rescue ΔF508-CFTR trafficking. Relative surface display of ΔF508-CFTR measured by live cell-surface ELISA using an anti-HA antibody performed 72 hr post-treatment. HeLa-ΔF508-CFTR-HA cells were treated daily with the mentioned drugs at the indicated concentrations. DMSO is the vehicle control, C4a is a small molecule CFTR corrector compound. Statistical significance calculated by Student's t-test, *P value<0.05, **P value<0.01, ***P value<0.001.

FIG. 29. Pyridostigmine rescues ΔF508-CFTR trafficking. Relative surface display of ΔF508-CFTR measured by live cell-surface ELISA using an anti-HA antibody performed 72 hr post-treatment. HeLa-ΔF508-CFTR-HA cells were treated daily with the mentioned drugs. Concentrations used were as follows: Pyridosigmine (Py)-15 μM, Biperiden (Bi)-11 Tyrphostin (Tyr)-0.03 μM, Pizotifen (Pizo)-9 μM. DMSO is the vehicle control, C18 and C4a are small molecule CFTR corrector compounds used as positive controls. Statistical significance calculated by Student's t-test, *P value<0.05, **P value<0.01, ***P value<0.001.

FIG. 30. Combination of Pyridostigmine and Biperiden rescues ΔF508-CFTR maturation and trafficking. (A) Representative blot depicting ΔF508-CFTR expression in CFBE 41o⁻ cells (homozygous for ΔF508-CFTR) treated daily with the mentioned drugs (Py-pyridostigmine, Bi-biperiden) at the indicated concentrations (μM). Protein was harvested 72 hr post-treatment. (B) Densitometry representing fold increase of ΔF508-CFTR band C and ΔF508-CFTR band B in CFBE 41o⁻ cells relative to DMSO. Data generated from 8 immunoblot experiments. Statistical significance calculated by Student's t-test, *P value<0.05, **P value<0.01, ***P value<0.001.

Table 1: Expression of microRNAs in human airway epithelia. AB TaqMane Low Density MicroRNA Array (TLDA) was performed on 4 human non-CF primary well-differentiated airway epithelial cultures. With a C_{q} cut-off<30, 115 miRNAs were deemed expressed in the human airway epithelium. Of these, 31 miRNAs (bold) were highly expressed with an average C_{q}value<25. MiRNAs arranged in order of their decreasing average abundance.

Table 2: CFTR-Associated Gene Network. This gene list was curated from the published literature and includes gene products as identified as directly or indirectly involved in CFTR biosynthesis (Wang, X. et al. Hsp90 cochaperone Aha1 downregulation rescues misfolding of CFTR in cystic fibrosis. Cell 127, 803-815 (2006); Okiyoneda, T. et al. Peripheral protein quality control removes unfolded CFTR from the plasma membrane. Science 329, 805-810 (2010); Hutt, D. M. et al. Reduced histone deacetylase 7 activity restores function to misfolded CFTR in cystic fibrosis. Nature Chem. Biol. 6, 25-33 (2010); Liekens, A. M. et al. BioGraph: unsupervised biomedical knowledge discovery via automated hypothesis generation. Genome Biol. 12, R57 (2011); Gomes-Alves, P., Neves, S., Coelho, A. V. & Penque, D. Low temperature restoring effect on F508del-CFTR misprocessing: A proteomic approach. J Proteomics 73, 218-230 (2009)).

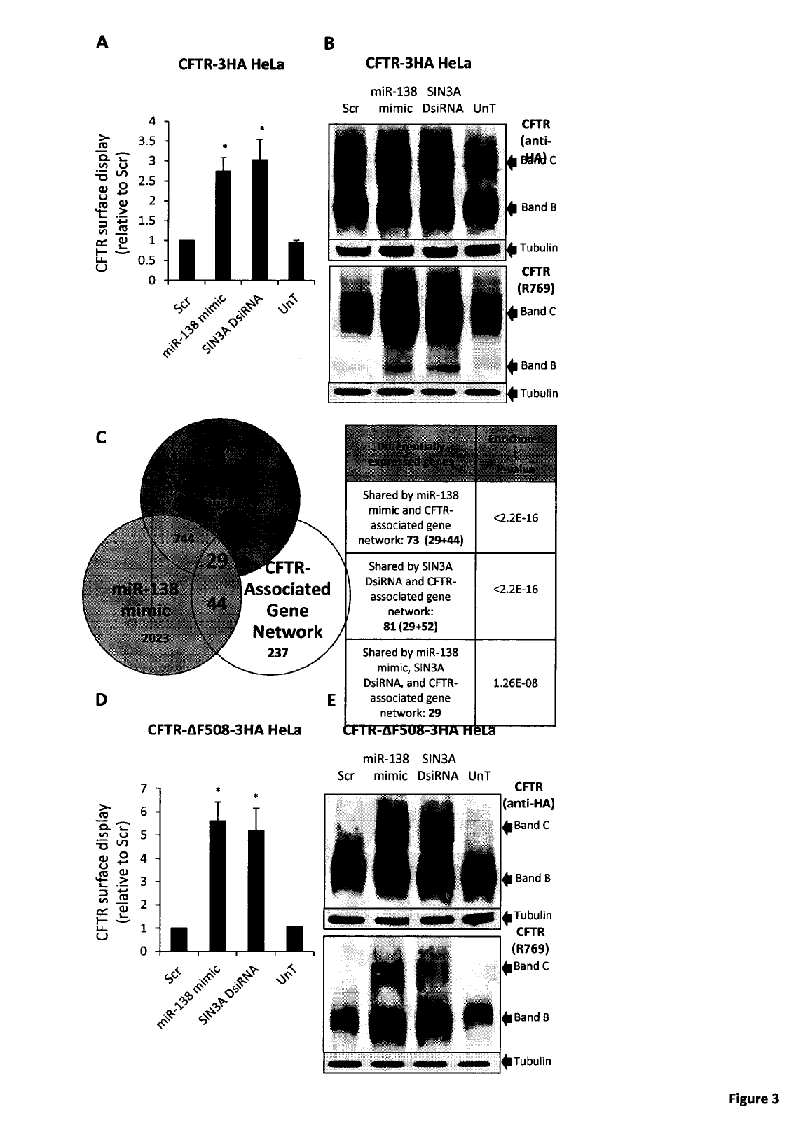

Table 3: Enrichment significance for genes influencing CFTR biogenesis. Differentially expressed genes from the miR-138 mimic or SIN3A DsiRNA microarray experiment in Calu-3 cells were cross-referenced with the CFTR-Associated Gene Network (FIG. 3c). Fisher's Exact Test was used to generate an enrichment score for genes in the CFTR-Associated Gene Network from either one or both array datasets and referenced against the background (expressed genes with fold change <1.5 and P value>0.05).

Table 4: Genes in the CFTR-Associated Gene Network identified as differentially expressed in Calu-3 cells following miR-138 or SIN3A DsiRNA treatment. The 125 genes in the CFTR-Associated Gene Network identified as differentially expressed in Calu-3 cells following treatment with SIN3A DsiRNA, miR-138 mimic, or negative control (Scr) (FIG. 3c). RNA was isolated from Calu-3 cells 48 hrs post-transfection for each experiment. The cellular compartments where each gene product has been indicated to function are indicated. The BOLD text indicates the 29 differentially expressed genes (FIG. 3c, Table 3) found by intersecting the SIN3A DsiRNA array, miR-138 mimic array, and the CFTR-Associated Gene Network. Italicized text indicates the 52 differentially expressed genes (FIG. 3c, Table 3) identified by intersecting the SIN3A DsiRNA array and the CFTR-Associated gene network. The remaining, unmarked text denotes the 44 differentially expressed genes (FIG. 3c, Table 3) found by intersecting the miR-138 mimic array and the CFTR-Associated Gene Network. A literature survey identified that several of the differentially expressed gene products are known to influence CFTR protein biogenesis (references indicated).

Table 5: List of representative miR-138 molecules.

Table 6. Genes included in the RNA interference screen. 125 genes known to associate with CFTR and respond to miR-138 mimic or SIN3A DsiRNA interventions were identified (Ramachandran et al., Proc Natl Acad Sci USA. 2012 Aug. 14; 109(33):13362-7). These genes function in several cellular compartments and 25 genes were picked for an RNA interference screen whose loss of expression was most likely to positively influence CFTR protein expression or stability.

### DETAILED DESCRIPTION OF THE INVENTION

In certain embodiments, the present invention provides methods of using therapeutic agents to treat cystic fibrosis.

The present technology is based on a new discovery concerning the pathways for controlling CFTR gene expression and protein biogenesis. The inventors have found that SIN3A plays a crucial role in the expression of the CFTR gene. SIN3A does this by associating with the CTCF protein (transcriptional repressor recognizing CCCTC) and then binding the promoter for the CFTR gene resulting in transcriptional inhibition. The inventors also discovered that miR-138 suppresses the SIN3A transcript by blocking its translation. SIN3A is a significant target of miR-138 and plays a critical role in the pathophysiology of CF. Combining these findings, miR-138 can be used therapeutically to inhibit SIN3A, a key component in the inhibition of CFTR transcription, thus increasing CFTR transcription rates. In addition, the inventors show that miR-138 and SIN3A regulate a gene network in airway epithelia. Therapeutic manipulation of this gene network contributes to restoring function to the mutant protein by improving protein processing.

The inventors have found that the increase in CFTR protein production in CF cells that are homozygous or heterozygous for the ΔF508 mutation is enough to overcome the systematic degradation of those imperfect proteins, allowing some of those proteins to take their place in the outer cell membrane and provide enough channel function to alleviate the effects of the disease. This result assumes that the mutant CFTR protein is still able to serve some anion channel function, which the inventors have confirmed with their findings.

The next aspect of this invention involved the use of the miR-138 and SIN3A data along with a “connectivity map” software program to identify candidate chemical agents that have been associated with a similar transcriptional control profile (increase in miR-138 activity or decrease in SIN3A expression). Using this process, the inventors identified a candidate pool of known/commercialized chemical entities to further screen for a CFTR-targeted therapy. These candidate agents include Aminoglutethimide, Biperiden, diphenhydramine, Rottlerin, Midodrine, Thioridazine, Sulfadimethoxine, neostigmine bromide, Pyridostigmine, pizotifen, tyrophostin (AG-1478), valproic acid, Scriptaid or neomycin.

Therapeutic Agents

1. pre-miR-138 and miR-138:

Pre-miR-138:
hsa-mir-138-1 MI0000476
(SEQ ID NO: 1)
CCCUGGCAUGGUGUGGUGGGGCAGCUGGUGUUGUGAAUCAGGCCGUUGCC
AAUCAGAGAACGGCUACUUCACAACACCAGGGCCACACCACACUACAGG
hsa-mir-138-2 MI0000455
(SEQ ID NO: 2)
CGUUGCUGCAGCUGGUGUUGUGAAUCAGGCCGACGAGCAGCGCAUCCUCU
UACCCGGCUAUUUCACGACACCAGGGUUGCAUCA
Mature miRNA:
hsa-mir-138-5p
(SEQ ID NO: 3)
AGCUGGUGUUGUGAAUCAGGCCG
miR-138 mimic -
Sense strand sequence:
(SEQ ID NO: 4)
/5SpC3/rCmG rGmC/iSpC3/ mUrGmA rUmUrC mArCmA
rAmCrA mCrCmA rGmCrU
Antisense strand sequence:
(SEQ ID NO: 5)
/5Phos/rArG rCrUrG rGrUrG rUrUrG rUrGrA rArUrC
rArGrG mCmCmG

As used herein “5SpC3” and “iSpC3” represent propanediol groups (e.g., a “C3 spacer”), rN represent RNA bases, mN represent 2′OMe RNA bases, and 5Phos represents a 5′-phosphate group. For example, as used herein, the designation “ACGU” and “rA rC rG rU” are equivalent. In certain embodiments, a miR-138 mimic is a synthetic nucleic acid which shows miR-138-like activity in a mammalian cell following transfection. In certain embodiments this is a long pri-miRNA, a shorter pre-miRNA (as shown above), the even shorter mature miRNA, or a modified compound which has been optimized to improve performance (as shown above). Many different miR mimics can be designed. The one above was employed in the present studies and is suitable for use as an example but in no way should be restrictive of the wider body of nucleic acid compositions that can be employed as a miR-138 mimic.

CFTR Small Molecule Therapeutic Agents

2. Aminoglutethimide:
(RS)-3-(4-aminophenyl)-3-ethyl-piperidine-2,6-dione

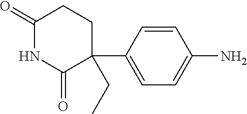 [SMILES: CCC1(c2ccc(N)cc2)CCC(=O)NC1=O]

3. Biperiden:
(1RS,2SR,4RS)-1-(bicyclo[2.2.1]hept-5-en-2-yl)-1-phenyl-3-(piperidin-1-yl)propan-1-ol

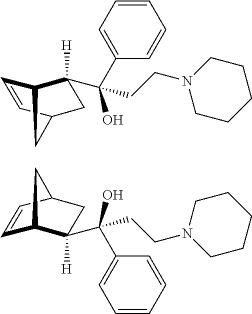

4. Diphenhydramine

5. Rottlerin:
3′-[(8-Cinnamoyl-5,7-dihydroxy-2,2-dimethyl-2H-1-benzopyran-6-yl)methyl]-2′,4′,6′-trihydroxy-5′-methylacetophenone

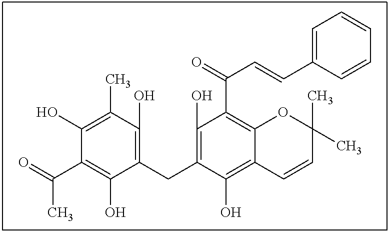 [SMILES: CC(=O)c1c(O)c(C)c(O)c(Cc2c(O)c3c(c(C(=O)/C=C/c4ccccc4)c2O)OC(C)(C)C=C3)c1O]

6. Midodrine:
(RS)— N-[2-(2,5-dimethoxyphenyl)-2-hydroxyethyl]glycinamide

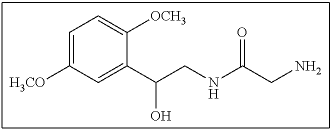 [SMILES: COc1ccc(OC)c(C(O)CNC(=O)CN)c1]

7. Thioridazine:

10-{2-[(RS)-1-Methylpiperidin-2-yl]ethyl}-2-methylsulfanylphenothiazine

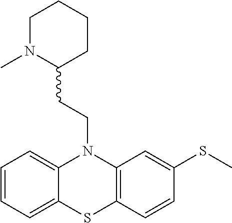

8. Sulfadimethoxine:
4-amino-N-(2,6-dimethoxypyrimidin-4-yl)benzenesulfonamide

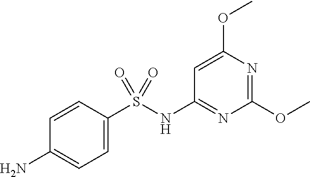 [SMILES: COc1cc(NS(=O)(=O)c2ccc(N)cc2)nc(OC)n1]

9. Neostigmine:
3-{[(dimethylamino)carbonyl]oxy}-N,N,N-trimethylbenzenaminium

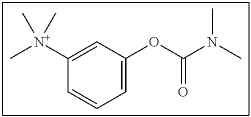

10. Pyridostigmine:
3-[(dimethylcarbamoyDoxy]-1-methylpyridinium

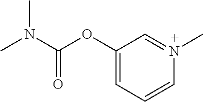

11. Pizotifen:
4-(1-methyl-4-piperidylidine)-9, 1 0-dihydro-4H-benzo-[4,5]cyclohepta[1,2]-thiophene

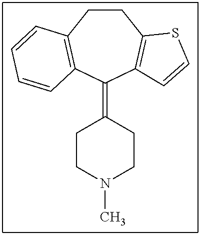 [SMILES: CN1CCC(=C2c3ccccc3CCc3sccc32)CC1]

12. Tyrophostin (AG-1478):
N-(3-chlorophenyl)-6,7-dimethoxy-4-quinazolinamine

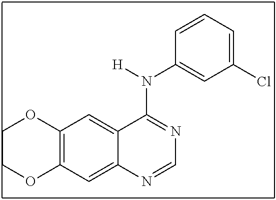 [SMILES: Clc1cccc(Nc2ncnc3cc4c(cc23)OCCO4)c1]

13. Valproic Acid:
2-propylpentanoic acid

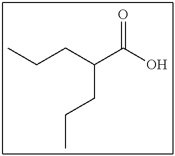

14. Scriptaid:
N-Hydroxy-1,3-dioxo-1H-benz[de]isoquinoline-2(3H)-hexanamide

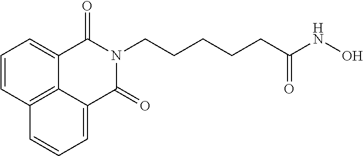 [SMILES: O=C(CCCCCN1C(=O)c2cccc3cccc(c23)C1=O)NO]

15. Neomycin:
O-2,6-diamino-2,6-dideoxy-α-D-glucopyranosyl(1→3)-O-β-D-ribofuranosyl-(1→5) O-[2,6-diamino-2,6-dideoxy-α-D-glucopyranosyl-(1→4)]-2-deoxy-D-streptamine

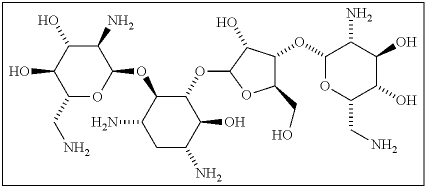

In certain embodiments, pharmaceutically acceptable salts of these compounds are used. For in vivo use, a therapeutic compound as described herein is generally incorporated into a pharmaceutical composition prior to administration. Within such compositions, one or more therapeutic compounds as described herein are present as active ingredient(s) (i.e., are present at levels sufficient to provide a statistically significant effect on the symptoms of cystic fibrosis, as measured using a representative assay). A pharmaceutical composition comprises one or more such compounds in combination with any pharmaceutically acceptable carrier(s) known to those skilled in the art to be suitable for the particular mode of administration. In addition, other pharmaceutically active ingredients (including other therapeutic agents) may, but need not, be present within the composition.

### RNA Interference (RNAi) Molecules

“RNA interference (RNAi)” is the process of sequence-specific, post-transcriptional gene silencing initiated by a small interfering RNA (siRNA). During RNAi, siRNA induces degradation of target mRNA with consequent sequence-specific inhibition of gene expression.

An “RNA interference,” “RNAi,” “small interfering RNA” or “short interfering RNA” or “siRNA” or “short hairpin RNA” or “shRNA” molecule, or “miRNA” is a RNA duplex of nucleotides that is targeted to a nucleic acid sequence of interest, for example, SIN3A. As used herein, the term “siRNA” is a generic term that encompasses all possible RNAi triggers. An “RNA duplex” refers to the structure formed by the complementary pairing between two regions of a RNA molecule. siRNA is “targeted” to a gene in that the nucleotide sequence of the duplex portion of the siRNA is complementary to a nucleotide sequence of the targeted gene. In certain embodiments, the siRNAs are targeted to the sequence encoding SIN3A. In some embodiments, the length of the duplex of siRNAs is less than 30 base pairs. In some embodiments, the duplex can be 32, 31, 30, 29, 28, 27, 26, 25, 24, 23, 22, 21, 20, 19, 18, 17, 16, 15, 14, 13, 12, 11 or 10 base pairs in length. In some embodiments, the length of the duplex is 19 to 32 base pairs in length. In certain embodiment, the length of the duplex is 19 or 21 base pairs in length. The RNA duplex portion of the siRNA can be part of a hairpin structure. In addition to the duplex portion, the hairpin structure may contain a loop portion positioned between the two sequences that form the duplex. The loop can vary in length. In some embodiments the loop is 5, 6, 7, 8, 9, 10, 11, 12, 13, 14, 15, 16, 17, 18, 19, 20, 21, 22, 23, 24, 25, 26 or 27 nucleotides in length. In certain embodiments, the loop is 18 nucleotides in length. The hairpin structure can also contain 3′ and/or 5′ overhang portions. In some embodiments, the overhang is a 3′ and/or a 5′ overhang 0, 1, 2, 3, 4 or 5 nucleotides in length.

As used herein, Dicer-substrate RNAs (DsiRNAs) are chemically synthesized asymmetric 25-mer/27-mer duplex RNAs that have increased potency in RNA interference compared to traditional siRNAs. Traditional 21-mer siRNAs are designed to mimic Dicer products and therefore bypass interaction with the enzyme Dicer. Dicer has been recently shown to be a component of RISC and involved with entry of the siRNA duplex into RISC. Dicer-substrate siRNAs are designed to be optimally processed by Dicer and show increased potency by engaging this natural processing pathway. Using this approach, sustained knockdown has been regularly achieved using sub-nanomolar concentrations. (U.S. Pat. No. 8,084,599; Kim et al., Nature Biotechnology 23:222 2005; Rose et al., Nucleic Acids Res., 33:4140 2005).

The transcriptional unit of a “shRNA” is comprised of sense and antisense sequences connected by a loop of unpaired nucleotides. shRNAs are exported from the nucleus by Exportin-5, and once in the cytoplasm, are processed by Dicer to generate functional siRNAs “miRNAs” stem-loops are comprised of sense and antisense sequences connected by a loop of unpaired nucleotides typically expressed as part of larger primary transcripts (pri-miRNAs), which are excised by the Drosha-DGCR8 complex generating intermediates known as pre-miRNAs, which are subsequently exported from the nucleus by Exportin-5, and once in the cytoplasm, are processed by Dicer to generate functional miRNAs or siRNAs. “Artificial miRNA” or an “artificial miRNA shuttle vector”, as used herein interchangably, refers to a primary miRNA transcript that has had a region of the duplex stem loop (at least about 9-20 nucleotides) which is excised via Drosha and Dicer processing replaced with the siRNA sequences for the target gene while retaining the structural elements within the stem loop necessary for effective Drosha processing. The term “artificial” arises from the fact the flanking sequences (˜35 nucleotides upstream and ˜40 nucleotides downstream) arise from restriction enzyme sites within the multiple cloning site of the siRNA. As used herein the term “miRNA” encompasses both the naturally occurring miRNA sequences as well as artificially generated miRNA shuttle vectors.

The siRNA can be encoded by a nucleic acid sequence, and the nucleic acid sequence can also include a promoter. The nucleic acid sequence can also include a polyadenylation signal. In some embodiments, the polyadenylation signal is a synthetic minimal polyadenylation signal or a sequence of six Ts.

“Off-target toxicity” refers to deleterious, undesirable, or unintended phenotypic changes of a host cell that expresses or contains a siRNA. Off-target toxicity may result in loss of desirable function, gain of non-desirable function, or even death at the cellular or organismal level. Off-target toxicity may occur immediately upon expression of the siRNA or may occur gradually over time. Off-target toxicity may occur as a direct result of the expression siRNA or may occur as a result of induction of host immune response to the cell expressing the siRNA. Without wishing to be bound by theory, off-target toxicity is postulated to arise from high levels or overabundance of RNAi substrates within the cell. These overabundant or overexpressed RNAi substrates, including without limitation pre- or pri RNAi substrates as well as overabundant mature antisense-RNAs, may compete for endogenous RNAi machinery, thus disrupting natural miRNA biogenesis and function. Off-target toxicity may also arise from an increased likelihood of silencing of unintended mRNAs (i.e., off-target) due to partial complementarity of the sequence. Off target toxicity may also occur from improper strand biasing of a non-guide region such that there is preferential loading of the non-guide region over the targeted or guide region of the RNAi. Off-target toxicity may also arise from stimulation of cellular responses to dsRNAs which include dsRNA. “Decreased off target toxicity” refers to a decrease, reduction, abrogation or attenuation in off target toxicity such that the therapeutic effect is more beneficial to the host than the toxicity is limiting or detrimental as measured by an improved duration or quality of life or an improved sign or symptom of a disease or condition being targeted by the siRNA. “Limited off target toxicity” or “low off target toxicity” refer to unintended undesirable phenotypic changes to a cell or organism, whether detectable or not, that does not preclude or outweigh or limit the therapeutic benefit to the host treated with the siRNA and may be considered a “side effect” of the therapy. Decreased or limited off target toxicity may be determined or inferred by comparing the in vitro analysis such as Northern blot or qPCR for the levels of siRNA substrates or the in vivo effects comparing an equivalent shRNA vector to the miRNA shuttle vector of the present invention.

“Knock-down,” “knock-down technology” refers to a technique of gene silencing in which the expression of a target gene is reduced as compared to the gene expression prior to the introduction of the siRNA, which can lead to the inhibition of production of the target gene product. The term “reduced” is used herein to indicate that the target gene expression is lowered by 1-100%. In other words, the amount of RNA available for translation into a polypeptide or protein is minimized. For example, the amount of protein may be reduced by 10, 20, 30, 40, 50, 60, 70, 80, 90, 95, or 99%. In some embodiments, the expression is reduced by about 90% (i.e., only about 10% of the amount of protein is observed a cell as compared to a cell where siRNA molecules have not been administered). Knock-down of gene expression can be directed by the use of RNAi molecules.

According to a method of the present invention, the expression of CF is modified via RNAi. For example, SIN3A expression and/or function is suppressed in a cell. The term “suppressing” refers to the diminution, reduction or elimination in the number or amount of transcripts present in a particular cell. It also relates to reductions in functional protein levels by inhibition of protein translation, which do not necessarily correlate with reductions in mRNA levels. For example, the accumulation of mRNA encoding SIN3A is suppressed in a cell by RNA interference (RNAi), e.g., the gene is silenced by sequence-specific double-stranded RNA (dsRNA), which is also called small interfering RNA (siRNA). These siRNAs can be two separate RNA molecules that have hybridized together, or they may be a single hairpin wherein two portions of a RNA molecule have hybridized together to form a duplex.

A mutant protein refers to the protein encoded by a gene having a mutation, e.g., a missense or nonsense mutation in one or both alleles of a gene, such as CFTR, causing disease. The term “gene” is used broadly to refer to any segment of nucleic acid associated with a biological function. Thus, genes include coding sequences and/or the regulatory sequences required for their expression. For example, “gene” refers to a nucleic acid fragment that expresses mRNA, functional RNA, or specific protein, including regulatory sequences. “Genes” also include nonexpressed DNA segments that, for example, form recognition sequences for other proteins. “Genes” can be obtained from a variety of sources, including cloning from a source of interest or synthesizing from known or predicted sequence information, and may include sequences designed to have desired parameters. An “allele” is one of several alternative forms of a gene occupying a given locus on a chromosome.

The term “nucleic acid” refers to deoxyribonucleic acid (DNA) or ribonucleic acid (RNA) and polymers thereof in either single- or double-stranded form, composed of monomers (nucleotides) containing a sugar, phosphate and a base that is either a purine or pyrimidine. Unless specifically limited, the term encompasses nucleic acids containing known analogs of natural nucleotides that have similar binding properties as the reference nucleic acid and are metabolized in a manner similar to naturally occurring nucleotides. Unless otherwise indicated, a particular nucleic acid sequence also encompasses conservatively modified variants thereof (e.g., degenerate codon substitutions) and complementary sequences, as well as the sequence explicitly indicated. Specifically, degenerate codon substitutions may be achieved by generating sequences in which the third position of one or more selected (or all) codons is substituted with mixed-base and/or deoxyinosine residues. A “nucleic acid fragment” is a portion of a given nucleic acid molecule.

A “nucleotide sequence” is a polymer of DNA or RNA that can be single-stranded or double-stranded, optionally containing synthetic, non-natural or altered nucleotide bases capable of incorporation into DNA or RNA polymers.

The terms “nucleic acid,” “nucleic acid molecule,” “nucleic acid fragment,” “nucleic acid sequence or segment,” or “polynucleotide” are used interchangeably and may also be used interchangeably with gene, cDNA, DNA and RNA encoded by a gene.

The invention encompasses isolated or substantially purified nucleic acid nucleic acid molecules and compositions containing those molecules. In the context of the present invention, an “isolated” or “purified” DNA molecule or RNA molecule is a DNA molecule or RNA molecule that exists apart from its native environment and is therefore not a product of nature. An isolated DNA molecule or RNA molecule may exist in a purified form or may exist in a non-native environment such as, for example, a transgenic host cell. For example, an “isolated” or “purified” nucleic acid molecule or biologically active portion thereof, is substantially free of other cellular material, or culture medium when produced by recombinant techniques, or substantially free of chemical precursors or other chemicals when chemically synthesized. In one embodiment, an “isolated” nucleic acid is free of sequences that naturally flank the nucleic acid (i.e., sequences located at the 5′ and 3′ ends of the nucleic acid) in the genomic DNA of the organism from which the nucleic acid is derived. For example, in various embodiments, the isolated nucleic acid molecule can contain less than about 5 kb, 4 kb, 3 kb, 2 kb, 1 kb, 0.5 kb, or 0.1 kb of nucleotide sequences that naturally flank the nucleic acid molecule in genomic DNA of the cell from which the nucleic acid is derived. Fragments and variants of the disclosed nucleotide sequences are also encompassed by the present invention. By “fragment” or “portion” is meant a full length or less than full length of the nucleotide sequence.

“Naturally occurring,” “native,” or “wild-type” is used to describe an object that can be found in nature as distinct from being artificially produced. For example, a protein or nucleotide sequence present in an organism (including a virus), which can be isolated from a source in nature and that has not been intentionally modified by a person in the laboratory, is naturally occurring.

A “variant” of a molecule is a sequence that is substantially similar to the sequence of the native molecule. For nucleotide sequences, variants include those sequences that, because of the degeneracy of the genetic code, encode the identical amino acid sequence of the native protein. Naturally occurring allelic variants such as these can be identified with the use of molecular biology techniques, as, for example, with polymerase chain reaction (PCR) and hybridization techniques. Variant nucleotide sequences also include synthetically derived nucleotide sequences, such as those generated, for example, by using site-directed mutagenesis, which encode the native protein, as well as those that encode a polypeptide having amino acid substitutions. Generally, nucleotide sequence variants of the invention will have at least 40%, 50%, 60%, to 70%, e.g., 71%, 72%, 73%, 74%, 75%, 76%, 77%, 78%, to 79%, generally at least 80%, e.g., 81%-84%, at least 85%, e.g., 86%, 87%, 88%, 89%, 90%, 91%, 92%, 93%, 94%, 95%, 96%, 97%, to 98%, sequence identity to the native (endogenous) nucleotide sequence.

The terms “protein,” “peptide” and “polypeptide” are used interchangeably herein.

“Functional RNA” refers to sense RNA, antisense RNA, ribozyme RNA, siRNA, or other RNA that may not be translated but yet has an effect on at least one cellular process.

The term “RNA transcript” or “transcript” refers to the product resulting from RNA polymerase catalyzed transcription of a DNA sequence. When the RNA transcript is a perfect complementary copy of the DNA sequence, it is referred to as the primary transcript or it may be a RNA sequence derived from posttranscriptional processing of the primary transcript and is referred to as the mature RNA. “Messenger RNA” (mRNA) refers to the RNA that is without introns and that can be translated into protein by the cell.

“Operably-linked” refers to the association of nucleic acid sequences on single nucleic acid fragment so that the function of one of the sequences is affected by another. For example, a regulatory DNA sequence is said to be “operably linked to” or “associated with” a DNA sequence that codes for an RNA or a polypeptide if the two sequences are situated such that the regulatory DNA sequence affects expression of the coding DNA sequence (i.e., that the coding sequence or functional RNA is under the transcriptional control of the promoter). Coding sequences can be operably-linked to regulatory sequences in sense or antisense orientation.

“Expression” refers to the transcription and/or translation of an endogenous gene, heterologous gene or nucleic acid segment, or a transgene in cells. For example, in the case of siRNA constructs, expression may refer to the transcription of the siRNA only. In addition, expression refers to the transcription and stable accumulation of sense (mRNA) or functional RNA. Expression may also refer to the production of protein.

The siRNAs of the present invention can be generated by any method known to the art, for example, by in vitro transcription, recombinantly, or by synthetic means. In one example, the siRNAs can be generated in vitro by using a recombinant enzyme, such as T7 RNA polymerase, and DNA oligonucleotide templates.

### Administration of Therapeutic Agent

The therapeutic agent is administered to the patient so that the therapeutic agent contacts cells of the patient's respiratory or digestive system. For example, the therapeutic agent may be administered directly via an airway to cells of the patient's respiratory system. The therapeutic agent can be administered intranasally (e.g., nose drops) or by inhalation via the respiratory system, such as by propellant based metered dose inhalers or dry powders inhalation devices.

Formulations suitable for administration include liquid solutions. Liquid formulations may include diluents, such as water and alcohols, for example, ethanol, benzyl alcohol, propylene glycol, glycerin, and the polyethylene alcohols, either with or without the addition of a pharmaceutically acceptable surfactant, suspending agent, or emulsifying agent. The therapeutic agent can be administered in a physiologically acceptable diluent in a pharmaceutically acceptable carrier, such as a sterile liquid or mixture of liquids, including water, saline, aqueous dextrose and related sugar solutions, an alcohol, such as ethanol, isopropanol, or hexadecyl alcohol, glycols, such as propylene glycol or polyethylene glycol such as poly(ethyleneglycol) 400, glycerol ketals, such as 2,2-dimethyl-1,3-dioxolane-4-methanol, ethers, an oil, a fatty acid, a fatty acid ester or glyceride, or an acetylated fatty acid glyceride with or without the addition of a pharmaceutically acceptable surfactant, such as a soap or a detergent, suspending agent, such as pectin, carbomers, methylcellulose, hydroxypropylmethylcellulose, or carboxymethylcellulose, or emulsifying agents and other pharmaceutical adjuvants.

The therapeutic agent, alone or in combination with other suitable components, can be made into aerosol formulations to be administered via inhalation. These aerosol formulations can be placed into pressurized acceptable propellants, such as dichlorodifluoromethane, propane, and nitrogen. Such aerosol formulations may be administered by metered dose inhalers. They also may be formulated as pharmaceuticals for non-pressured preparations, such as in a nebulizer or an atomizer. In certain embodiments, administration may be, e.g., aerosol, instillation, intratracheal, intrabronchial or bronchoscopic deposition.

In certain embodiments, the therapeutic agent may be administered in a pharmaceutical composition. Such pharmaceutical compositions may also comprise a pharmaceutically acceptable carrier and other ingredients known in the art. The pharmaceutically acceptable carriers described herein, including, but not limited to, vehicles, adjuvants, excipients, or diluents, are well-known to those who are skilled in the art. Typically, the pharmaceutically acceptable carrier is chemically inert to the active compounds and has no detrimental side effects or toxicity under the conditions of use. The pharmaceutically acceptable carriers can include polymers and polymer matrices. Viscoelastic gel formulations with, e.g., methylcellulose and/or carboxymethylcellulose may be beneficial (see Sinn et al., Am J Respir Cell Mol Biol, 32(5), 404-410 (2005)).

The therapeutic agent can be administered by any conventional method available for use in conjunction with pharmaceuticals, either as individual therapeutic agents or in combination with at least one additional therapeutic agent.

In certain embodiments, the therapeutic agent are administered with an agent that disrupts, e.g., transiently disrupts, tight junctions, such as EGTA (see U.S. Pat. No. 6,855,549).

The total amount of the therapeutic agent administered will also be determined by the route, timing and frequency of administration as well as the existence, nature, and extent of any adverse side effects that might accompany the administration of the compound and the desired physiological effect. It will be appreciated by one skilled in the art that various conditions or disease states, in particular chronic conditions or disease states, may require prolonged treatment involving multiple administrations.

The therapeutic agent can be formulated as pharmaceutical compositions and administered to a mammalian host, such as a human patient in a variety of forms adapted to the chosen route of administration, i.e., orally or parenterally, by intravenous, intramuscular, topical or subcutaneous routes.

Thus, the present compounds may be systemically administered, e.g., orally, in combination with a pharmaceutically acceptable vehicle such as an inert diluent or an assimilable edible carrier. They may be enclosed in hard or soft shell gelatin capsules, may be compressed into tablets, or may be incorporated directly with the food of the patient's diet. For oral therapeutic administration, the active compound may be combined with one or more excipients and used in the form of ingestible tablets, buccal tablets, troches, capsules, elixirs, suspensions, syrups, wafers, and the like. Such compositions and preparations should contain at least 0.1% of active compound. The percentage of the compositions and preparations may, of course, be varied and may conveniently be between about 2 to about 60% of the weight of a given unit dosage form. The amount of active compound in such therapeutically useful compositions is such that an effective dosage level will be obtained.

The tablets, troches, pills, capsules, and the like may also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the active compound may be incorporated into sustained-release preparations and devices.

The therapeutic agent may also be administered intravenously or intraperitoneally by infusion or injection. Solutions of the active compound or its salts can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

The pharmaceutical dosage forms suitable for injection or infusion can include sterile aqueous solutions or dispersions or sterile powders comprising the active ingredient which are adapted for the extemporaneous preparation of sterile injectable or infusible solutions or dispersions, optionally encapsulated in liposomes. In all cases, the ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions are prepared by incorporating the active compound in the required amount in the appropriate solvent with various of the other ingredients enumerated above, as required, followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions.

For topical administration, the present compounds may be applied in pure form, i.e., when they are liquids. However, it will generally be desirable to administer them to the skin as compositions or formulations, in combination with a dermatologically acceptable carrier, which may be a solid or a liquid.

Useful solid carriers include finely divided solids such as talc, clay, microcrystalline cellulose, silica, alumina and the like. Useful liquid carriers include water, alcohols or glycols or water-alcohol/glycol blends, in which the present compounds can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

Examples of useful dermatological compositions which can be used to deliver the compounds of formula I to the skin are known to the art; for example, see Jacquet et al. (U.S. Pat. No. 4,608,392), Geria (U.S. Pat. No. 4,992,478), Smith et al. (U.S. Pat. No. 4,559,157) and Wortzman (U.S. Pat. No. 4,820,508).

Useful dosages of the therapeutic agent can be determined by comparing their in vitro activity, and in vivo activity in animal models. Methods for the extrapolation of effective dosages in mice, and other animals, to humans are known to the art; for example, see U.S. Pat. No. 4,938,949.

The amount of the therapeutic agent, or an active salt or derivative thereof, required for use in treatment will vary not only with the particular salt selected but also with the route of administration, the nature of the condition being treated and the age and condition of the patient and will be ultimately at the discretion of the attendant physician or clinician.

Pharmaceutical compositions are administered in an amount, and with a frequency, that is effective to inhibit or alleviate the symptoms of cystic fibrosis and/or to delay the progression of the disease. The effect of a treatment may be clinically determined by nasal potential difference measurements as described herein. The precise dosage and duration of treatment may be determined empirically using known testing protocols or by testing the compositions in model systems known in the art and extrapolating therefrom. Dosages may also vary with the severity of the disease. A pharmaceutical composition is generally formulated and administered to exert a therapeutically useful effect while minimizing undesirable side effects. In general, an oral dose ranges from about 200 mg to about 1000 mg, which may be administered 1 to 3 times per day. Compositions administered as an aerosol are generally designed to provide a final concentration of about 10 to 50 μM at the airway surface, and may be administered 1 to 3 times per day. It will be apparent that, for any particular subject, specific dosage regimens may be adjusted over time according to the individual need. In general, however, a suitable dose will be in the range of from about 0.5 to about 100 mg/kg, e.g., from about 10 to about 75 mg/kg of body weight per day, such as 3 to about 50 mg per kilogram body weight of the recipient per day, preferably in the range of 6 to 90 mg/kg/day, most preferably in the range of 15 to 60 mg/kg/day.

The compound is conveniently formulated in unit dosage form; for example, containing 5 to 1000 mg, conveniently 10 to 750 mg, most conveniently, 50 to 500 mg of active ingredient per unit dosage form. In one embodiment, the invention provides a composition comprising a compound of the invention formulated in such a unit dosage form.

The desired dose may conveniently be presented in a single dose or as divided doses administered at appropriate intervals, for example, as two, three, four or more sub-doses per day. The sub-dose itself may be further divided, e.g., into a number of discrete loosely spaced administrations; such as multiple inhalations from an insufflator or by application of a plurality of drops into the eye.

Compounds of the invention can also be administered in combination with other therapeutic agents, for example, other agents that are useful to treat cystic fibrosis. Examples of such agents include antibiotics. Accordingly, in one embodiment the invention also provides a composition comprising a therapeutic agent, or a pharmaceutically acceptable salt thereof, at least one other therapeutic agent, and a pharmaceutically acceptable diluent or carrier. The invention also provides a kit comprising a therapeutic agent, or a pharmaceutically acceptable salt thereof, at least one other therapeutic agent, packaging material, and instructions for administering the therapeutic agent or the pharmaceutically acceptable salt thereof and the other therapeutic agent or agents to an animal to treat cystic fibrosis.

A pharmaceutical composition may be prepared with carriers that protect active ingredients against rapid elimination from the body, such as time release formulations or coatings. Such carriers include controlled release formulations, such as, but not limited to, microencapsulated delivery systems, and biodegradable, biocompatible polymers, such as ethylene vinyl acetate, polyanhydrides, polyglycolic acid, polyorthoesters, polylactic acid and others known to those of ordinary skill in the art.

In certain embodiments, the therapeutic agent is directly administered as a pressurized aerosol or nebulized formulation to the patient's lungs via inhalation. Such formulations may contain any of a variety of known aerosol propellants useful for endopulmonary and/or intranasal inhalation administration. In addition, water may be present, with or without any of a variety of cosolvents, surfactants, stabilizers (e.g., antioxidants, chelating agents, inert gases and buffers). For compositions to be administered from multiple dose containers, antimicrobial agents are typically added. Such compositions are also generally filtered and sterilized, and may be lyophilized to provide enhanced stability and to improve solubility.

As noted above, a therapeutic agent may be administered to a mammal to stimulate chloride transport, and to treat cystic fibrosis. Patients that may benefit from administration of a therapeutic compound as described herein are those afflicted with cystic fibrosis. Such patients may be identified based on standard criteria that are well known in the art, including the presence of abnormally high salt concentrations in the sweat test, the presence of high nasal potentials, or the presence of a cystic fibrosis-associated mutation. Activation of chloride transport may also be beneficial in other diseases that show abnormally high mucus accumulation in the airways, such as asthma and chronic bronchitis. Similarly, intestinal constipation may benefit from activation of chloride transport by the therapeutic agents provided herein.

The term “therapeutically effective amount,” in reference to treating a disease state/condition, refers to an amount of a compound either alone or as contained in a pharmaceutical composition that is capable of having any detectable, positive effect on any symptom, aspect, or characteristics of a disease state/condition when administered as a single dose or in multiple doses. Such effect need not be absolute to be beneficial.

The terms “treat,” “treating” and “treatment” as used herein include administering a compound prior to the onset of clinical symptoms of a disease state/condition so as to prevent any symptom, as well as administering a compound after the onset of clinical symptoms of a disease state/condition so as to reduce or eliminate any symptom, aspect or characteristic of the disease state/condition. Such treating need not be absolute to be useful.

### Example 1

### A Novel microRNA Network Regulates Expression and Biosynthesis of CFTR and CFTR-ΔF508

Production of functional proteins requires multiple steps including gene transcription and post-translational processing. MicroRNAs (miRNA) can regulate individual stages of these processes. Despite the importance of the cystic fibrosis transmembrane conductance regulator (CFTR) channel for epithelial anion transport, how its expression is regulated remains uncertain. Here we show that microRNA-138 regulates CFTR expression through its interactions with the transcriptional regulatory protein SIN3A. Treating airway epithelia with a miR-138 mimic increased CFTR mRNA. Surprisingly, miR-138 also enhanced CFTR abundance and transepithelial Cl⁻permeability independently of elevated mRNA levels. A miR-138 anti-miR had the opposite effects. Importantly, miR-138 altered the expression of many genes encoding proteins that associate with CFTR and may influence its biosynthesis. The most common CFTR mutation, ΔF508, causes protein misfolding, degradation, and cystic fibrosis (CF). Remarkably, manipulating the miR-138 regulatory network also improved biosynthesis of CFTR-ΔF508 and restored Cl⁻ transport to CF airway epithelia. This novel miRNA-regulated network directs gene expression from the chromosome to the cell membrane, indicating that an individual miRNA can control a cellular process broader than previously recognized. This discovery also provides a new target for restoring CFTR function to cells affected by the most common CF mutation.

Mutations in CFTR cause CF, an autosomal recessive disease characterized by progressive pulmonary infection and inflammation. CFTR is a low abundance mRNA in airway epithelia and its temporal and spatial expression are tightly regulated. Though the CFTR promoter has been extensively studied, its complex regulation remains unexplained. Because microRNAs (miRNA) play key roles in the transcriptional and post-transcriptional regulation of 60% or more of human genes, they may provide a previously unidentified mechanism for regulating CFTR abundance. We profiled global miRNA expression in well-differentiated primary cultures of human airway epithelia by quantitative PCR. Of 115 identified miRNAs, 31 were highly expressed (C_{q}<25) (Table 1). Targetscan, Pictar, and Miranda software-based analyses of these 31 miRNAs identified the SIN3A (SINS homolog A) gene as a conserved candidate miR-138 target. SIN3A is a transcriptional regulator belonging to the Sin3/HDAC (histone deacetylase) core complex. Notably, SIN3A protein has conserved motifs that bind to the chromatin insulator protein CCCTC-binding factor (CTCF), a ubiquitously expressed, highly conserved transcriptional repressor that recruits SIN3A and other proteins to the promoters of target genes. DNA methylation of the CFTR promoter across cell lines correlates inversely with transcription, suggesting that CFTR is transcriptionally regulated. Importantly, the CFTR locus contains functional CTCF binding sites. We thus hypothesized that miR-138 and SIN3A regulate CFTR.

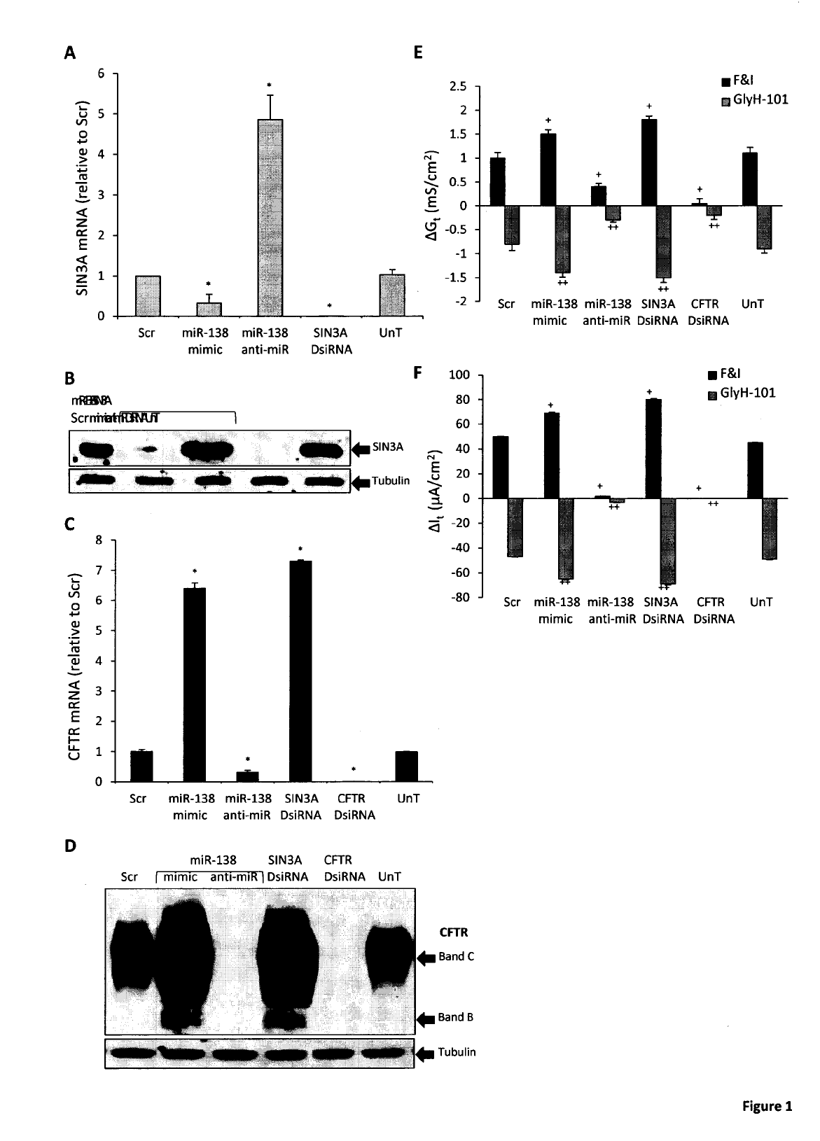

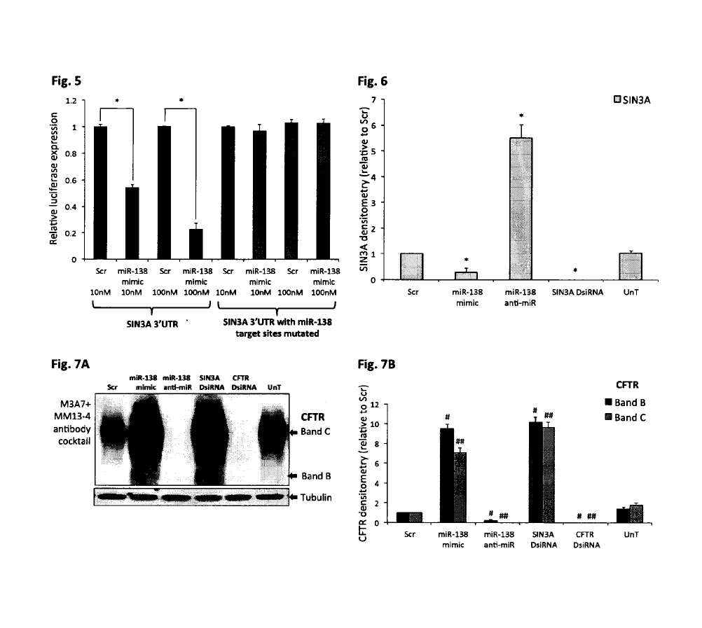

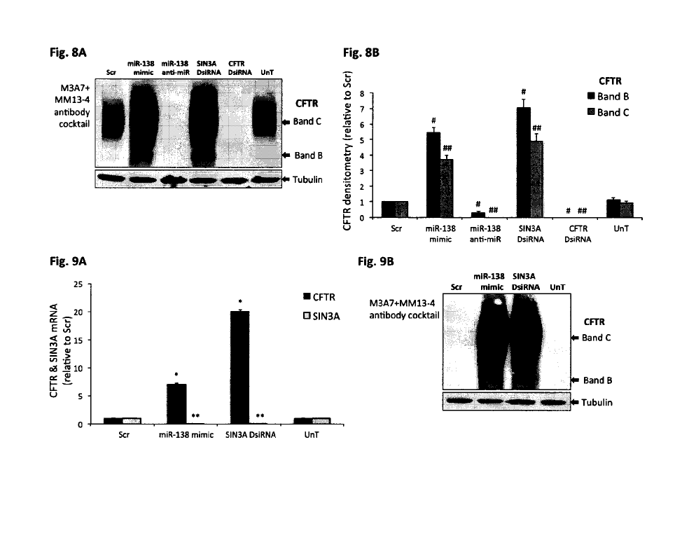

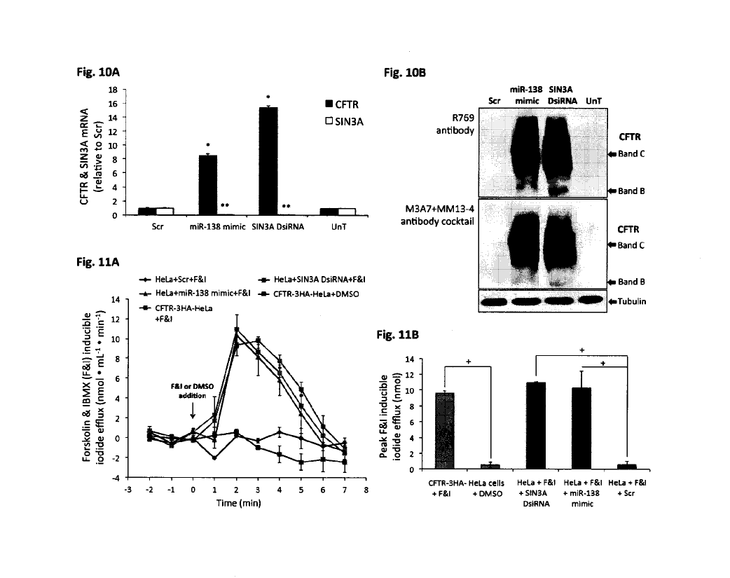

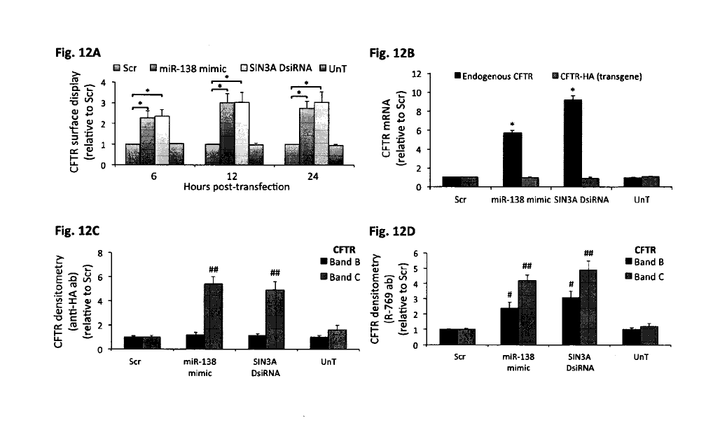

A dual-luciferase reporter assay revealed that miR-138 repressed SIN3A expression in a dose-dependent manner, by binding to its 3′UTR (FIG. 5). This effect was site-specific; mutating the two miR-138 binding sites in the SIN3A 3′UTR relieved the repression in vitro. Transfection of polarized primary cultures of human airway epithelia with a miR-138 mimic reduced, and that of a miR-138 anti-miR increased, SIN3A mRNA and protein levels (FIG. 1a, b, FIG. 6). These findings validate SIN3A as a miR-138 target in airway epithelia.

To test the hypothesis that miR-138 regulates SIN3A and thereby CFTR expression in airway epithelia we used the Calu-3 cell line, which expresses CFTR. Treatment of Calu-3 cells with a miR-138 mimic or a Dicer-substrate siRNA (DsiRNA) against SIN3A increased CFTR mRNA and protein levels (FIG. 1c, d, FIG. 7), while the miR-138 anti-miR markedly reduced CFTR mRNA and protein abundance (FIG. 1c, d, FIG. 7). CFTR creates an ion permeability and therefore its function can be assessed by measuring transepithelial electrical conductance. The miR-138 mimic and SIN3A DsiRNA treatments increased CFTR-mediated conductance (Gₜ) and current (Iₜ) in polarized Calu-3 epithelia, while the miR-138 anti-miR had the opposite effects (FIG. 1e, f).

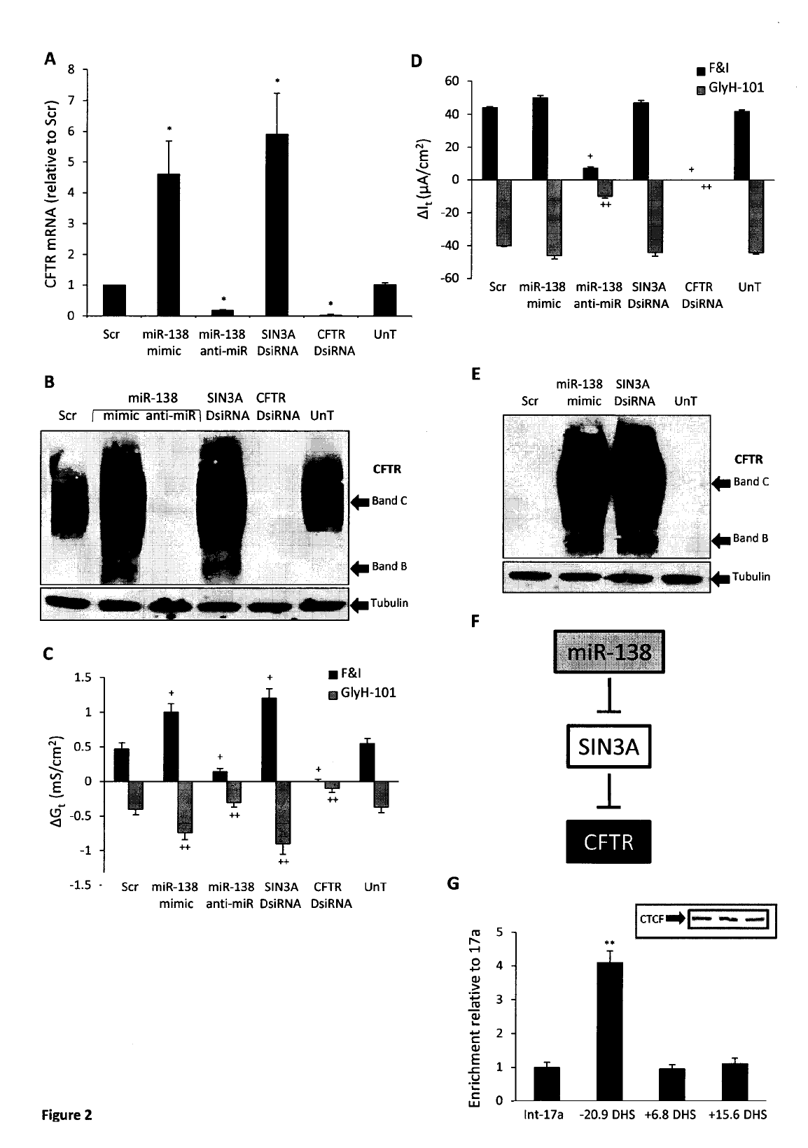

In polarized primary cultures of human airway epithelia, transfection with a miR-138 mimic or SIN3A DsiRNA increased, and that of a miR-138 anti-miR reduced, CFTR mRNA and protein levels (FIG. 2a, b, FIG. 8). Treatment with the miR-138 mimic and the SIN3A DsiRNA increased cAMP-stimulated Gₜ (FIG. 2c). There was no change in Iₜ (FIG. 2d), consistent with the presence of other rate-limiting steps for Cl⁻ secretion in airway epithelia (Farmen, S. L. et al. Gene transfer of CFTR to airway epithelia: low levels of expression are sufficient to correct Cl⁻ transport and overexpression can generate basolateral CFTR. Am. J Physiol. Lung Cell Mol. Physiol. 289, L1123-1130 (2005)). The miR-138 anti-miR reduced both Gᵣ and Iₜ responses to cAMP-dependent stimulation (FIG. 2c, d).

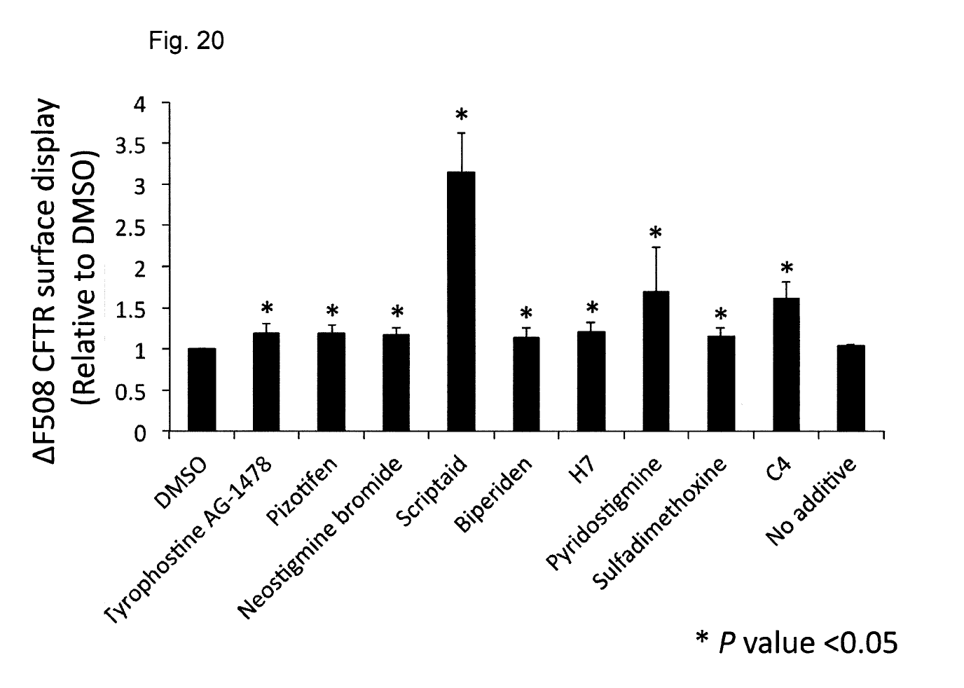

These data show that miR-138 and SIN3A regulate CFTR expression in epithelia that normally express CFTR. To learn whether they can also control CFTR expression in cells that do not produced CFTR, we studied HeLa and HEK293T cells. The miR-138 mimic and the SIN3A DsiRNA markedly increased CFTR mRNA and protein expression (FIG. 2e, FIG. 9, 10). Transfected HeLa cells also exhibited a cAMP-dependent anion permeability, as assessed by iodide efflux (FIG. 11). These results implicate SIN3A as a potent regulator of CFTR expression, and further support the notion that miR-138 regulates CFTR expression by repressing SIN3A (FIG. 20.

To assess whether SIN3A-mediated CFTR repression involves CTCF-mediated recruitment of SIN3A to the CFTR promoter (Ellison-Zelski, S. J., Solodin, N. M. & Alarid, E. T. Repression of ESR1 through actions of estrogen receptor alpha and Sin3A at the proximal promoter. Mol. Cell Biol. 29, 4949-4958 (2009)), we performed chromatin immunoprecipitation in primary cultures of non-CF human airway epithelia. Specifically, we assessed SIN3A enrichment at two known CTCF binding sites within DNase I hypersensitive sites (DHS) of the CFTR locus: −20.9 DHS (distance from transcriptional start site) and +6.8 DHS (distance from transcriptional stop site) (Blackledge, N. P. et al. CTCF mediates insulator function at the CFTR locus. Biochem. J 408, 267-275 (2007); Blackledge, N. P., Ott, C. J., Gillen, A. E. & Harris, A. An insulator element 3′ to the CFTR gene binds CTCF and reveals an active chromatin hub in primary cells. Nucleic Acids Res. 37, 1086-1094 (2009)). Indeed, the -20.9 DHS was enriched for SIN3A compared to two control regions (CFTR intron 17a and +15.6 kb DHS) (FIG. 2g).

To learn whether miR-138 and SIN3A might have post-transcriptional effects on protein biosynthesis in addition to their direct transcriptional regulation of CFTR, we performed additional experiments using HeLa cells stably expressing HA-tagged wild-type CFTR under control of the CMV promoter. A cell-based ELISA using an HA-antibody revealed an increase of HA-tagged CFTR at the cell surface following treatment with the miR-138 mimic or SIN3A DsiRNA (FIG. 3a, FIG. 12a), without changes in transgene mRNA abundance (FIG. 12b). This result was further supported by immunoblots (FIG. 3b, FIG. 12c, d). These data indicate that miR-138 has important post-transcriptional effects on CFTR biosynthesis.

Subsequent global mRNA transcript profiling in Calu-3 epithelia treated with the miR-138 mimic or SIN3A DsiRNA identified a common set of 773 genes whose expression changed in response to these interventions (FIG. 3c). On intersecting these gene sets with a curated list of 362 genes with protein products known to associate with CFTR (CFTR-Associated Gene Network, Table 2), 34.5% (125/362) were in the CFTR-Associated Gene Network, a significant enrichment over random expectations (FIG. 3c, Table 3). These 125 genes function in several cellular compartments and many positively influence CFTR protein expression or stability (Table 4). These findings further support the conclusion that miR-138 enhances CFTR biogenesis.

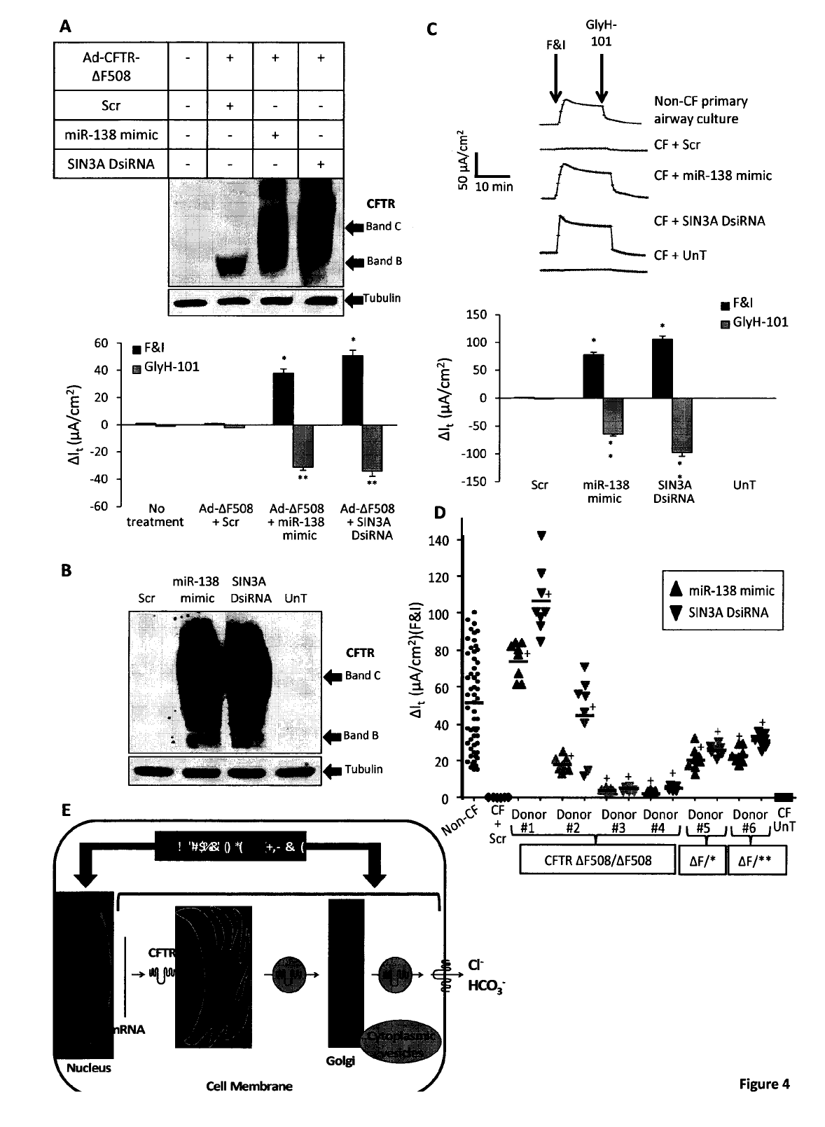

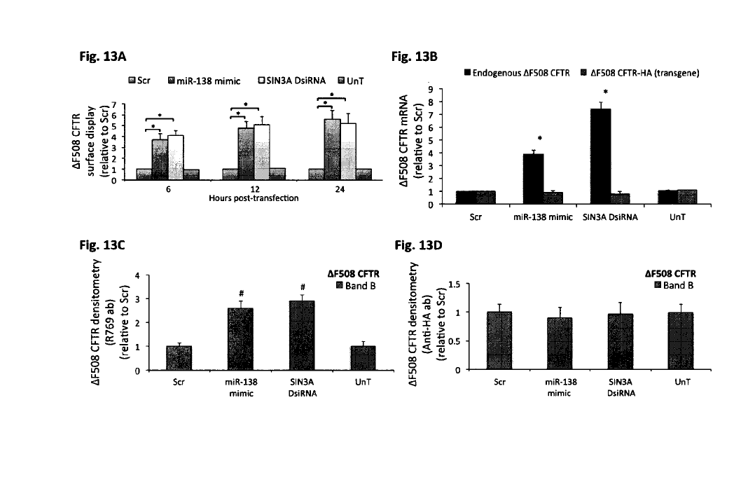

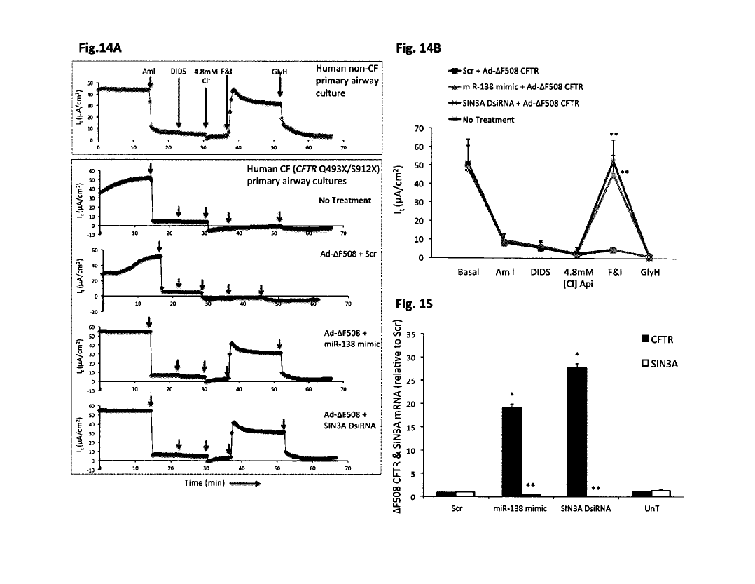

The most common CFTR mutant, ΔF508, generates a protein with an altered structure that is unstable, mislocalized, and rapidly degraded via ER-associated degradation. Interventions that improve biosynthetic processing, such as low temperature, chemical chaperones, and small molecules, can partially restore CFTR□ΔF508 anion channel function. However, overexpression of the ΔF508 cDNA in heterologous cells or primary airway epithelia does not restore CFTR-dependent anion conductance. Because miR-138 increased the biosynthesis of wild-type CFTR (FIG. 3a, b), we hypothesized that it might also improve the biosynthesis of CFTR-ΔF508. HeLa cells stably expressing HA-tagged CFTR-ΔF508 cDNA under the control of the CMV promoter (Okiyoneda, T. et al. Peripheral protein quality control removes unfolded CFTR from the plasma membrane. Science 329, 805-810 (2010)) were transfected with the miR-138 mimic or SIN3A DsiRNA. Surprisingly, we found that mutant CFTR reached the cell surface (ELISA, FIG. 3d, FIG. 13a), without a change in transgene mRNA abundance (FIG. 13b). Immunoblotting with an HA-antibody detecting only the transgene protein product demonstrated that both interventions increased the abundance of the mature, fully glycosylated CFTR band C (FIG. 3e, FIG. 13c, d). We also expressed a recombinant CMV promoter-driven CFTR-ΔF508 cDNA in primary human CFTR null airway epithelia (CFTR Q493X/S912X) using an adenovirus (Ad) vector (Ostedgaard, L. S. et al. Processing and function of CFTR-DeltaF508 are species-dependent. Proc. Natl. Acad. Sci. USA 104, 15370-15375 (2007)). In this setting CFTR-mediated Cl⁻ current was restored only in epithelia pretreated with the miR-138 mimic or SIN3A DsiRNA (FIG. 4a, FIG. 14). These results further indicate that miR-138 and SIN3A regulated genes influence the post-transcriptional processing of CFTR.

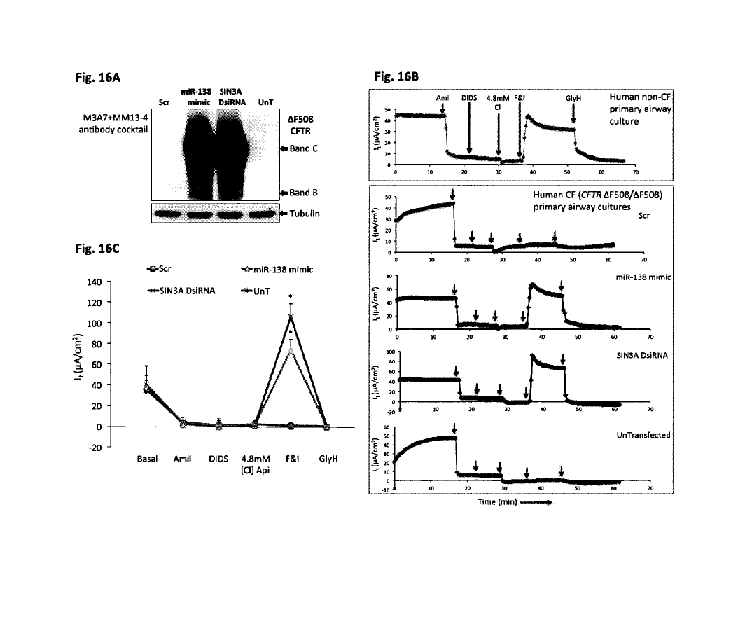

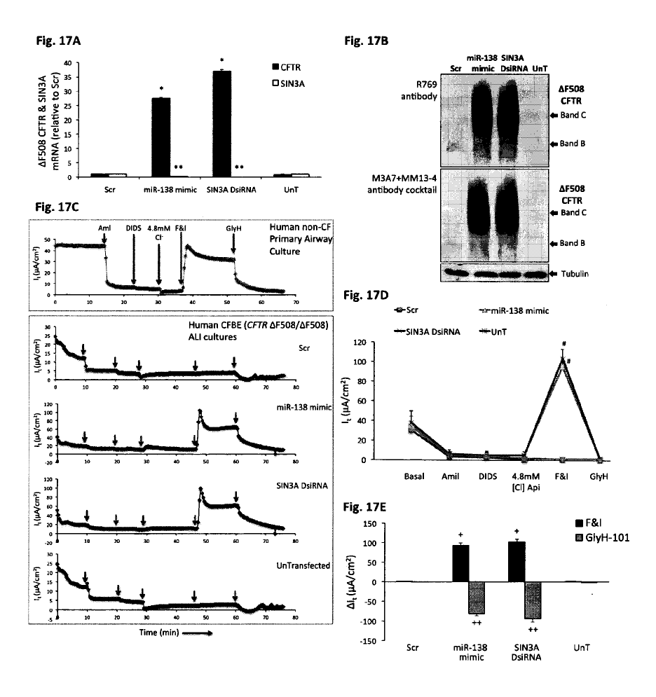

Expressing the miR-138 mimic or SIN3A DsiRNA increased CFTR-ΔF508 mRNA and protein even in primary cultures of CF airway epithelia (FIG. 4b, FIG. 15, 16). Remarkably, they also restored CFTR-mediated Cl⁻ transport in these epithelia (FIG. 4c, FIG. 16b, c). Restoration of CFTR-mediated Cl⁻ transport was observed in primary CF epithelia from multiple human donors (FIG. 4d), and similar results were obtained in a cell line homozygous for the ΔF508 mutation (FIG. 17).

Here we show that miR-138, acting via SIN3A and other target genes, is a key regulator of CFTR, at both the levels of mRNA transcription and protein biosynthesis (FIG. 4e, Tables 3, 4). MiR-138 orchestrates a cellular program that influences wild-type and mutant CFTR similarly, increasing the biogenesis and cell-surface delivery of both. The previously unknown miR-138/SIN3A regulated gene network represents a new therapeutic target for rescuing CFTR ΔF508 function. These discoveries also raise the possibility that manipulating miR-138/SIN3A and their targets might restore function of misprocessed proteins associated with other genetic diseases.

METHODS

Primary Human Airway Epithelia:

Airway epithelia from human trachea and primary bronchus removed from organs donated for research were cultured at the air-liquid interface (ALI) (Karp, P. H. et al. An in vitro model of differentiated human airway epithelia. Methods for establishing primary cultures. Methods Mol. Biol. 188, 115-137 (2002)). These studies were approved by the Institutional Review Board of the University of Iowa. Briefly, airway epithelial cells were dissociated from native tissue by pronase enzyme digestion. Permeable membrane inserts (0.6 cm² Millipore-PCF, 0.33 cm² Costar-Polyester) pre-coated with human placental collagen (IV, Sigma) were seeded with freshly dissociated epithelia. Seeding culture media used was DMEM/F-12 medium supplemented with 5% FBS, 50 units/mL penicillin, 50 μg/mL streptomycin, 50 μg/mL gentamicin, 2 μg/mL fluconazole, and 1.25 μg/mL amphotericin B. For epithelia from cystic fibrosis (CF) patients, the following additional antibiotics were used for the first 5 days: 77 μg/mL ceftazidime, 12.5 μg/mL imipenem and cilastatin, 80 μg/mL tobramycin, 25 μg/mL piperacillin and tazobactam, 20 μg/mL sulfamethoxazole, and 4 μg/mL trimethoprim. After seeding, the cultures were maintained in DMEM/F-12 medium supplemented with 2% Ultroser G (USG, Pall Biosepra) and the above listed antibiotics.

RNA Isolation:

Total RNA from human primary airway epithelial cultures, and cell lines (Calu-3, HEK293T, HeLa, CFBE) was isolated using the mirVana™ miRNA isolation kit (Ambion) (Ramachandran, S., Clarke, L. A., Scheetz, T. E., Amaral, M. D. & McCray, P. B., Jr. Microarray mRNA expression profiling to study cystic fibrosis. Methods Mol. Biol. 742, 193-212 (2011)). Total RNA was tested on an Agilent Model 2100 Bioanalyzer (Agilent Technologies). Only samples with an RNA integrity number (RIN) over 7.0 were selected for downstream processing.

TaqMan Low Density microRNA Array (TLDA):

Global microRNA (miRNA) expression profiling was performed using the TaqMan® Human MicroRNA Array Set v2.0 (Applied Biosystems), which screens for the expression of 667 human miRNAs plus endogenous controls. Total RNA was isolated from primary cultures (a minimum of 30 days post-seeding) from 4 human non-CF donors, reverse transcribed using the Megaplex™ RT primers, Human Pool Set v2.0 (Applied Biosystems), and quantitated on an Applied Biosystems 7900 HT Real-Time PCR system. The TLDA data were processed using the accompanying software RQ Manager (Applied Biosystems). For each sample, the normalization factor was calculated as a mean of the two endogenous controls, RNU44 and RNU48. ΔC_{q} was calculated for each miRNA as (C_{q}(miRNA)-normalization factor). All protocols followed were as per the manufacturer's recommendation.

Oligonucleotide Transfections:

Freshly dissociated human airway epithelial cells or immortalized cell lines were transfected in pre-coated 96 well plates (Costar) or Transwell™ Permeable Supports (0.33 cm² 0.4 μm polyester membrane, Costar 3470). Lipofectamine™ RNAiMAX (Invitrogen) was used as a reverse transfection reagent. Pre-coated (with human placental collagen Type IV, Sigma) substrates were incubated with the transfection mix comprising of Opti-MEM (Invitrogen), oligonucleotide (Integrated DNA Technologies) and Lipofectamine™ RNAiMAX (Invitrogen). 15-20 minutes later, 200,000 freshly dissociated cells suspended in DMEM/F-12 were added to each well/insert. Between 4-6 hrs later, all media from the apical surface was aspirated and complete media added to the basolateral surface. Media on the basolateral surface were changed every 3-4 days. For human primary epithelial cultures, USG media described above was used. For cultures from immortalized cell lines: Calu-3, CFBE41o -(termed CFBE throughout) (Kunzelmann et al. Am. J. Respir. Cell Mol. Biol. 8, 522 (1993)), complete media specific to each cell line was used (Calu-3: MEM (Gibco)+10% FBS (Atlanta Biologicals)+1% Pen Strep (Gibco); CFBE: Advanced DMEM (Gibco)+1% L-Glutamine (Gibco)+10% FBS (Atlanta Biologicals)+1% Pen Strep (Gibco)).

Oligonucleotide Reagents:

The DsiRNAs were designed (Kim, D. H. et al. Synthetic dsRNA Dicer substrates enhance RNAi potency and efficacy. Nature Biotechnol. 23, 222-226 (2005); Rose, S. D. et al. Functional polarity is introduced by Dicer processing of short substrate RNAs. Nucleic Acids Res. 33, 4140-4156 (2005)), synthesized and validated (Behlke, M. A. Chemical modification of siRNAs for in vivo use. Oligonucleotides 18, 305-319 (2008); Collingwood, M. A. et al. Chemical modification patterns compatible with high potency dicer-substrate small interfering RNAs. Oligonucleotides 18, 187-200 (2008)) by Integrated DNA Technologies. The miRNA-mimic (Behlke, M. A. Chemical modification of siRNAs for in vivo use. Oligonucleotides 18, 305-319 (2008); Henry, J. C., Azevedo-Pouly, A. C. & Schmittgen, T. D. microRNA Replacement Therapy for Cancer. Pharm. Res. (2011)) and anti-miRNA (Lennox, K. A. & Behlke, M. A. Chemical modification and design of anti-miRNA oligonucleotides. Gene Ther. (2011); Melkman-Zehavi, T. et al. miRNAs control insulin content in pancreatic beta-cells via downregulation of transcriptional repressors. EMBO J. 30, 835-845 (2011)) were also designed and synthesized by Integrated DNA Technologies. All accompanying control sequences (Scr) were also generated by Integrated DNA Technologies.

r = RNA
m = 2′OMe modification
SS = Sense strand
AS = Antisense strand
* = Phosphorothioate linkages
+ = Locked Nucleic Acid modification
SpC3 = C3 Spacer modification
SIN3A DsiRNA
Sense strand sequence:
(SEQ ID NO: 6)
/5Phos/rGrCrGrArUrArCrArUrGrArArUrUrCrArGrArUrArCr
UrACC
Antisense strand sequence:
(SEQ ID NO: 7)
/5Phos/rGrGrUrArGrUrArUrCrUmGrAmArUrUrCrArUrGrUmAr
UmCrGmCmUmC
CFTR DsiRNA
Sense strand sequence:
(SEQ ID NO: 8)
/5Phos/rGrGrArArGrArArUrUrCrUrArUrUrCrUrCrArArUrCr
CrAAT
Antisense strand sequence:
(SEQ ID NO: 9)
/5Phos/rArUrUrGrGrArUrUrGrAmGrAmArUrArGrArArUrUmCr
UmUrCmCmUmU
Scr (Negative control for DsiRNAs)
Sense strand sequence:
(SEQ ID NO: 10)
/5Phos/rCrGrUrUrArArUrCrGrCrGrUrArUrArArUrArCrGrCr
GrUAT
Antisense strand sequence:
(SEQ ID NO: 11)
/5Phos/rArUrArCrGrCrGrUrArUmUrAmUrArCrGrCrGrArUmUr
AmArCmGmAmC
miR-138 anti-miRNA
(SEQ ID NO: 12)
mC*mG* + G* mCmC + T mGmA + T mUmC + A mCmA + A
mCmA + C mCmA* + G* mC*mU
Scr (negative control for anti-miRNA)
(SEQ ID NO: 13)
mG*mC* + G* mU*mA* + T* mU*mA* + T* mA*mG* + C*
mC*mG* + A* mU*mU* + A* mA*mC* + G* mA
miR-138 mimic
Sense strand sequence:
(SEQ ID NO: 4)
/5SpC3/rCmG rGmC/iSpC3/ mUrGmA rUmUrC mArCmA
rAmCrA mCrCmA rGmCrU
Antisense strand sequence:
(SEQ ID NO: 5)
/5Phos/rArG rCrUrG rGrUrG rUrUrG rUrGrA rArUrC
rArGrG mCmCmG

Specificity of Oligonucleotide Transfections:

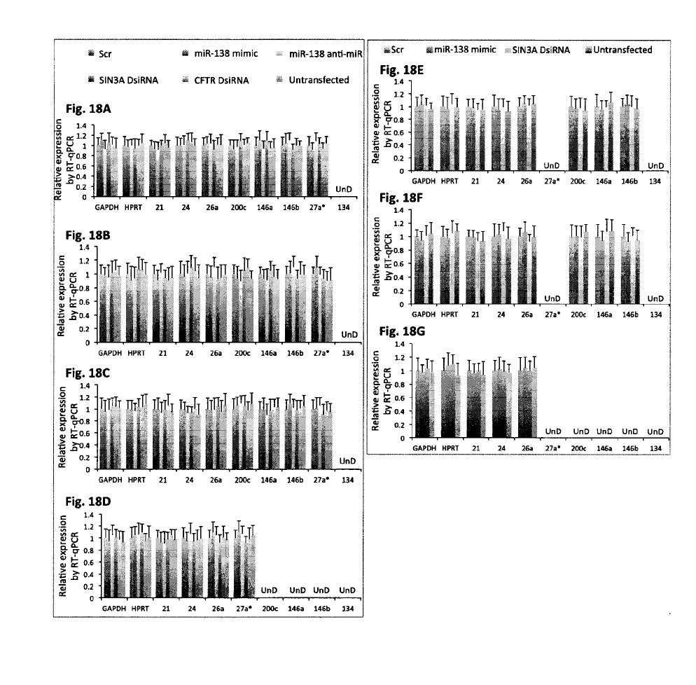

To ascertain the specificity of the following oligonucleotides: CFTR DsiRNA, SIN3A DsiRNA, miR-138 mimic, and miR-138 anti-miRNA, we harvested RNA from cells transfected with these oligonucleotides and measured the expression of multiple genes and miRNAs (FIG. 18). 24 hrs post-transfection, RNA was harvested from each sample and subjected to quantitative RT-PCR for the following genes: SFRS9 (normalizer for mRNA), GAPDH, HPRT, RNU48 (normalizer for miRNAs), miRs-21, -24, -26a, -200c, -146a, -146b, -27a*, -134.

Quantitative RT-PCR (RT-qPCR):

First-strand cDNA was synthesized using SuperScript® II (Invitrogen), and oligo-dT and random-hexamer primers. Sequence specific PrimeTime® qPCR Assays for human CFTR, SIN3A, GAPDH, HPRT, and SFRS9 were designed and validated (Integrated DNA Technologies). To quantitate miRNAs, TaqMan® microRNA Assays (Applied Biosystems) were obtained for miR-138, RNU48 (control) and 8 other miRNAs (negative control, miRs-21, -24, -26a, -200c, -146a, -146b, -27a*, -134). All reactions were setup using TaqMan® Fast Universal PCR Master Mix (Applied Biosystems) and run on the Applied Biosystems 7900 HT Real-Time PCR system. All experiments were performed in quadruplicate. mRNA and miRNA quantification in cell lines represents 8 independent transfections in 4 separate experiments. mRNA quantification in human primary airway epithelial cultures represent 8 independent transfections in 8 non-CF donors and 4 CF donors.

/56-FAM/: single isomer 6-carboxyfluorescein
/3IABkFQ/: Iowa Black FQ = dark quencher
CFTR:
Forward-
(SEQ ID NO: 14)
CAACATCTAGTGAGCAGTCAGG
Reverse-
(SEQ ID NO: 15)
CCCAGGTAAGGGATGTATTGTG
Probe-
(SEQ ID NO: 16)
/56-FAM/TCCAGATCCTGGAAATCAGGGTTAGT/3IABkFQ/
SIN3A:
Forward-
(SEQ ID NO: 17)
GCACAGAAACCAGTATTTCTCCC
Reverse-
(SEQ ID NO: 18)
GGTCTTCTTGCTGTTTCCTTCC
Probe-
(SEQ ID NO: 19)
/56-FAM/TGCTCTCGACCACGTTGACACTTCC/3IABkFQ/
GAPDH:
Forward-
(SEQ ID NO: 20)
GGCATGGCCTTCCGTGT
Reverse-
(SEQ ID NO: 21)
GCCCAGGATGCCCTTGAG
Probe-
(SEQ ID NO: 22)
/56-FAM/CCTGCTTCACCACCTTCTTGATGTCATCAT/3IABkFQ/
HPRT:
Forward-
(SEQ ID NO: 23)
GACTTTGCTTTCCTTGGTCAG
Reverse-
(SEQ ID NO: 24)
GGCTTATATCCAACACTTCGTGGG
Probe-
(SEQ ID NO: 25)
/56-FAM/ATGGTCAAGGTCGCAAGCTTGCTGGT/3IABkFQ/
SFRS9:
Forward-
(SEQ ID NO: 26)
TGTGCAGAAGGATGGAGT
Reverse-
(SEQ ID NO: 27)
CTGGTGCTTCTCTCAGGATA
Probe-
(SEQ ID NO: 28)
/56-FAMITGGAATATGCCCTGCGTAAACTGGA/3IABkFQ/
Primers to distinguish between endogenous CFTR and
transgene CFTR-HA:
Endogenous CFTR: Forward-
(SEQ ID NO: 29)
AGTGGAGGAAAGCCTTTGGAGT
Endogenous CFTR: Reverse-
(SEQ ID NO: 30)
ACAGATCTGAGCCCAACCTCA
CFTR-HA: Forward-
(SEQ ID NO: 31)
CCCATATGATGTGCCTGATT
CFTR-HA: Reverse-
(SEQ ID NO: 32)
GTCGGCTACTCCCACGTAAA

Electrophysiology Studies:

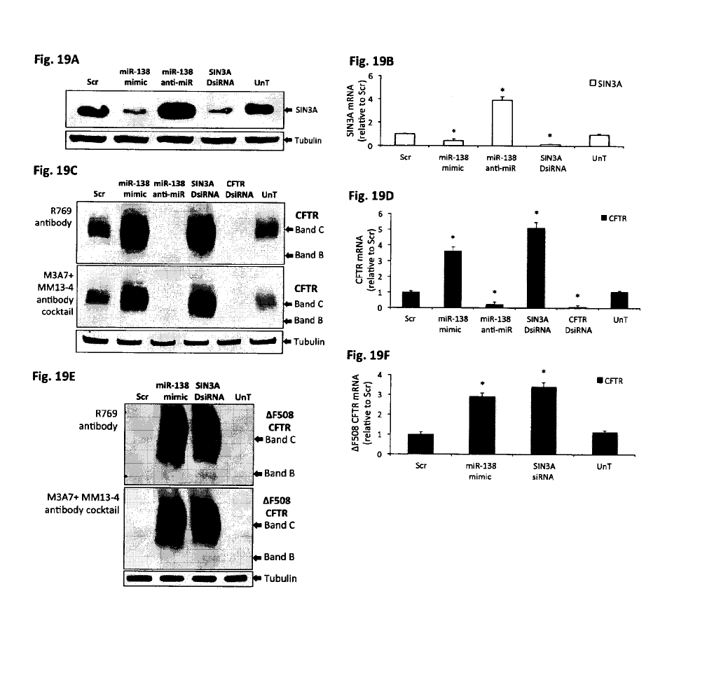

Transepithelial Cl⁻ current measurements were made in Ussing chambers about 2 weeks post-seeding (Itani, 0. A. et al. Human cystic fibrosis airway epithelia have reduced Cl− conductance but not increased Na+ conductance. Proc. Natl. Acad. Sci. USA 108, 10260-10265 (2011)). Briefly, primary cultures were mounted in a modified Ussing chamber (Jim's Instruments, 8 wells per instrument). Transepithelial Cl⁻ current was measured under short-circuit current conditions. Cultures were incubated overnight with 10 μM forskolin and 100 μM 3-isobutyl-1-methylxanthine (IBMX). After measuring baseline current, the transepithelial current (Iₜ) response to sequential apical addition of 100 μM amiloride (Amil), 100 μM 4,4′-diisothiocyanoto-stilbene-2,2′-disulfonic acid (DIDS), 4.8 mM [Cl⁻], 10 μM forskolin and 100 μM 3-isobutyl-1-methylxanthine (IBMX), and 100 μM GlyH-101 was measured. Studies were conducted with a Cl⁻ concentration gradient containing 135 mM NaCl, 1.2 mM MgCl₂, 1.2 mM CaCl₂, 2.4 mM K₂PO₄, 0.6 mM KH₂PO₄, 5 mM dextrose, and 5 mM Hepes (pH 7.4) on the basolateral surface, and gluconate substituted for Cl⁻ on the apical side. Transepithelial current measurements were made in 24 Calu-3 ALI cultures, 6 each from four independent experiments, pre-transfected with reagents noted; 3 ALI cultures per condition in human primary airway epithelial cultures (CFTR Q493X/S912X); 8 ALI cultures per condition in human primary airway epithelia donors (wild-type CFTR, CFTR ΔF508/ΔF508, CFTR ΔF508/3659DC, CFTR ΔF508/R1162X). To confirm that the effects of oligonucleotide transfections persisted at the time of conducting the Ussing chamber studies, RT-qPCR and immunoblots measuring SIN3A and CFTR expression in Calu-3 cells (FIG. 19a, 19b) and CFBE cells (FIG. 19c) were performed 14 days post-transfection.

Dual-Luciferase Reporter Assay:

The 3′UTR of SIN3A was cloned into the Xho1/Not1 restriction enzyme sites in the 3′UTR of Renilla luciferase in the psiCHECK™-2 vector (Promega). HEK293T cells were cotransfected with 20 ng of psiCHECK-2 vector and different concentrations of miR-138 mimic. The Lipofectamine™ RNAiMAX (Invitrogen) reverse transfection protocol was used as described above. The miR-138 binding sites on the SIN3A 3′UTR were mutated using the site-directed, ligase-independent mutagenesis (SLIM) protocol (Chiu, J., Tillett, D., Dawes, I. W. & March, P. E. Site-directed, Ligase-Independent Mutagenesis (SLIM) for highly efficient mutagenesis of plasmids greater than 8 kb. J Microbiol. Methods 73, 195-198 (2008); Chiu, J., March, P. E., Lee, R. & Tillett, D. Site-directed, Ligase-Independent Mutagenesis (SLIM): a single-tube methodology approaching 100% efficiency in 4 h. Nucleic Acids Res. 32, (2004)). A plasmid with the scrambled miR-138 binding seed sequence was also cotransfected into HEK293T cells with different concentrations of miR-138 mimic using the Lipofectamine™ RNAiMAX reverse transfection protocol. The Luciferase Assay Reagent (Promega) was used to measure knockdown of Renilla luciferase with the SIN3A 3′UTR (wild type or scrambled) downstream in response to the miR-138 mimic. Renilla luciferase expression was normalized to firefly luciferase.

Primer sequences to amplify SIN3A 3′UTR:
(SEQ ID NO: 33)
F- AAGTTTAAACCTGCAAAGCCAGAGC
(SEQ ID NO: 34)
R- TTGCGGCCGCTTAAGTAAGAACCAAGC
SLIM primers for mutating miR-138 binding sites in
the SIN3A 3′UTR:
First miR-138 binding site
(SEQ ID NO: 35)
FS- GAGCTAAGACTGGAGTCTCC
(SEQ ID NO: 36)
RS - TGTGCAAGCAAACTGCATGTC
(SEQ ID NO: 37)
FT-GTTTGCTTGCACACGTTAATCGAGCTAAGACTGGAGTCTCCTGTGGC
CTAACTTTCAATG
(SEQ ID NO: 38)
RT - CATTGAAAGTTAGGCCACAGGAGACTCCAGTCTTAGCTCGATTAA
CGTGTGCAAGCAAAC
Second miR-138 binding site
(SEQ ID NO: 39)
FS - TTTACTCTCTGACACACACACG
(SEQ ID NO: 40)
RS - GATGGCACTAAGGTAGAC
(SEQ ID NO: 41)
FT - GTCTACCTTAGTGCCATCCGTTAATTTTACTCTCTGACACACACA
CG
(SEQ ID NO: 42)
RT - CGTGTGTGTGTCAGAGAGTAAAATTAACGGATGGCACTAAGGTAG
AC

SDS-PAGE and Immunoblotting

(Wang, X. et al. Hsp90 cochaperone Aha1 downregulation rescues misfolding of CFTR in cystic fibrosis. Cell 127, 803-815 (2006)): Cell lines or primary cultures were washed with PBS and lysed in freshly prepared lysis buffer (1% Triton, 25 mM Tris pH 7.4, 150 mM NaCl, protease inhibitors (cOmplete™, mini, EDTA-free, Roche)) for 30 min at 4° C. The lysates were centrifuged at 14,000 rpm for 20 min at 4° C., and the supernatant quantified by BCA Protein Assay kit (Pierce). 20 μg (Calu-3) and 50 μg (human primary airway epithelial cultures, HeLa, HEK293T) of protein per lane was separated on a 7% SDS-PAGE gel for western blot analysis. Antibodies were procured for SIN3A (1:1000, R&D Systems), CTCF (1:500, Cell Signaling Technology), CFTR (R-769 (1:2000, CFFT), MM13-4 (1:1000, Millipore), M3A7 (1:500, Millipore), 24-1 (1:1000, R&D Systems)), hemagglutinin (1; 1000, Covance) and α-tubulin (1:10000, Sigma). Protein abundance was quantified by densitometry using an Alphalnnotech Fluorochem Imager (Alphalnnotech). For CFTR, band B and C were quantified separately. All bands were normalized to α-tubulin. Experiments were performed in triplicates per donor and mean and standard error of the mean determined using unpaired two-tailed t-test. SIN3A and CFTR immunoblots in cell lines shown represent 8 independent transfections pooled. Densitometry measurements in cell lines represents western blots performed in triplicate from 4 separate experiments. SIN3A and CFTR immunoblots in human primary airway epithelial cultures shown represent 8 independent transfections. Densitometry measurements in human primary airway epithelial cultures represent 8 independent transfections in 8 non-CF donors each and 4 CF donors each. Western blots were probed, stripped and re-probed as follows. PVDF membranes were first probed with the R-769 anti-CFTR antibody. After imaging, the PVDF membrane was stripped with Restore Western Blot Stripping Buffer (Thermo Scientific) for 15 minutes, washed in Tris Buffered Saline-Tween (TBS-T) and blocked in 5% Bovine Serum Albumin (BSA, Pierce) for 1 hr. The membrane was washed in TBS-T and incubated with the goat anti-mouse secondary antibody (1:10000, Sigma) for 1 hr and imaged. If signal was detected, the stripping procedure was repeated till no signal was observed. The membrane was washed in TBS-T, blocked for 1 hr in 5% BSA and re-probed with the M3A7+MM13-4 anti-CFTR antibody cocktail or the anti-HA antibody. The following pairs of western blots were probed with R-769, and re-probed with M3A7+MM13-4): FIG. 1 d & FIG. 7a, FIG. 2b, FIG. 8a, FIG. 10b-both panels, FIG. 4b, FIG. 16a, FIG. 17b-both panels, FIG. 19c-both panels, and FIG. 19e-both panels.

Measuring Cell Surface Display of CFTR:

Hela cells stably expressing wild-type CFTR or CFTR-ΔF508 were kindly provided by Dr. G. Lukacs (Sharma, M., Benharouga, M., Hu, W. & Lukacs, G. L. Conformational and temperature-sensitive stability defects of the delta F508 cystic fibrosis transmembrane conductance regulator in post-endoplasmic reticulum compartments. J Biol. Chem. 276, 8942-8950, (2001); Sharma, M. et al. Misfolding diverts CFTR from recycling to degradation: quality control at early endosomes. J Cell Biol. 164, 923-933 (2004)). Cell surface ELISA was performed on these cells (Okiyoneda, T. et al. Peripheral protein quality control removes unfolded CFTR from the plasma membrane. Science 329, 805-810 (2010)) 6 hrs, 12 hrs, and 24 hrs after transfecting with oligonucleotides. HeLa cells were transfected in 96 well plates (Costar) with the SIN3A DsiRNA and miR-138 mimic as described earlier using the Lipofectamine™ RNAiMAX (Invitrogen) recommended reverse transfection protocol. Briefly, the plate containing the cells was moved to a cold room (4° C.), and all media used was ice cold. Cells were washed with PBS, and blocked for 30 min with PBS containing 5% BSA. Anti-HA primary antibody (Covance) was added in 5% BSA-PBS at a 1:1000 concentration for 1 hr. Cells were washed with PBS, and anti-mouse secondary antibody HRP conjugated (Amersham) was added to cells at 1:1000 concentration in 5% BSA-PBS for 1 hr. Cells were washed through, and signal developed using SureBlue Reserve™ TMB Microwell Substrate (KPL). The reaction was stopped and read on a VersaMax™ Microplate Reader (Molecular Devices) at 540 nm using the SoftMax® Prof Software (Molecular Devices). For normalization, cells were lysed and total protein quantitated using the BCA Protein Assay kit (Pierce). The experiment was performed in quadruplicate, and the data presented as a mean±standard deviation of individual data points. Statistical significance between groups was determined using Student's t-test.

Transduction of Human Primary Airway Epithelial Cultures:

Primary airway epithelial cell cultures were transduced with an adenovirus expressing either wild-type CFTR or CFTR-ΔF508 (Zabner, J., Zeiher, B. G., Friedman, E. & Welsh, M. J. Adenovirus-mediated gene transfer to ciliated airway epithelia requires prolonged incubation time. J Virol. 70, 6994-7003 (1996); Sinn, P. L., Shah, A. J., Donovan, M. D. & McCray, P. B., Jr. Viscoelastic gel formulations enhance airway epithelial gene transfer with viral vectors. Am. J Respir. Cell Mol. Biol. 32, 404-410 (2005)) at a MOI of 100. The primary culture insert was inverted, the virus was suspended in 50 μl of DMEM, and added to the basolateral surface of the culture for a period of 4 hrs. The similar step was then repeated for the apical surface. Throughout, the cultures were kept at 37° C. in a 5% CO₂ incubator. For primary airway epithelial cultures from the CF donor (CFTR Q493X/S912X) transfected with oligonucleotides, transduction with the Ad-CFTR-ΔF508 was performed 11 days post-seeding. CFTR immunoblot, RT-qPCR and transepithelial current (Iₜ) measurements were made 14 days post-seeding.

Microarrays:

Calu-3 cells were transfected with SIN3A DsiRNA and miR-138 mimic by reverse transfection as described above. Total RNA was isolated 48 hrs after transfection using the mirVana™ miRNA isolation kit (Ambion), and only samples that had a RIN >7.0 were selected for microarray analysis. Microarrays were performed at the University of Iowa DNA Core². Briefly, RNA samples were processed with the NuGEN WT-Ovation™ Pico RNA Amplification System, v1.0 along with the WT-Ovation™ Exon Module, v1.0 (NuGEN Technologies) according to the manufacturer's recommended protocols. The GeneChip® Human Exon 1.0 ST Array (Affymetrix) was used to probe the samples. Arrays were scanned using the Affymetrix Model 3000 (7G) scanner and the data collected using the GeneChip® Operating Software (GCOS), v.1.4. Data analysis was performed on Partek® Genomics Suite™ (Partek) using the one-way ANOVA and Student's t-test to determine differentially expressed genes.

Iodide Efflux Assay:

Iodide efflux measurements in HeLa cells were made using a protocol adapted by Lukacs and colleagues (Sharma, M., Benharouga, M., Hu, W. & Lukacs, G. L. Conformational and temperature-sensitive stability defects of the delta F508 cystic fibrosis transmembrane conductance regulator in post-endoplasmic reticulum compartments. J Biol. Chem. 276, 8942-8950, (2001); Glozman, R. et al. N-glycans are direct determinants of CFTR folding and stability in secretory and endocytic membrane traffic. J Cell Biol. 184, 847-862, (2009)). Briefly, HeLa cells were transfected with oligonucleotides in 24 well plates (Costar), and the assay was performed 48 hrs post-transfection (8 wells per condition). As controls, HeLa cells stably expressing wild-type CFTR were plated in 24 well plates (4 wells for cAMP induction and 4 wells for DMSO mock). Cells were observed prior to the experiment to ensure ˜90% confluence. Wells were washed thrice with 2 ml loading buffer, and incubated in 2 ml loading buffer for 1 hr. Wells were washed 7 times in 5 min with 200 μl efflux buffer. 200 μl of efflux buffer was added to each well with a repeat pippetor, and aspirated after 30 sec and stored. After 8 minutes, wells designated for the DMSO control received efflux buffer containing DMSO. Wells designated as test received efflux buffer containing 10 μM forskolin and 100 μM IBMX. 12 such washes were performed in as many minutes. Iodide concentrations in the samples stored were read using iodide selective electrodes that were calibrated with a standard curve.

Chromatin Immunoprecipitation (ChIP):

ChIP was carried out using the EZ-ChIP kit from Millipore (Upstate Protocol). Human primary airway epithelial cells were grown on 150 mm dishes and 5×10⁷ cells were used. Cells were crosslinked with 1% formaldehyde for 10 min and reaction stopped with 0.125 M glycine. Cells were washed with PBS and lysed in 1 ml of 1% SDS, 10 mM EDTA, 50 mM Tris/HCl (pH 8.1) with protease inhibitors. Sample was sonicated to generate fragments under 500 bp. Immunoprecipitation was performed overnight at 4° C. with the SIN3A antibody (Santa Cruz Biotechnology). Manufacturer's recommended protocol were followed with modifications (Blackledge, N. P. et al. CTCF mediates insulator function at the CFTR locus. Biochem. J 408, 267-275, (2007); Blackledge, N. P., Ott, C. J., Gillen, A. E. & Harris, A. An insulator element 3′ to the CFTR gene binds CTCF and reveals an active chromatin hub in primary cells. Nucleic Acids Res. 37, 1086-1094, (2009); Das, P. M., Ramachandran, K., vanWert, J. & Singal, R. Chromatin immunoprecipitation assay. Biotechniques 37, 961-969 (2004); Fowler, A. M., Solodin, N. M., Valley, C. C. & Alarid, E. T. Altered target gene regulation controlled by estrogen receptor-alpha concentration. Mol. Endocrinol. 20, 291-301 (2006)) and immunoprecipitation from each donor was performed in triplicate. Primer sequences used for amplifying DNase I hypersensitive sites (DHS) regions 17a DHS (normalizer), −20.9 DHS, +6.8 DHS and +15.6 DHS (negative control) were obtained from the literature (Blackledge, N. P. et al. CTCF mediates insulator function at the CFTR locus. Biochem. J 408, 267-275, (2007); Blackledge, N. P., Ott, C. J., Gillen, A. E. & Harris, A. An insulator element 3′ to the CFTR gene binds CTCF and reveals an active chromatin hub in primary cells. Nucleic Acids Res. 37, 1086-1094, (2009)). Intron 17a DHS has been reported to not have a putative CTCF binding site or bind CTCF. −20.9 DHS, +6.8 DHS and +15.6 DHS have been shown to have a putative CTCF binding site, but CTCF has been demonstrated to bind only the -20.9 DHS and +6.8 DHS. Additional controls used were: co-immunoprecipitation of CTCF with an anti-SIN3A antibody (Lutz, M. et al. Transcriptional repression by the insulator protein CTCF involves histone deacetylases. Nucleic Acids Res. 28, 1707-1713 (2000)), ChIP with anti-SIN3A antibody without formaldehyde crosslinking, and ChIP without the use of anti-SIN3A antibody. As a positive control, ChIP with anti-CTCF antibody was performed and enrichment was confirmed at -20.9 kb relative to 17a.

DHS17A
Forward-
(SEQ ID NO: 43)
GGATAGTGCTGCTATTACTAAAGGTTTCT
Reverse-
(SEQ ID NO: 44)
ATGGCAGCTCCAACACATGA
Probe-
(SEQ ID NO: 45)
/56-FAM/TCTGAAGACAACAAGCCAAAGGGACAAATTT/3IABkFQ/
DHS −20.9
Forward-
(SEQ ID NO: 46)
CCGGGATGTTGTTTGAAGCTT
Reverse-
(SEQ ID NO: 47)
TTTAAATAGTTGAATAGAGGACGAGATACTTT
Probe-
(SEQ ID NO: 48)
/56-FAM/ATAGTATTTTCTTCTCTCTTCCTTACCTGCCCTCTGCT/
3IABkFQ/
DHS +15.6
Forward-
(SEQ ID NO: 49)
ATCCATTTTCTTCAAGTCTCTCTCCAT
Reverse-
(SEQ ID NO: 50)
GGAATGAGGATTGTTTATGATTTG
Probe-
(SEQ ID NO: 51)
/56-FAM/CCTCTTTATGGAATCTCCTTTTGATTTGAACTTTGA/
3IABkFQ/
DHS +6.8
Forward-
(SEQ ID NO: 52)
TCTTCTTTCCCATTCACCTTTGTC
Reverse-
(SEQ ID NO: 53)
TTTTGGTTTCATTTATACGCACATC
Probe-
(SEQ ID NO: 54)
/56-FAM/CCATTGCTGATAAAGATTGCTCCTTCTATTATTCCA/
3IABkFQ/

CFTR-Associated Gene Network:

Gene products shown previously to interact with CFTR were curated from published literature (Wang, X. et al. Hsp90 cochaperone Aha1 downregulation rescues misfolding of CFTR in cystic fibrosis. Cell 127, 803-815 (2006); Okiyoneda, T. et al. Peripheral protein quality control removes unfolded CFTR from the plasma membrane. Science 329, 805-810 (2010); Hutt, D. M. et al. Reduced histone deacetylase 7 activity restores function to misfolded CFTR in cystic fibrosis. Nature Chem. Biol. 6, 25-33 (2010); Liekens, A. M. et al. BioGraph: unsupervised biomedical knowledge discovery via automated hypothesis generation. Genome Biol. 12, R57 (2011)) were collated to generate a list of CFTR-associated genes. The complete gene list is presented in Table 2. This list was cross referenced with the differentially expressed genes from the miR-138 mimic or SIN3A DsiRNA intervention in Calu-3 cells and used to assess the enrichment significance for genes influencing CFTR biogenesis. The complete enrichment profile is available in Table 3.

Statistical Analysis:

Data are presented as a mean±standard error of individual data points. Statistical significance between groups was determined using Student's t-test or one-way ANOVA as indicated. A P-value <0.05 was considered significant.

### Example 2

### Connectivity MAP Study

The inventors used the connectivity MAP (CMAP) tool (Lamb J, Crawford E D, Peck D, Modell J W, Blat I C, Wrobel M J, Lerner J, Brunet J P, Subramanian A, Ross K N, Reich M, Hieronymus H, Wei G, Armstrong S A, Haggarty S J, Clemons P A, Wei R, Carr S A, Lander E S, Golub T R. Science. 2006 Sep. 29; 313(5795):1929-35) to identify drugs that might mimic the effects of a SIN3A siRNA or a miR-138 mimic. The inventors generated gene sets from the airway cell line Calu-3 following treatment with the siRNA to SIN3A or the miR-138 mimic. These provide a genetic signature for how these two interventions alter the mRNA transcriptome in favor of enhancing the function of mutant ΔF508 protein. The inventors hypothesized that drug treatments that share similar transcriptome signatures would cause partial recovery of ΔF508 CFTR function when applied to CF epithelial cells.

The CMAP screen identified a candidate list of drugs with scores favorable for modifying ΔF508 CFTR processing: Aminoglutethimide, Biperiden, Diphenhydramine, Rottlerin, Midodrine, Thioridazine, Sulfadimethoxine, neostigmine bromide, Pyridostigmine, pizotifen, tyrophostin (AG-1478), valproic acid, Scriptaid and neomycin. These drugs were screened for “rescue” of CFTR mediated chloride transport in CFBE cells homozygous for the ΔF508 mutation. Briefly, the cells were treated with the indicated drugs for 1-6 days, followed by harvesting of cells, and performance of immunoblotting for CFTR. In comparison to cells treated with vehicle alone, a subset of the identified drugs was found to result in partial recovery in expression of band C CFTR in a ΔF508 mutant cell line (FIG. 20). This is a signature for delivery of the mutant protein to the cell membrane where it may form a partially functional CFTR anion channel. The agents that successfully rescued the CFTR mediated chloride transport were the following: Aminoglutethimide, Biperiden, Diphenhydramine, Rottlerin, Midodrine, Thioridazine, Sulfadimethoxine, neostigmine bromide, Pyridostigmine, pizotifen, tyrophostin (AG-1478), valproic acid, Scriptaid and neomycin.

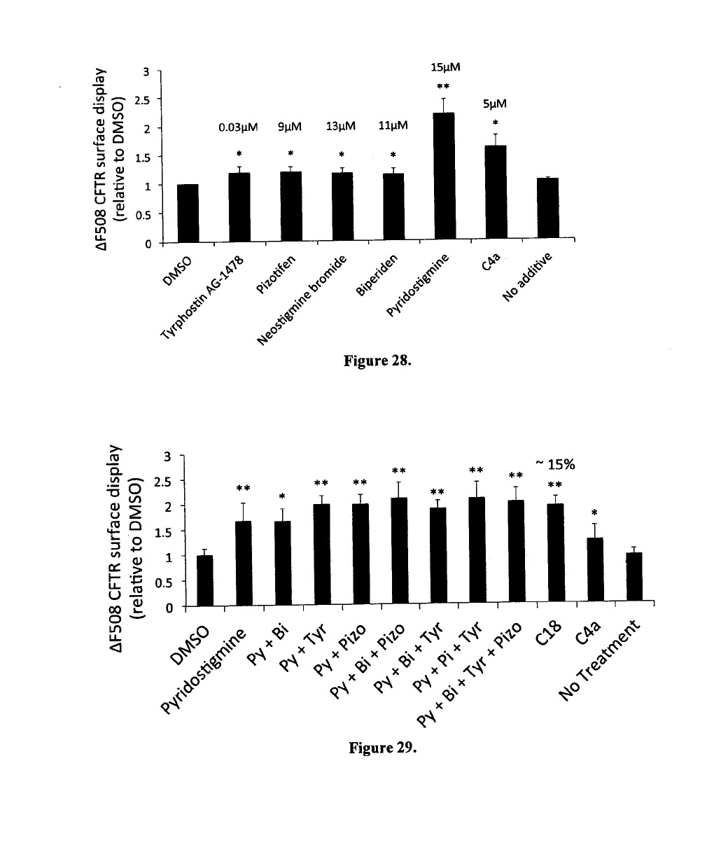

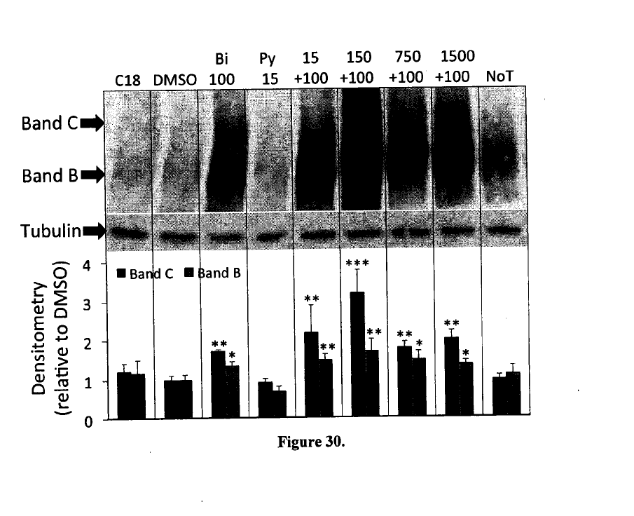

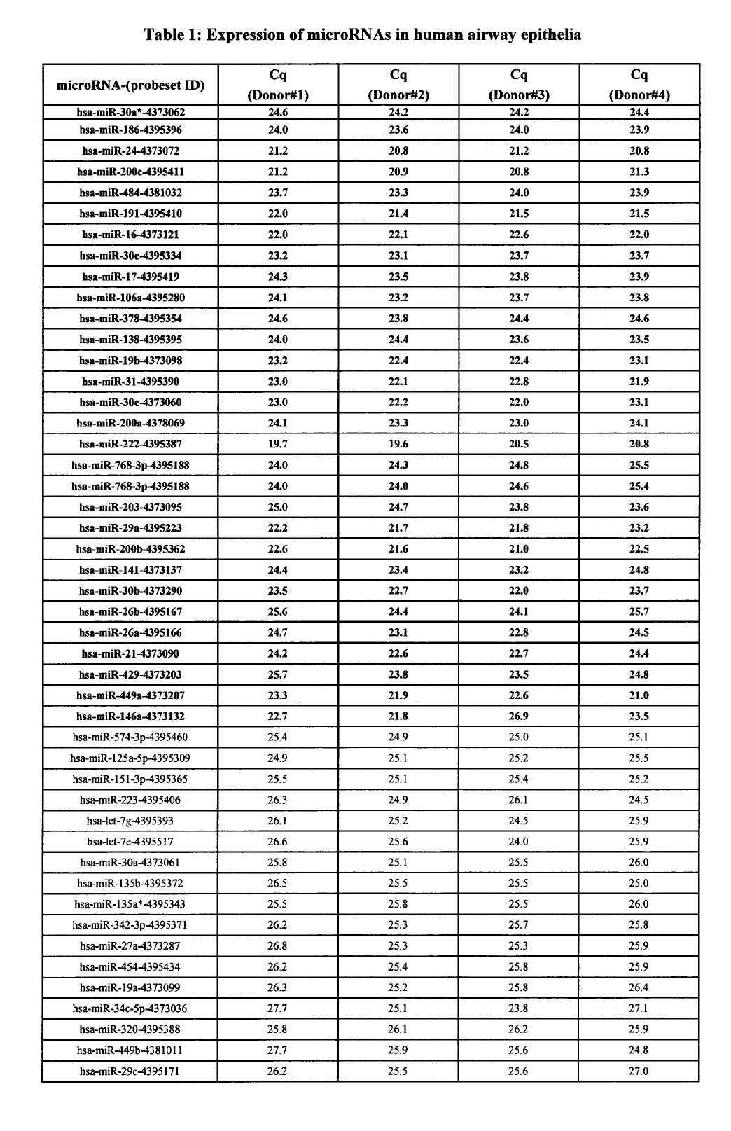

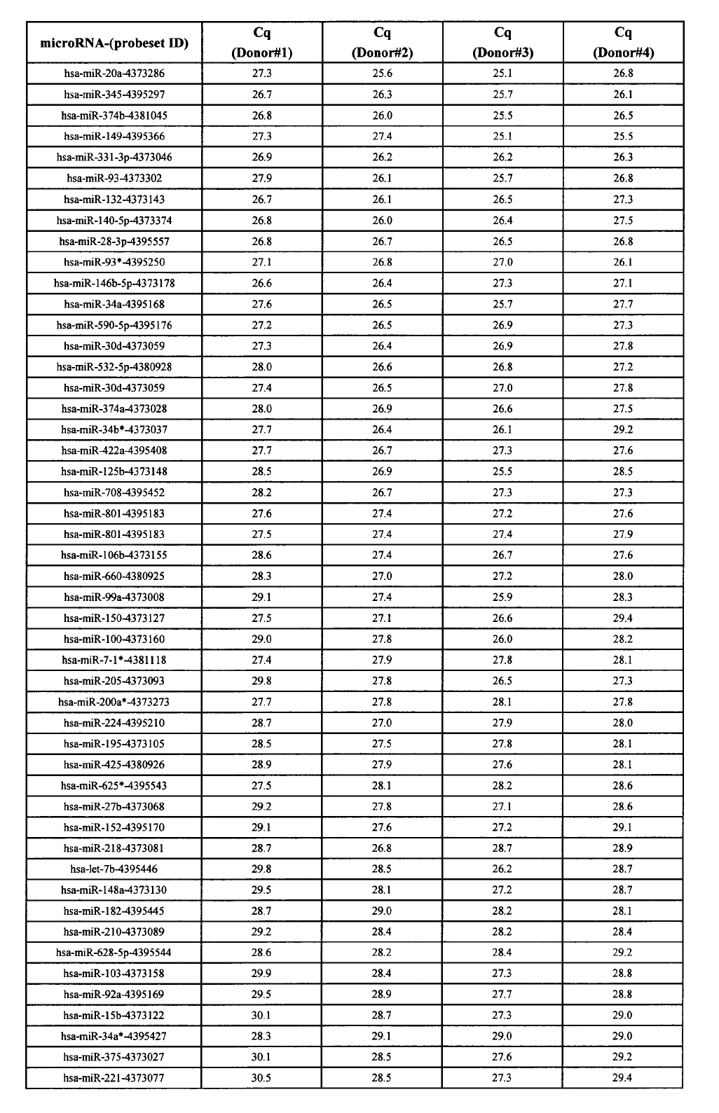

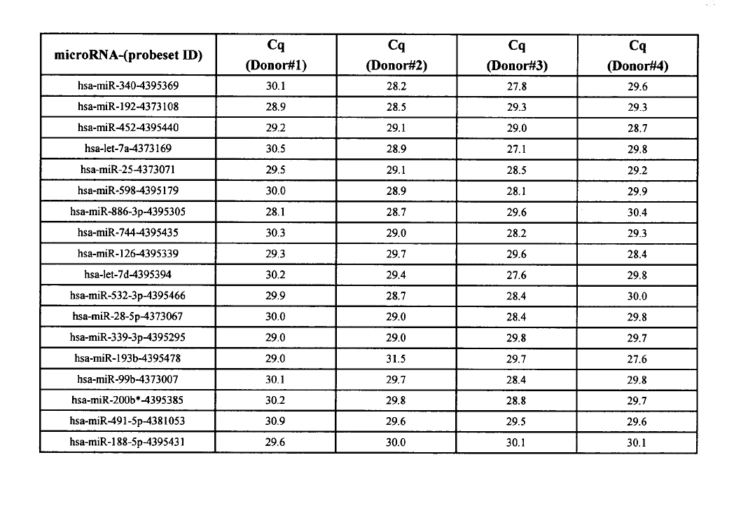

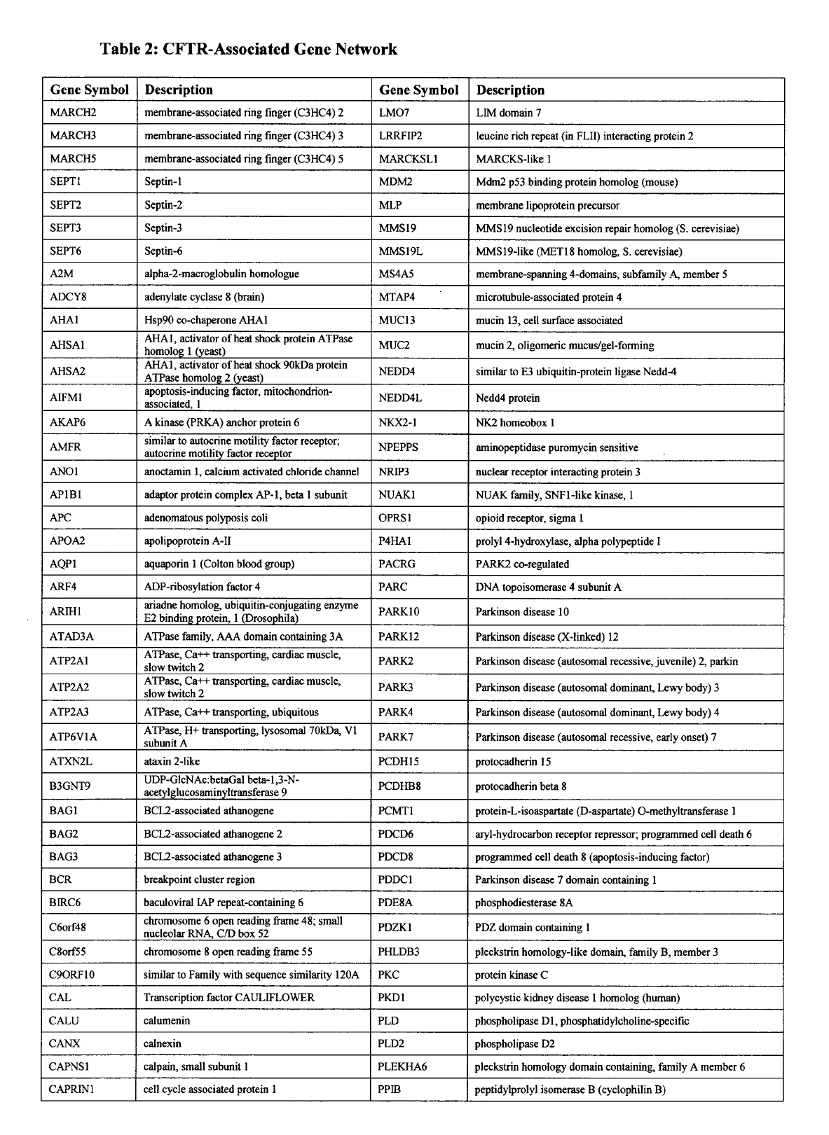

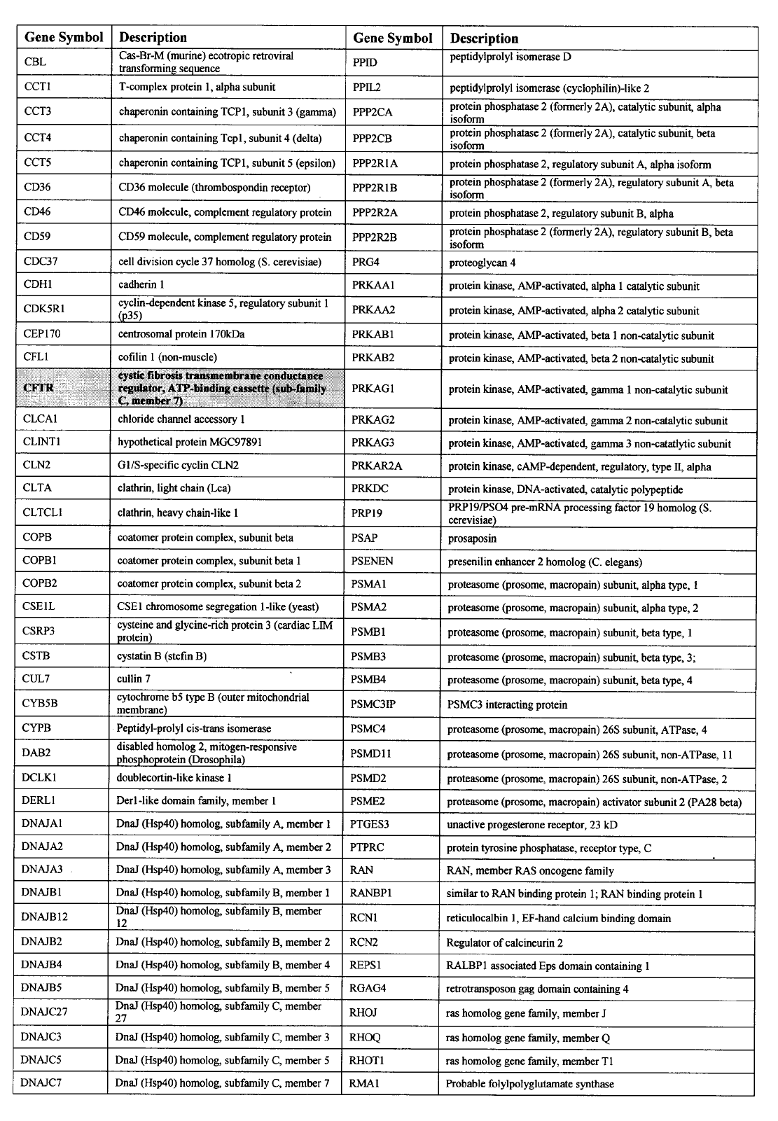

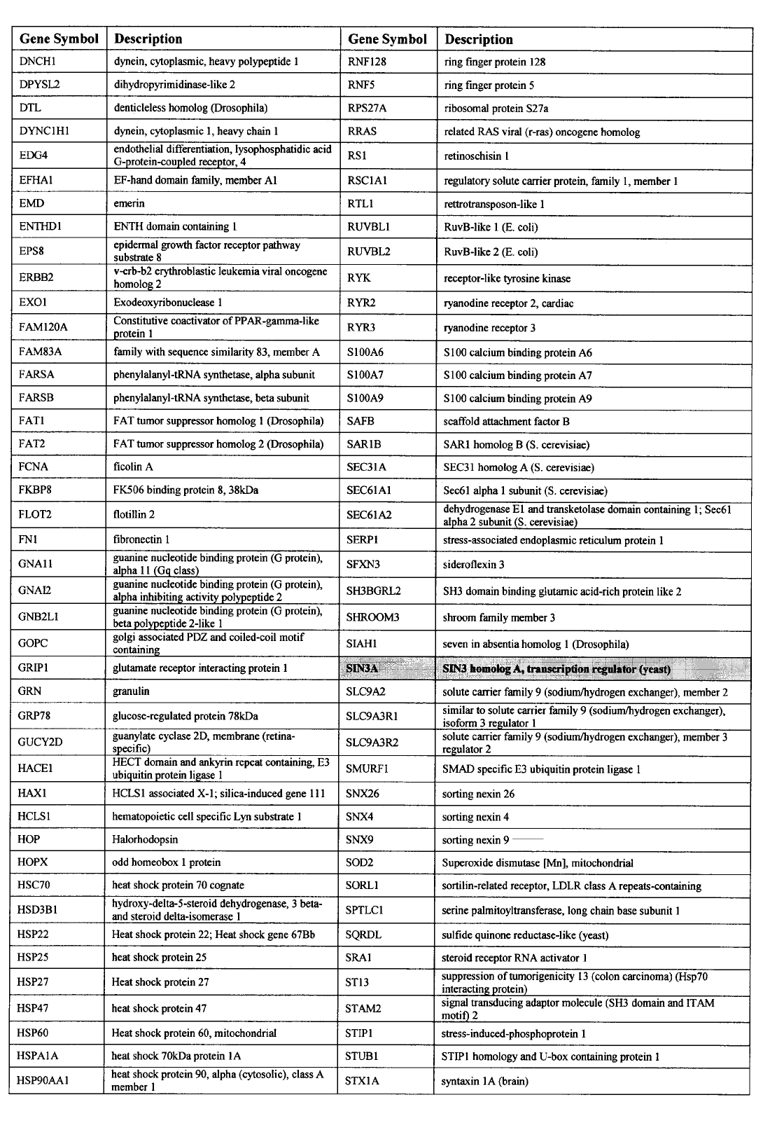

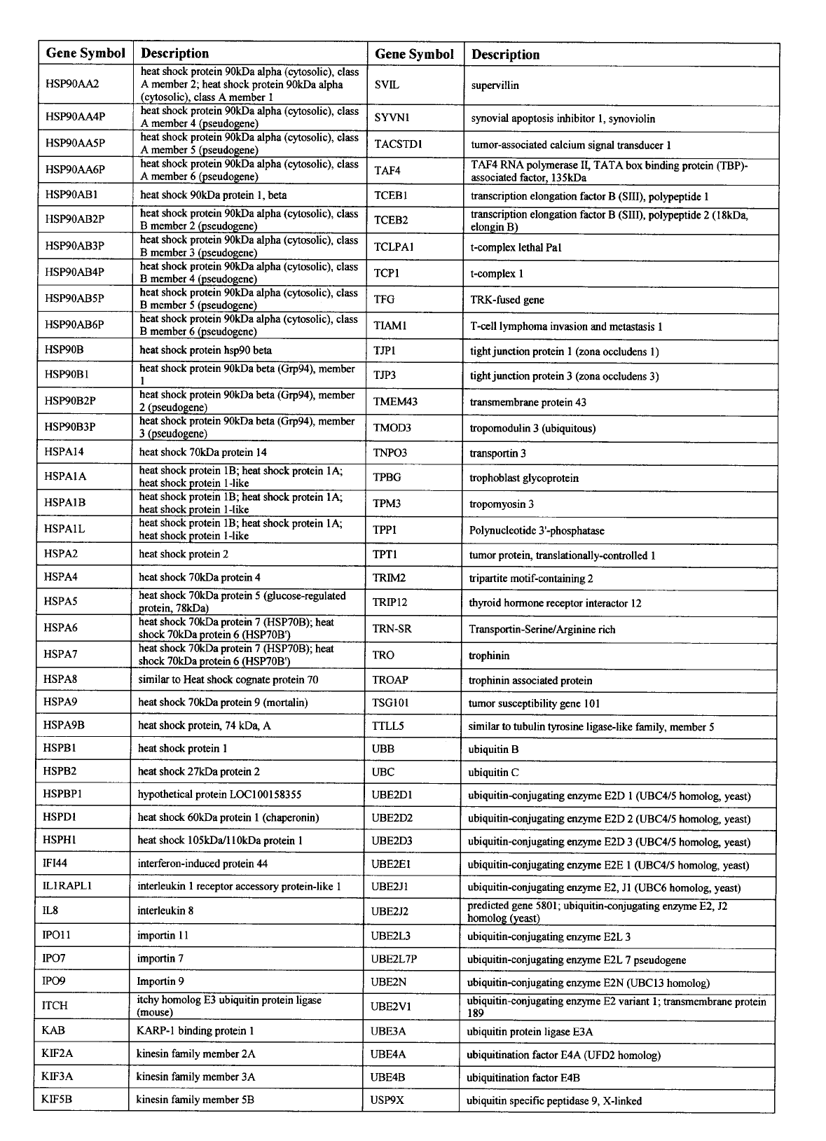

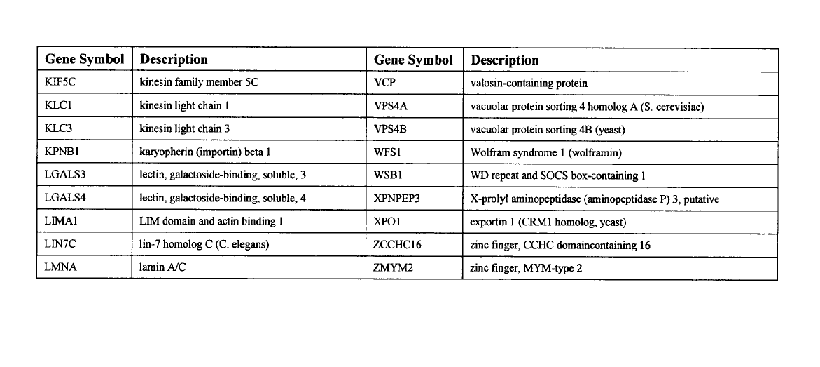

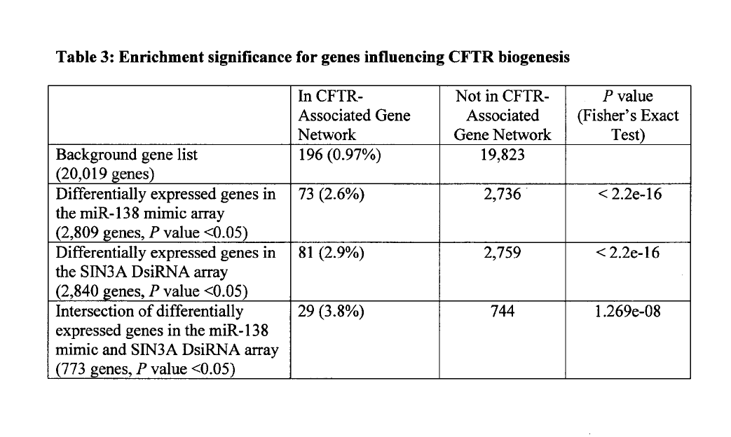

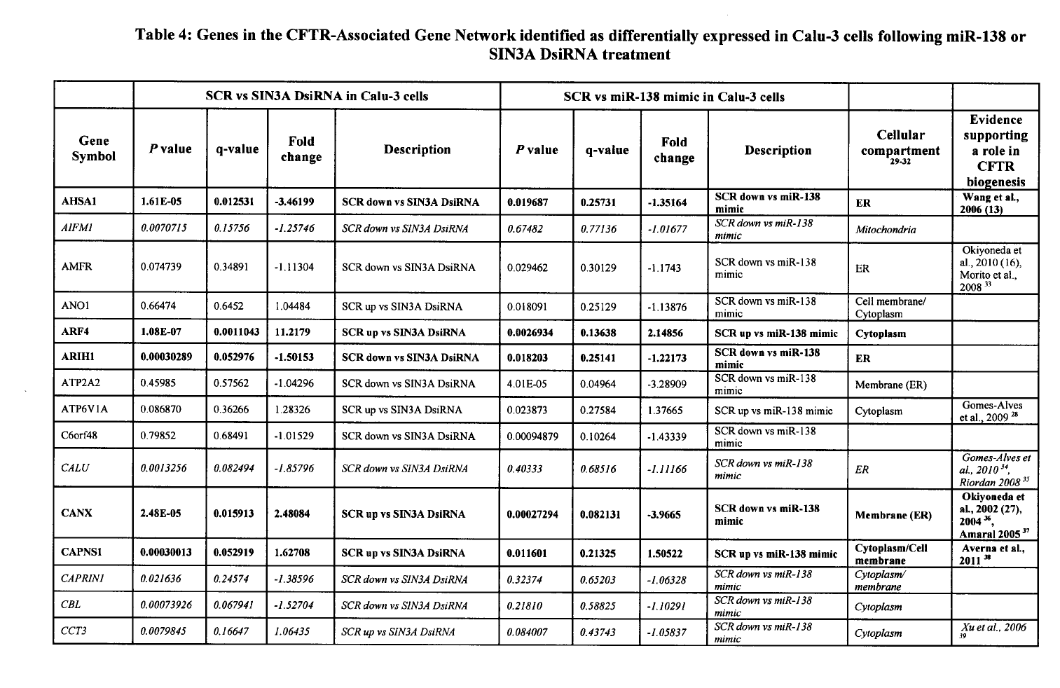

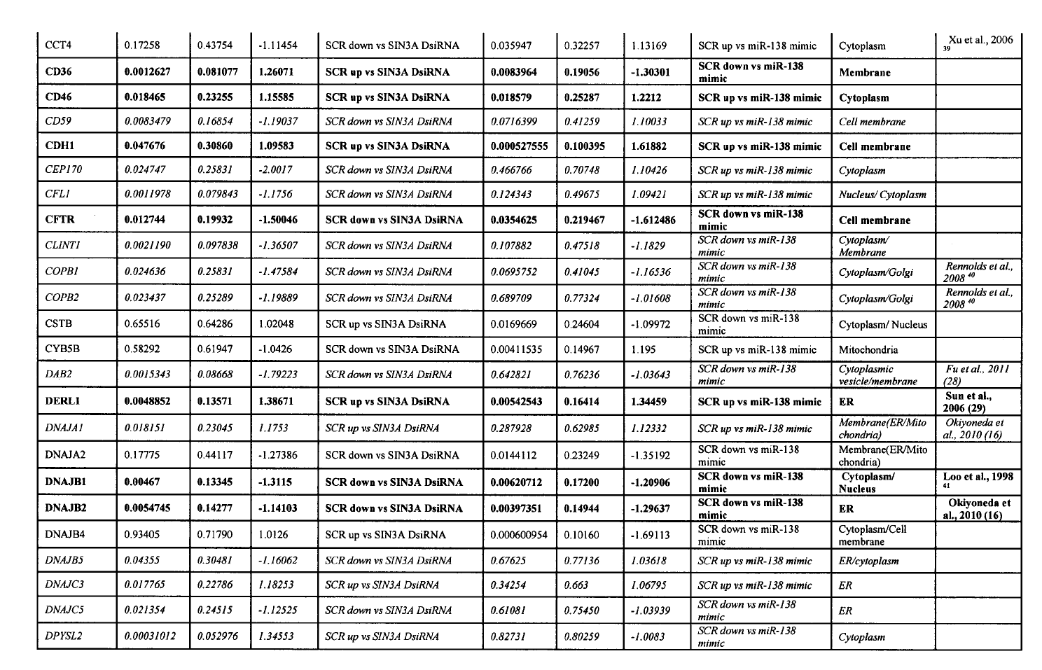

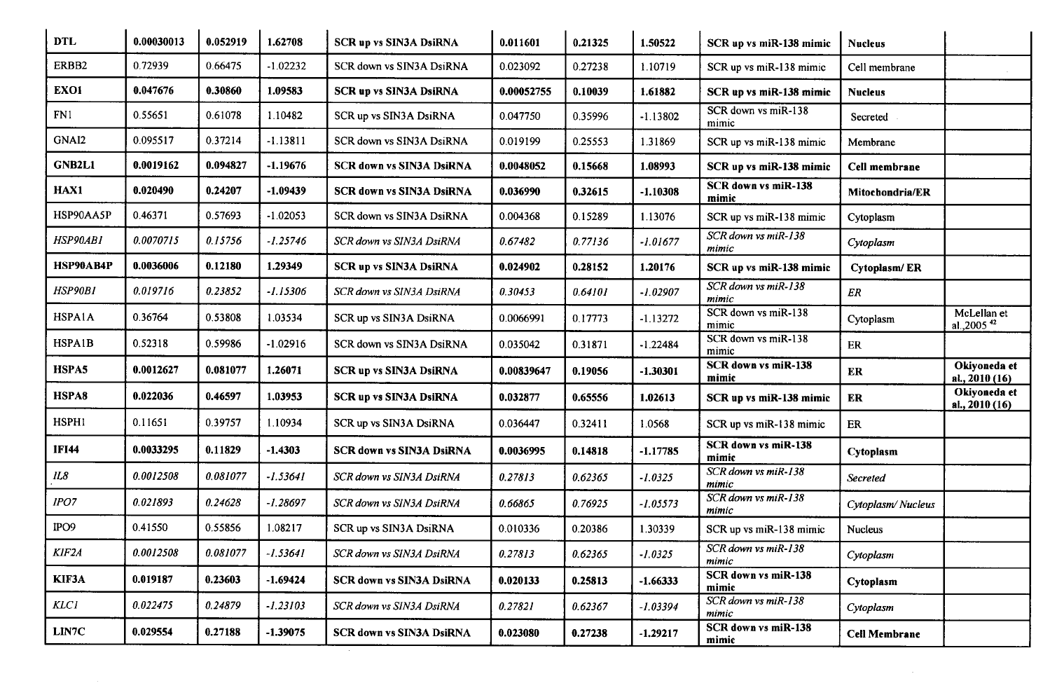

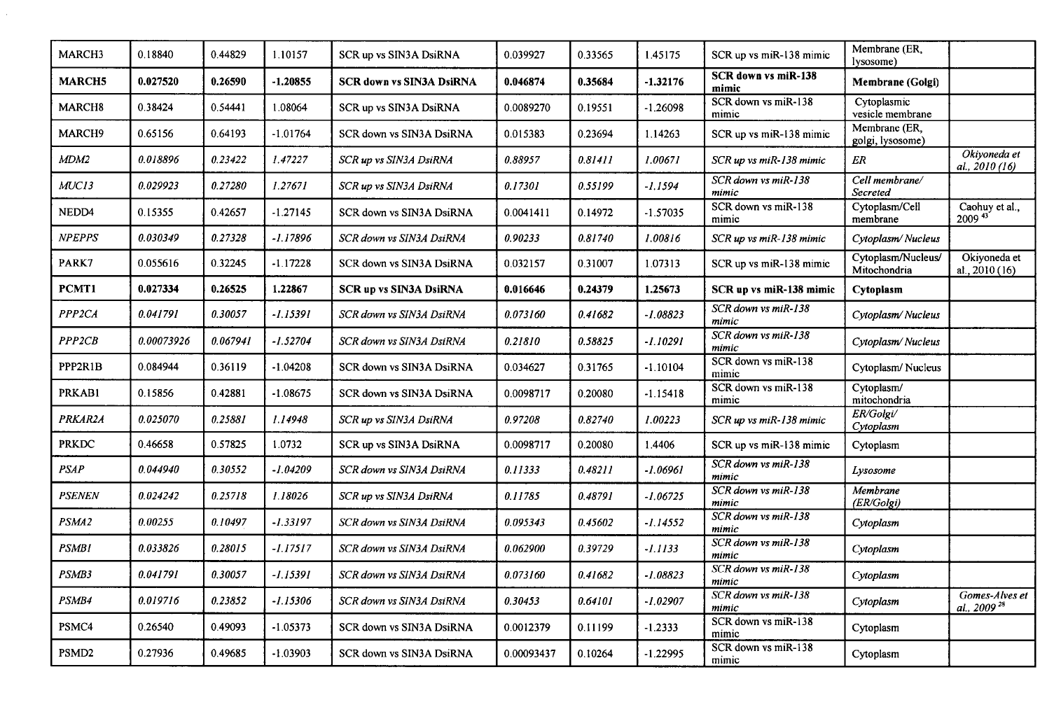

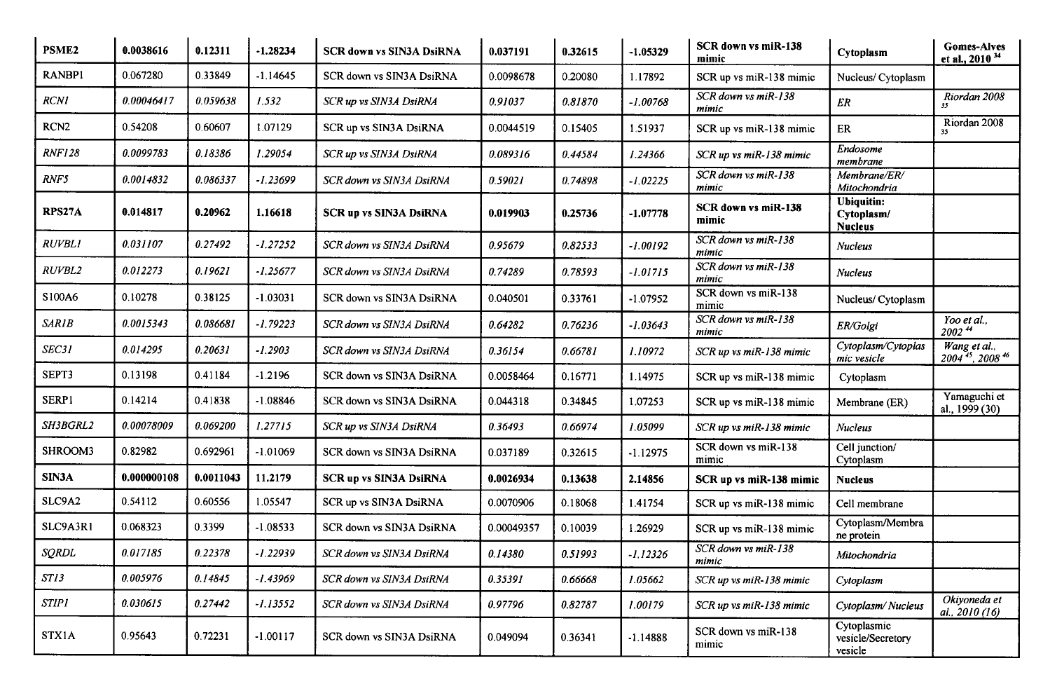

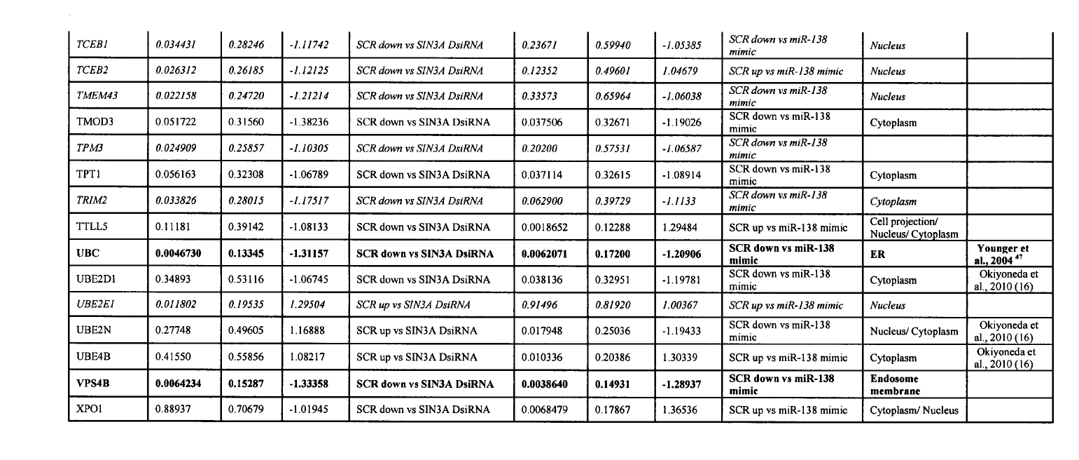

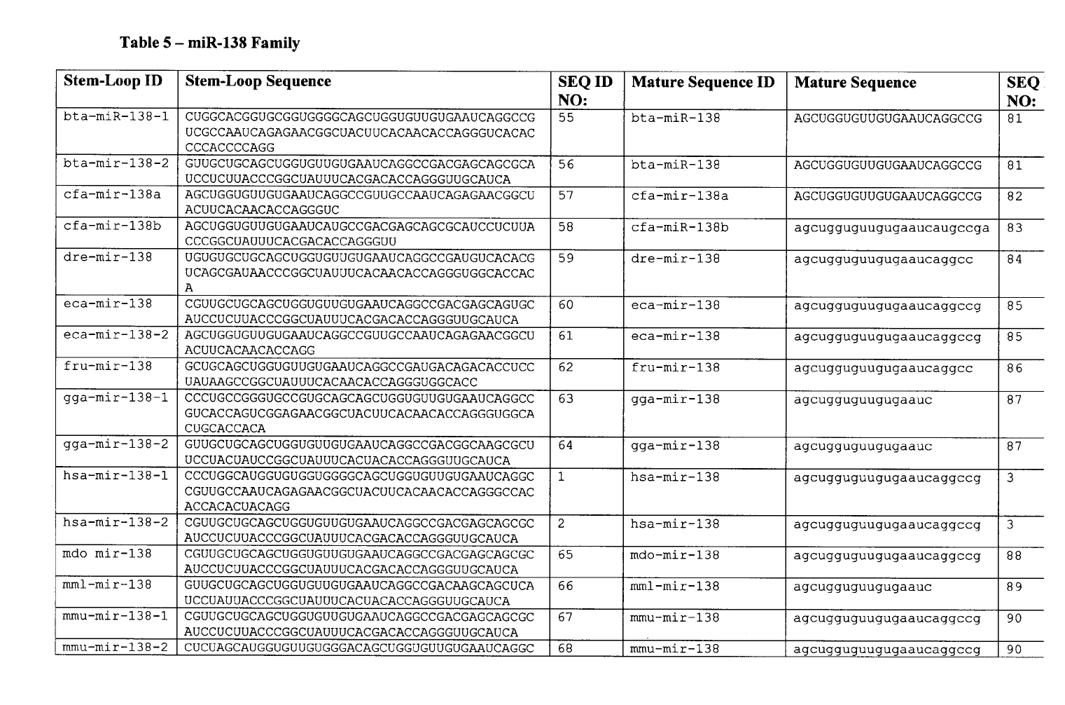

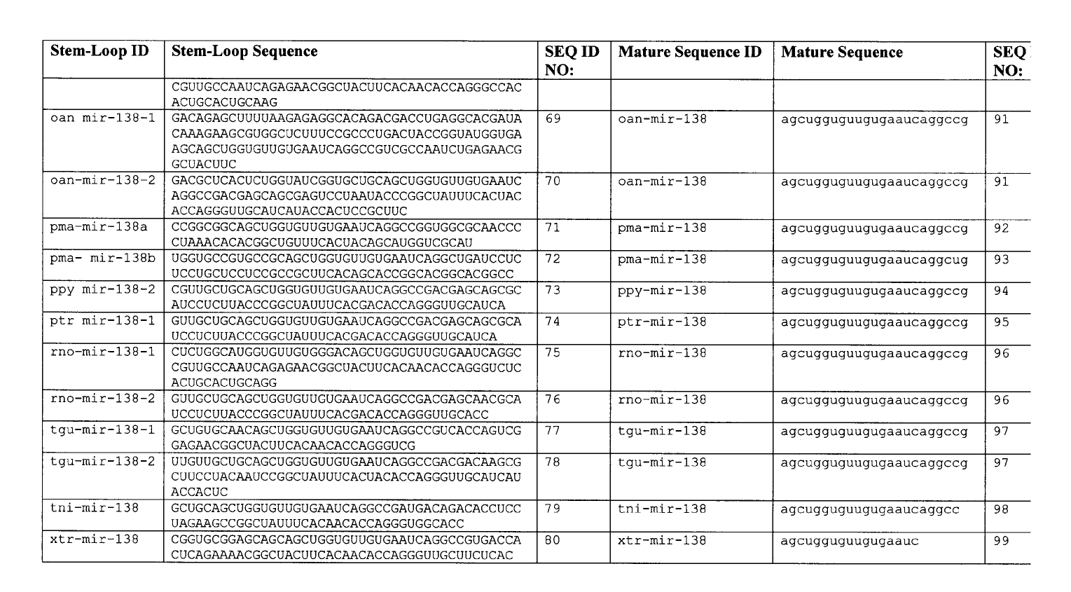

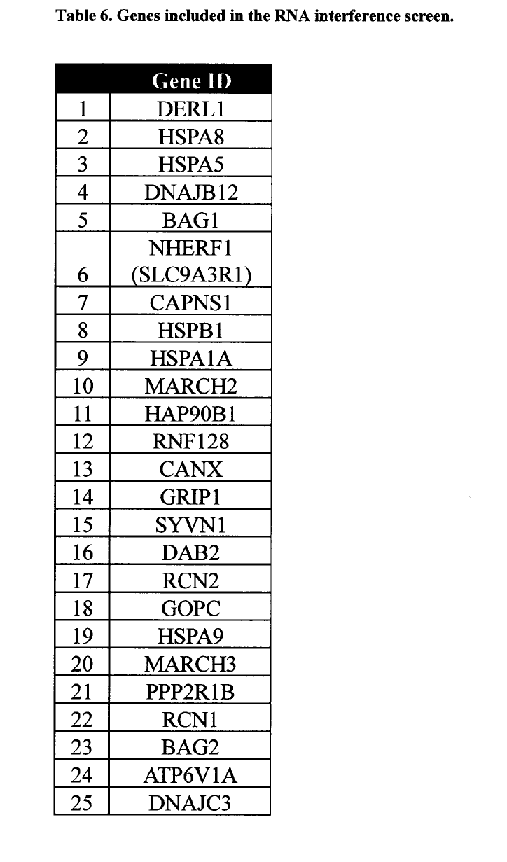

Additional experiments were performed using drugs identified from these CMAP studies. The drugs of interest included tyrphostin AG-1478, pizotifen, neostigmine, pyridostigmine, and biperiden. As show in FIG. 28, each of these individual drug treatments at the indicated concentrations increased CFTR surface display in HeLa cells expressing ΔF508-CFTR-HA. As shown in FIG. 29, these drugs were also tested in combination in HeLa cells expressing ΔF508-CFTR-HA. Combining pyridostigmine with other drugs yielded similar levels of ΔF508-CFTR-HA surface display as seen with the small molecule CFTR corrector compound C18. Furthermore, combining pyridostigmine with biperiden significantly increased ΔF508-CFTR band C abundance in CFBE cells (FIG. 30).

### Example 3

### miR-138 Molecules

The family of miR-138 molecules is a group of microRNA precursors that are found in animals, including humans. The miR-138 precursors are found in numerous tissues, but the mature form is only found in certain cell types. A list of known miR-138 molecules is found in Table 5.

### Example 4

### RNA Interference Screen

From the group of differentially regulated genes in response to SIN3A inhibition or miR-138 mimic treatment of Calu-3 cells, the inventors prioritized candidates for further study using loss of function with RNAi. Table 6 outlines a group of 25 candidates selected from gene products within the CFTR associated gene network (known or suspected interactions during CFTR biogenesis).

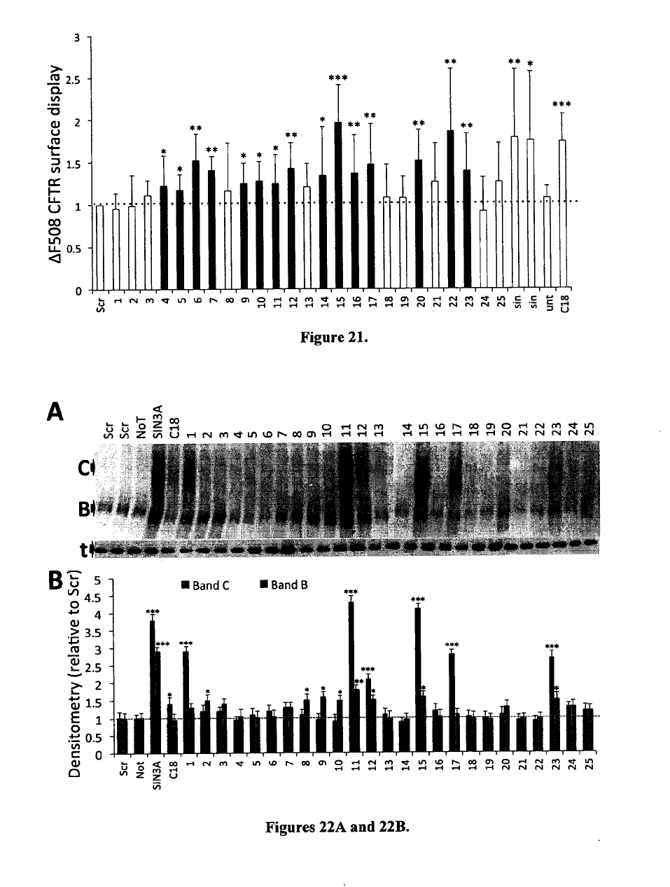

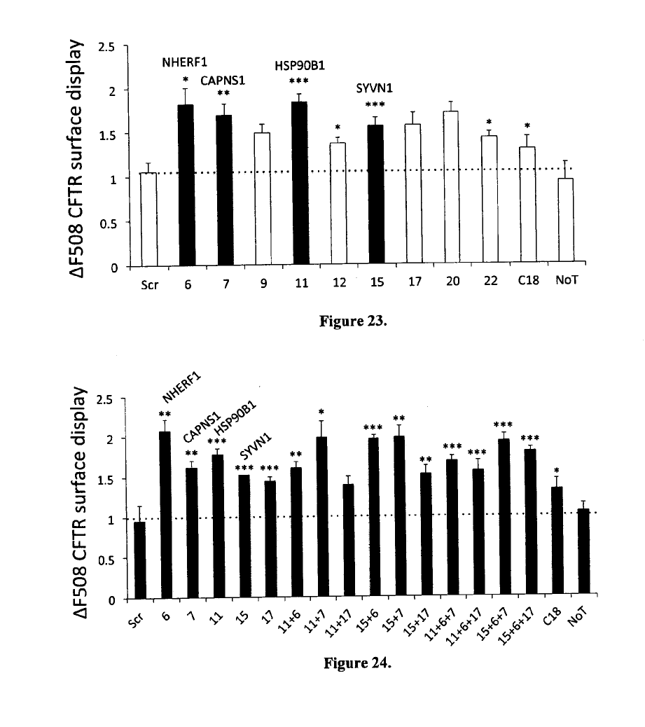

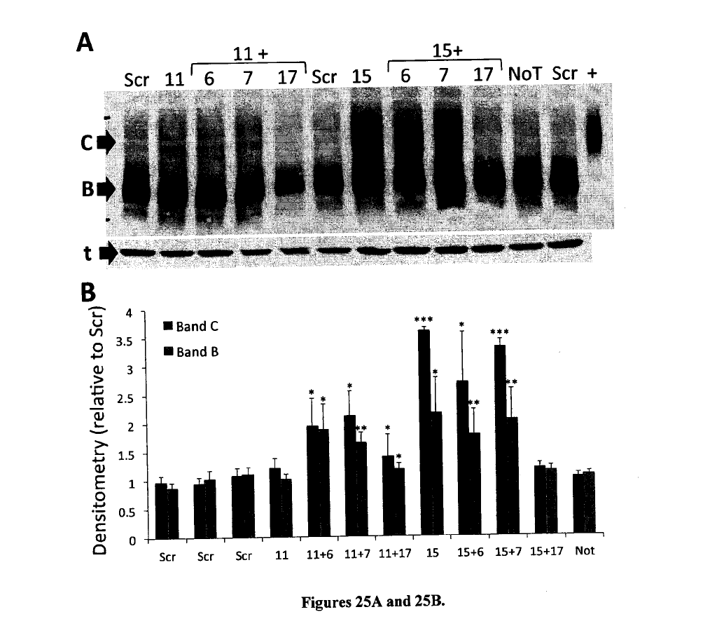

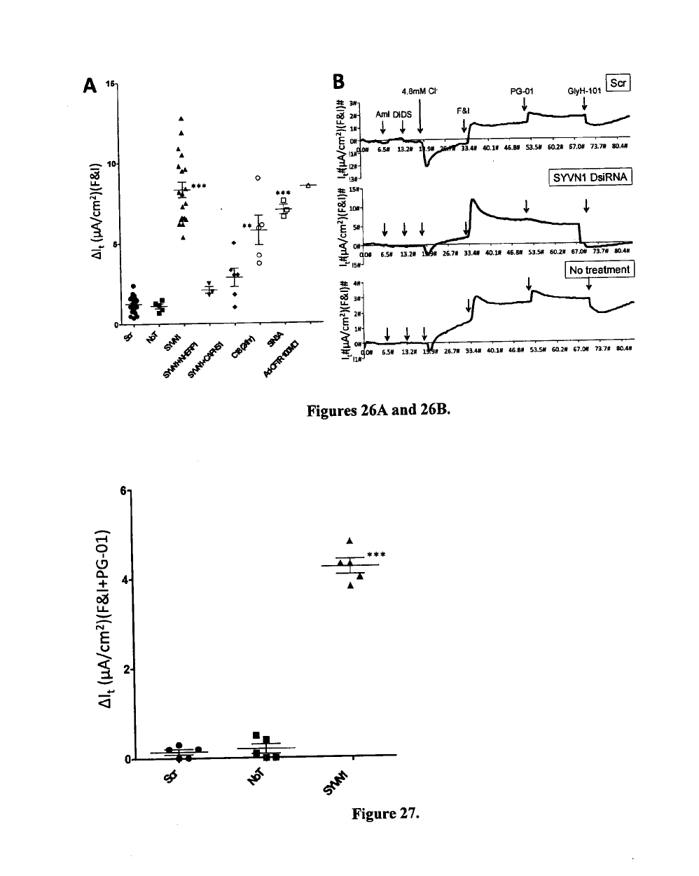

These 25 candidates were further investigated in HeLa cells expressing ΔF508-CFTR-HA (FIG. 21). Knock down of several individual gene products was associated with significantly increased surface display of ΔF508-CFTR protein. Subsequently, CFBE 41o⁻ cells (homozygous for ΔF508-CFTR) were treated with the same interventions shown in Table 6 and ΔF508-CFTR processing was evaluated by immunoblot and the presence of CFTR band C (FIG. 22). Knock down of several genes was associated with significant increases in CFTR band C abundance (as indicated in by * in FIG. 22B). Subsequent replicate experiments further confirmed that inhibition of NHERF1, CAPNS1, HSP90B1, HSP9B1, SYVN1, and RCN1 resulted in ΔF508-CFTR protein trafficking to the cell surface, alone or in combination (FIGS. 23, 24). Additional experiments in CFBE cells demonstrated that the abundance of CFTR band C significantly increased in cells treated with RNAi against HSP90B1 and SYVN1 (FIG. 25). Importantly, inhibition of SYVN1 also significantly increased ΔF508-CFTR mediated Cl⁻ transport in polarized CFBE cells (FIG. 26) and in primary human CF airway epithelia homozygous for the ΔF508 mutation (FIG. 27). These results indicate that manipulating individual gene product involved in CFTR biogenesis may yield therapeutic benefit.

Although the foregoing specification and examples fully disclose and enable the present invention, they are not intended to limit the scope of the invention, which is defined by the claims appended hereto.

All publications, patents and patent applications are incorporated herein by reference. While in the foregoing specification this invention has been described in relation to certain embodiments thereof, and many details have been set forth for purposes of illustration, it will be apparent to those skilled in the art that the invention is susceptible to additional embodiments and that certain of the details described herein may be varied considerably without departing from the basic principles of the invention.

The use of the terms “a” and “an” and “the” and similar referents in the context of describing the invention are to be construed to cover both the singular and the plural, unless otherwise indicated herein or clearly contradicted by context. The terms “comprising,” “having,” “including,” and “containing” are to be construed as open-ended terms (i.e., meaning “including, but not limited to”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Embodiments of this invention are described herein, including the best mode known to the inventors for carrying out the invention. Variations of those embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A method comprising of inhibiting translation of SIN3A in a CF cell, increasing CFTR mRNA expression in a cell, generating a CFTR anion channel in a cell, enhancing anion transport in an epithelial cell, and/or enhancing CFTR protein processing in a cell, comprising contacting the cell with a therapeutic agent, wherein the agent comprises miR-138, a miR-138 mimic, an anti-SIN3A RNAi molecule, and/or an anti-SIN3A antisense oligonucleotide (ASO) or other agent that suppresses SIN3A expression, a small molecule drug that interferes with SIN3A activity or whose actions mimic the biological effects of SIN3A suppression.

2. The method of claim 1, wherein the method comprises inhibiting translation of SIN3A in the CF cell by at least about 10%.

3. (canceled)

4. (canceled)

5. (canceled)

6. (canceled)

7. A method of increased surface display of ΔF508-CFTR protein on a cell by knocking down a gene product level in the cell comprising contacting the cell with a therapeutic agent, wherein the agent comprises miR-138, a miR-138 mimic, an anti-SIN3A RNAi molecule, and/or an anti-SIN3A antisense oligonucleotide (ASO) or other agent that suppresses SIN3A expression, a small molecule drug that interferes with SIN3A activity or whose actions mimic the biological effects of SIN3A suppression, wherein the gene product is produced by a gene listed in Table 6:
TABLE 6

| Ref. No. | Gene ID |
|---|---|
| 1 | DERL1 |
| 2 | HSPA8 |
| 3 | HSPA5 |
| 4 | DNAJB12 |
| 5 | BAG1 |
| 6 | NHERF1 (SLC9A3R1) |
| 7 | CAPNS1 |
| 8 | HSPB1 |
| 9 | HSPA1A |
| 10 | MARCH2 |
| 11 | HAP90B1 |
| 12 | RNF128 |
| 13 | CANX |
| 14 | GRIP1 |
| 15 | SYVN1 |
| 16 | DAB2 |
| 17 | RCN2 |
| 18 | GOPC |
| 19 | HSPA9 |
| 20 | MARCH3 |
| 21 | PPP2R1B |
| 22 | RCN1 |
| 23 | BAG2 |
| 24 | ATP6V1A |
| 25 | DNAJC3 |

8. The method of claim 7, wherein the gene product level in the cell is decreased by 10%.

9. The method of claim 1, wherein the cell is a CF epithelial cell.

10. The method of claim 9 wherein the CF epithelial cell is an airway epithelial cell.

11. The method of claim 10, wherein the airway epithelial cell is a lung cell, a nasal cell, a tracheal cell, a bronchial cell, a bronchiolar or alveolar epithelial cell.

12. The method of claim 10, wherein the airway epithelial cells are present in a mammal.

13. The method of claim 12, wherein the agent is administered orally.

14. The method of claim 12, wherein the agent is administered by inhalation.

15. The method of claim 9, wherein the epithelial cells are intestinal, pancreatic epithelia, liver, gallbladder, reproductive tract, or sweat gland cells.

16. The method of claim 15, wherein the intestinal epithelial cells are present in a mammal.

17. The method according to claim 16, wherein the therapeutic agent is administered orally.

18. The method of claim 1, wherein the therapeutic agent is present within a pharmaceutical composition.

19. A method of claim 1, wherein the cell produces a CFTR protein having a deletion at position 508.

20. A method of treating a subject having cystic fibrosis (CF) comprising administering to the subject an effective amount of a therapeutic agent to alleviate the symptoms of CF, wherein the agent comprises miR-138, a miR-138 mimic, an anti-SIN3A RNAi molecule, and/or an anti-SIN3A antisense oligonucleotide (ASO) or other agent that suppresses SIN3A expression, a small molecule drug that interferes with SIN3A activity or whose actions mimic the biological effects of SIN3A suppression.

21. The method of claim 20, wherein the method increases chloride ion conductance in airway epithelial cells of the subject, and wherein the subject's CFTR protein has a deletion at position 508.

22. The method of claim 20, wherein the subject is a mammal.

23. (canceled)

24. The method of claim 20, wherein the administration is via aerosol, dry powder, bronchoscopic instillation, intra-airway (tracheal or bronchial) aerosol or orally.

25. The method of claim 20, wherein the therapeutic agent is present within a pharmaceutical composition.

26. The method of claim 20, wherein the therapeutic agent is Aminoglutethimide, Biperiden, Diphenhydramine, Rottlerin, Midodrine, Thioridazine, Sulfadimethoxine, neostigmine bromide, Pyridostigmine, pizotifen, tyrophostin (AG-1478), valproic acid, Scriptaid or neomycin.

27. The method of claim 1, wherein the therapeutic agent is not genistein.

28. A pharmaceutical composition for treatment of cystic fibrosis, comprising miR-138, a miR-138 mimic, an anti-SIN3A RNAi molecule, and/or an anti-SIN3A antisense oligonucleotide (ASO) or other agent that suppresses SIN3A expression, a small molecule drug that interferes with SIN3A activity or whose actions mimic the biological effects of SIN3A suppression in combination with a pharmaceutically acceptable carrier, where the composition does not comprise genistein as an active ingredient, and wherein the composition further comprises a CF therapeutic agent.

29. The pharmaceutical composition of claim 28, wherein the therapeutic agent is Aminoglutethimide, Biperiden, Diphenhydramine, Rottlerin, Midodrine, Thioridazine, Sulfadimethoxine, neostigmine bromide, Pyridostigmine, pizotifen, tyrophostin (AG-1478), valproic acid, Scriptaid or neomycin.

30. (canceled)

31. (canceled)


SEQUENCE LISTING (99 sequences)

SEQ ID NO: 1 - RNA, 99 nt, Homo sapiens
cccuggcauggugugguggggcagcugguguugugaaucaggccguugccaaucagagaacggcuacuucacaacaccagggccacaccacacuacagg

SEQ ID NO: 2 - RNA, 84 nt, Homo sapiens
cguugcugcagcugguguugugaaucaggccgacgagcagcgcauccucuuacccggcuauuucacgacaccaggguugcauca

SEQ ID NO: 3 - RNA, 23 nt, Homo sapiens
agcugguguugugaaucaggccg

SEQ ID NO: 4 - RNA, 18 nt, Artificial Sequence
  Description of Artificial Sequence Synthetic oligonucleotide
ugauucacaacaccagcu

SEQ ID NO: 5 - RNA, 23 nt, Artificial Sequence
  Description of Artificial Sequence Synthetic oligonucleotide
agcugguguugugaaucaggccg

SEQ ID NO: 6 - DNA, 25 bp, Artificial Sequence
  Description of Artificial Sequence Synthetic oligonucleotide
gcgauacaugaauucagauacuacc

SEQ ID NO: 7 - RNA, 27 nt, Artificial Sequence
  Description of Artificial Sequence Synthetic oligonucleotide
gguaguaucugaauucauguaucgcuc

SEQ ID NO: 8 - DNA, 25 bp, Artificial Sequence
  Description of Artificial Sequence Synthetic oligonucleotide
ggaagaauucuauucucaauccaat

SEQ ID NO: 9 - RNA, 27 nt, Artificial Sequence
  Description of Artificial Sequence Synthetic oligonucleotide
auuggauugagaauagaauucuuccuu

SEQ ID NO: 10 - DNA, 25 bp, Artificial Sequence
  Description of Artificial Sequence Synthetic oligonucleotide
cguuaaucgcguauaauacgcguat

SEQ ID NO: 11 - RNA, 27 nt, Artificial Sequence
  Description of Artificial Sequence Synthetic oligonucleotide
auacgcguauuauacgcgauuaacgac

SEQ ID NO: 12 - DNA, 23 bp, Artificial Sequence
  Description of Artificial Sequence Synthetic oligonucleotide
cggcctgatucacaacaccagcu

SEQ ID NO: 13 - DNA, 22 bp, Artificial Sequence
  Description of Artificial Sequence Synthetic oligonucleotide
gcguatuatagccgauuaacga

SEQ ID NO: 14 - DNA, 22 bp, Artificial Sequence
  Description of Artificial Sequence Synthetic primer
caacatctagtgagcagtcagg

SEQ ID NO: 15 - DNA, 22 bp, Artificial Sequence
  Description of Artificial Sequence Synthetic primer
cccaggtaagggatgtattgtg

SEQ ID NO: 16 - DNA, 26 bp, Artificial Sequence
  Description of Artificial Sequence Synthetic probe
tccagatcctggaaatcagggttagt

SEQ ID NO: 17 - DNA, 23 bp, Artificial Sequence
  Description of Artificial Sequence Synthetic primer
gcacagaaaccagtatttctccc

SEQ ID NO: 18 - DNA, 22 bp, Artificial Sequence
  Description of Artificial Sequence Synthetic primer
ggtcttcttgctgtttccttcc

SEQ ID NO: 19 - DNA, 25 bp, Artificial Sequence
  Description of Artificial Sequence Synthetic probe
tgctctcgaccacgttgacacttcc

SEQ ID NO: 20 - DNA, 17 bp, Artificial Sequence
  Description of Artificial Sequence Synthetic primer
ggcatggccttccgtgt

SEQ ID NO: 21 - DNA, 18 bp, Artificial Sequence
  Description of Artificial Sequence Synthetic primer
gcccaggatgcccttgag

SEQ ID NO: 22 - DNA, 30 bp, Artificial Sequence
  Description of Artificial Sequence Synthetic probe
cctgcttcaccaccttcttgatgtcatcat

SEQ ID NO: 23 - DNA, 21 bp, Artificial Sequence
  Description of Artificial Sequence Synthetic primer
gactttgctttccttggtcag

SEQ ID NO: 24 - DNA, 24 bp, Artificial Sequence
  Description of Artificial Sequence Synthetic primer
ggcttatatccaacacttcgtggg

SEQ ID NO: 25 - DNA, 26 bp, Artificial Sequence
  Description of Artificial Sequence Synthetic probe
atggtcaaggtcgcaagcttgctggt

SEQ ID NO: 26 - DNA, 18 bp, Artificial Sequence
  Description of Artificial Sequence Synthetic primer
tgtgcagaaggatggagt

SEQ ID NO: 27 - DNA, 20 bp, Artificial Sequence
  Description of Artificial Sequence Synthetic primer
ctggtgcttctctcaggata

SEQ ID NO: 28 - DNA, 25 bp, Artificial Sequence
  Description of Artificial Sequence Synthetic probe
tggaatatgccctgcgtaaactgga

SEQ ID NO: 29 - DNA, 22 bp, Artificial Sequence
  Description of Artificial Sequence Synthetic primer
agtggaggaaagcctttggagt

SEQ ID NO: 30 - DNA, 21 bp, Artificial Sequence
  Description of Artificial Sequence Synthetic primer
acagatctgagcccaacctca

SEQ ID NO: 31 - DNA, 20 bp, Artificial Sequence
  Description of Artificial Sequence Synthetic primer
cccatatgatgtgcctgatt

SEQ ID NO: 32 - DNA, 20 bp, Artificial Sequence
  Description of Artificial Sequence Synthetic primer
gtcggctactcccacgtaaa

SEQ ID NO: 33 - DNA, 25 bp, Artificial Sequence
  Description of Artificial Sequence Synthetic primer
aagtttaaacctgcaaagccagagc

SEQ ID NO: 34 - DNA, 27 bp, Artificial Sequence
  Description of Artificial Sequence Synthetic primer
ttgcggccgcttaagtaagaaccaagc

SEQ ID NO: 35 - DNA, 20 bp, Artificial Sequence
  Description of Artificial Sequence Synthetic primer
gagctaagactggagtctcc

SEQ ID NO: 36 - DNA, 21 bp, Artificial Sequence
  Description of Artificial Sequence Synthetic primer
tgtgcaagcaaactgcatgtc

SEQ ID NO: 37 - DNA, 60 bp, Artificial Sequence
  Description of Artificial Sequence Synthetic primer
gtttgcttgcacacgttaatcgagctaagactggagtctcctgtggcctaactttcaatg

SEQ ID NO: 38 - DNA, 60 bp, Artificial Sequence
  Description of Artificial Sequence Synthetic primer
cattgaaagttaggccacaggagactccagtcttagctcgattaacgtgtgcaagcaaac

SEQ ID NO: 39 - DNA, 22 bp, Artificial Sequence
  Description of Artificial Sequence Synthetic primer
tttactctctgacacacacacg

SEQ ID NO: 40 - DNA, 18 bp, Artificial Sequence
  Description of Artificial Sequence Synthetic primer
gatggcactaaggtagac

SEQ ID NO: 41 - DNA, 47 bp, Artificial Sequence
  Description of Artificial Sequence Synthetic primer
gtctaccttagtgccatccgttaattttactctctgacacacacacg

SEQ ID NO: 42 - DNA, 47 bp, Artificial Sequence
  Description of Artificial Sequence Synthetic primer
cgtgtgtgtgtcagagagtaaaattaacggatggcactaaggtagac

SEQ ID NO: 43 - DNA, 29 bp, Artificial Sequence
  Description of Artificial Sequence Synthetic primer
ggatagtgctgctattactaaaggtttct

SEQ ID NO: 44 - DNA, 20 bp, Artificial Sequence
  Description of Artificial Sequence Synthetic primer
atggcagctccaacacatga

SEQ ID NO: 45 - DNA, 31 bp, Artificial Sequence
  Description of Artificial Sequence Synthetic probe
tctgaagacaacaagccaaagggacaaattt

SEQ ID NO: 46 - DNA, 21 bp, Artificial Sequence
  Description of Artificial Sequence Synthetic primer
ccgggatgttgtttgaagctt

SEQ ID NO: 47 - DNA, 32 bp, Artificial Sequence
  Description of Artificial Sequence Synthetic primer
tttaaatagttgaatagaggacgagatacttt

SEQ ID NO: 48 - DNA, 38 bp, Artificial Sequence
  Description of Artificial Sequence Synthetic probe
atagtattttcttctctcttccttacctgccctctgct

SEQ ID NO: 49 - DNA, 27 bp, Artificial Sequence
  Description of Artificial Sequence Synthetic primer
atccattttcttcaagtctctctccat

SEQ ID NO: 50 - DNA, 24 bp, Artificial Sequence
  Description of Artificial Sequence Synthetic primer
ggaatgaggattgtttatgatttg

SEQ ID NO: 51 - DNA, 36 bp, Artificial Sequence
  Description of Artificial Sequence Synthetic probe
cctctttatggaatctccttttgatttgaactttga

SEQ ID NO: 52 - DNA, 24 bp, Artificial Sequence
  Description of Artificial Sequence Synthetic primer
tcttctttcccattcacctttgtc

SEQ ID NO: 53 - DNA, 25 bp, Artificial Sequence
  Description of Artificial Sequence Synthetic primer
ttttggtttcatttatacgcacatc

SEQ ID NO: 54 - DNA, 36 bp, Artificial Sequence
  Description of Artificial Sequence Synthetic probe
ccattgctgataaagattgctccttctattattcca

SEQ ID NO: 55 - RNA, 97 nt, Bos taurus
cuggcacggugcgguggggcagcugguguugugaaucaggccgucgccaaucagagaacggcuacuucacaacaccagggucacaccccaccccagg

SEQ ID NO: 56 - RNA, 83 nt, Bos taurus
guugcugcagcugguguugugaaucaggccgacgagcagcgcauccucuuacccggcuauuucacgacaccaggguugcauca

SEQ ID NO: 57 - RNA, 62 nt, Canis familiaris
agcugguguugugaaucaggccguugccaaucagagaacggcuacuucacaacaccaggguc

SEQ ID NO: 58 - RNA, 69 nt, Canis familiaris
agcugguguugugaaucaugccgacgagcagcgcauccucuuacccggcuauuucacgacaccaggguu

SEQ ID NO: 59 - RNA, 87 nt, Danio rerio
ugugugcugcagcugguguugugaaucaggccgaugucacacgucagcgauaacccggcuauuucacaacaccaggguggcaccaca

SEQ ID NO: 60 - RNA, 84 nt, Equus caballus
cguugcugcagcugguguugugaaucaggccgacgagcagugcauccucuuacccggcuauuucacgacaccaggguugcauca

SEQ ID NO: 61 - RNA, 59 nt, Equus caballus
agcugguguugugaaucaggccguugccaaucagagaacggcuacuucacaacaccagg

SEQ ID NO: 62 - RNA, 79 nt, Fugu rubripes
gcugcagcugguguugugaaucaggccgaugacagacaccuccuauaagccggcuauuucacaacaccaggguggcacc

SEQ ID NO: 63 - RNA, 96 nt, Gallus gallus
cccugccgggugccgugcagcagcugguguugugaaucaggccgucaccagucggagaacggcuacuucacaacaccaggguggcacugcaccaca

SEQ ID NO: 64 - RNA, 83 nt, Gallus gallus
guugcugcagcugguguugugaaucaggccgacggcaagcgcuuccuacuauccggcuauuucacuacaccaggguugcauca

SEQ ID NO: 65 - RNA, 84 nt, Monodelphis domestica; identical to SEQ ID NO: 2

SEQ ID NO: 66 - RNA, 83 nt, Macaca mulatta
guugcugcagcugguguugugaaucaggccgacaagcagcucauccuauuacccggcuauuucacuacaccaggguugcauca

SEQ ID NO: 67 - RNA, 84 nt, Mus musculus; identical to SEQ ID NO: 2

SEQ ID NO: 68 - RNA, 99 nt, Mus musculus
cucuagcaugguguugugggacagcugguguugugaaucaggccguugccaaucagagaacggcuacuucacaacaccagggccacacugcacugcaag

SEQ ID NO: 69 - RNA, 137 nt, Ornithorhynchus anatinus
gacagagcuuuuaagagaggcacagacgaccugaggcacgauacaaagaagcguggcucuuuccgcccugacuaccgguauggugaagcagcugguguugugaaucaggccgucgccaaucugagaacggcuacuuc

SEQ ID NO: 70 - RNA, 115 nt, Ornithorhynchus anatinus
gacgcucacucugguaucggugcugcagcugguguugugaaucaggccgacgagcagcgaguccuaauacccggcuauuucacuacaccaggguugcaucauaccacuccgcuuc

SEQ ID NO: 71 - RNA, 80 nt, Petromyzon marinus
ccggcggcagcugguguugugaaucaggccgguggcgcaaccccuaaacacacggcuguuucacuacagcauggucgcau

SEQ ID NO: 72 - RNA, 85 nt, Petromyzon marinus
uggugccgugccgcagcugguguugugaaucaggcugauccucuccugcuccuccgccgcuucacagcaccggcacggcacggcc

SEQ ID NO: 73 - RNA, 84 nt, Pongo pygmaeus; identical to SEQ ID NO: 2

SEQ ID NO: 74 - RNA, 83 nt, Pan troglodytes; identical to SEQ ID NO: 56

SEQ ID NO: 75 - RNA, 99 nt, Rattus norvegicus
cucuggcaugguguugugggacagcugguguugugaaucaggccguugccaaucagagaacggcuacuucacaacaccagggucucacugcacugcagg

SEQ ID NO: 76 - RNA, 82 nt, Rattus norvegicus
guugcugcagcugguguugugaaucaggccgacgagcaacgcauccucuuacccggcuauuucacgacaccaggguugcacc

SEQ ID NO: 77 - RNA, 73 nt, Taeniopygia guttata
gcugugcaacagcugguguugugaaucaggccgucaccagucggagaacggcuacuucacaacaccagggucg

SEQ ID NO: 78 - RNA, 93 nt, Taeniopygia guttata
uuguugcugcagcugguguugugaaucaggccgacgacaagcgcuuccuacaauccggcuauuucacuacaccaggguugcaucauaccacuc

SEQ ID NO: 79 - RNA, 79 nt, Tetraodon nigroviridis
gcugcagcugguguugugaaucaggccgaugacagacaccuccuagaagccggcuauuucacaacaccaggguggcacc

SEQ ID NO: 80 - RNA, 85 nt, Xenopus tropicalis
cggugcggagcagcagcugguguugugaaucaggccgugaccacucagaaaacggcuacuucacaacaccaggguugcuucucac

SEQ ID NO: 81 - RNA, 23 nt, Bos taurus
agcugguguugugaaucaggccg

SEQ ID NO: 82 - RNA, 23 nt, Canis familiaris
agcugguguugugaaucaggccg

SEQ ID NO: 83 - RNA, 24 nt, Canis familiaris
agcugguguugugaaucaugccga

SEQ ID NO: 84 - RNA, 22 nt, Danio rerio
agcugguguugugaaucaggcc

SEQ ID NO: 85 - RNA, 23 nt, Equus caballus
agcugguguugugaaucaggccg

SEQ ID NO: 86 - RNA, 22 nt, Fugu rubripes
agcugguguugugaaucaggcc

SEQ ID NO: 87 - RNA, 17 nt, Gallus gallus
agcugguguugugaauc

SEQ ID NO: 88 - RNA, 23 nt, Monodelphis domestica
agcugguguugugaaucaggccg

SEQ ID NO: 89 - RNA, 17 nt, Macaca mulatta
agcugguguugugaauc

SEQ ID NO: 90 - RNA, 23 nt, Mus musculus
agcugguguugugaaucaggccg

SEQ ID NO: 91 - RNA, 23 nt, Ornithorhynchus anatinus
agcugguguugugaaucaggccg

SEQ ID NO: 92 - RNA, 23 nt, Petromyzon marinus
agcugguguugugaaucaggccg

SEQ ID NO: 93 - RNA, 23 nt, Petromyzon marinus
agcugguguugugaaucaggcug

SEQ ID NO: 94 - RNA, 23 nt, Pongo pygmaeus
agcugguguugugaaucaggccg

SEQ ID NO: 95 - RNA, 23 nt, Pan troglodytes
agcugguguugugaaucaggccg

SEQ ID NO: 96 - RNA, 23 nt, Rattus norvegicus
agcugguguugugaaucaggccg

SEQ ID NO: 97 - RNA, 23 nt, Taeniopygia guttata
agcugguguugugaaucaggccg

SEQ ID NO: 98 - RNA, 22 nt, Tetraodon nigroviridis
agcugguguugugaaucaggcc

SEQ ID NO: 99 - RNA, 17 nt, Xenopus tropicalis
agcugguguugugaauc